SEQUENCE LISTING (179 sequences)

SEQ ID NO: 1 - DNA, 3795 bp, synthetic construct
  source 1..3795: mol_type = other DNA; note = Rep2-AAP
ggtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttaacttccagattgactcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggatagaagaggcctgtagagtagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataactaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 2 - protein, 736 aa, wild-type adeno-associated virus serotype 9 (AAV9)
  source 1..736: note = wild-type AAV9 VP1 amino acid sequence (underlined: position 586-588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 743 aa, synthetic construct
  source 1..743: note = published MyoAAV 1A VP1 amino acid sequence (underlined : corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLTTPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 743 aa, synthetic construct
  source 1..743: note = published MyoAAV 2A VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGPGRGDQTTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 743 aa, synthetic construct
  source 1..743: note = published MyoAAV 4A VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 743 aa, synthetic construct
  source 1..743: note = AVT901 VP1 amino acid sequence (underlined: corresponding to position 586-588 of wild-type AAV9 VP1 amino acid sequence; italic: insert after wild-type position 453)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGHMNGRNNSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 743 aa, synthetic construct
  source 1..743: note = AVT902 VP1 amino acid sequence (underlined: corresponding to position 586-588 of wild-type AAV9 VP1 amino acid sequence; italic: insert after wild-type position 453)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGPHYTHGISGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 745 aa, synthetic construct
  source 1..745: note = AVT903 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDTNNTNMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 743 aa, synthetic construct
  source 1..743: note = AVT905 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSRGDRPMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 743 aa, synthetic construct
  source 1..743: note = AVT906 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLHKGIDIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 750 aa, synthetic construct
  source 1..750: note = AVT907 VP1 amino acid sequence (underlined: corresponding to position 586-588 of wild-type AAV9 VP1 amino acid sequence; italic: insert after wild-type position 453; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGHMNGRNNSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGPGRGDQTTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 743 aa, synthetic construct; identical to SEQ ID NO: 9
  source 1..743: note = AVT908 VP1 amino acid sequence (underlined: corresponding to position 586-588 of wild-type AAV9 VP1 amino acid sequence; italic and underlined: insert after wild-type position 588)

SEQ ID NO: 13 - protein, 743 aa, synthetic construct
  source 1..743: note = AVT909 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGVNGRSTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 743 aa, synthetic construct
  source 1..743: note = AVT910 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGVNGRQVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 743 aa, synthetic construct
  source 1..743: note = AVT911 VP1 amino acid sequence (underlined: corresponding to position 586-588 of wild-type AAV9 VP1 amino acid sequence; italic: insert after wild-type position 453)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGQTYTHNKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 750 aa, synthetic construct
  source 1..750: note = AVT912 VP1 amino acid sequence (underlined: corresponding to position 586-588 of wild-type AAV9 VP1 amino acid sequence; italic: insert after wild-type position 453; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGQTYTHNKSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSRGDRPMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 743 aa, synthetic construct
  source 1..743: note = AVT913 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 743 aa, synthetic construct
  source 1..743: note = AVT914 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 743 aa, synthetic construct
  source 1..743: note = AVT915 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 743 aa, synthetic construct
  source 1..743: note = AVT916 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 743 aa, synthetic construct
  source 1..743: note = AVT917 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNARGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 743 aa, synthetic construct
  source 1..743: note = AVT918 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, synthetic construct
  source 1..743: note = AVT919 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, synthetic construct
  source 1..743: note = AVT920 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTNMRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 743 aa, synthetic construct
  source 1..743: note = AV9ML001 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDNNYLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 743 aa, synthetic construct
  source 1..743: note = AV9ML002 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 743 aa, synthetic construct
  source 1..743: note = AV9ML004 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNERGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 743 aa, synthetic construct
  source 1..743: note = AV9ML005 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 743 aa, synthetic construct
  source 1..743: note = AV9ML007 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENKRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 743 aa, synthetic construct
  source 1..743: note = AV9ML012 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQINSRGDHNVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 743 aa, synthetic construct
  source 1..743: note = AV9ML014 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 743 aa, synthetic construct
  source 1..743: note = AV9ML016 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDKLSIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 745 aa, synthetic construct
  source 1..745: note = AV9ML018 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTNNSFTKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 7 aa, synthetic construct
  source 1..7: note = published MyoAAV 1A VP1's insert peptide amino acid sequence
RGDLTTP

SEQ ID NO: 35 - protein, 7 aa, synthetic construct
  source 1..7: note = published MyoAAV 2A VP1's insert peptide amino acid sequence
RGDQTTL

SEQ ID NO: 36 - protein, 7 aa, synthetic construct
  source 1..7: note = published MyoAAV 4A VP1's insert peptide amino acid sequence
RGDYNSL

SEQ ID NO: 37 - protein, 7 aa, synthetic construct
  source 1..7: note = AVT901 VP1's insert peptide amino acid sequence
HMNGRNN

SEQ ID NO: 38 - DNA, 21 bp, synthetic construct
  source 1..21: mol_type = other DNA; note = nucleotide sequence encoding AVT901 VP1's insert peptide
catatgaatgggcgtaataat

SEQ ID NO: 39 - protein, 7 aa, synthetic construct
  source 1..7: note = AVT902 VP1's insert peptide amino acid sequence
PHYTHGI

SEQ ID NO: 40 - DNA, 21 bp, synthetic construct
  source 1..21: mol_type = other DNA; note = nucleotide sequence encoding AVT902 VP1's insert peptide
cctcattatacgcatggtatt

SEQ ID NO: 41 - protein, 9 aa, synthetic construct
  source 1..9: note = AVT903 VP1's insert peptide amino acid sequence
RGDTNNTNM

SEQ ID NO: 42 - DNA, 27 bp, synthetic construct
  source 1..27: mol_type = other DNA; note = nucleotide sequence encoding AVT903 VP1's insert peptide
aggggtgatactaataatactaatatg

SEQ ID NO: 43 - protein, 7 aa, synthetic construct
  source 1..7: note = AVT905 VP1's insert peptide amino acid sequence
SRGDRPM

SEQ ID NO: 44 - DNA, 21 bp, synthetic construct
  source 1..21: mol_type = other DNA; note = nucleotide sequence encoding AVT905 VP1's insert peptide
tctaggggtgatcggcctatg

SEQ ID NO: 45 - protein, 7 aa, synthetic construct
  source 1..7: note = AVT906 VP1's insert peptide amino acid sequence
LHKGIDI

SEQ ID NO: 46 - DNA, 21 bp, synthetic construct
  source 1..21: mol_type = other DNA; note = nucleotide sequence encoding AVT906 VP1's insert peptide
ctgcataaggggattgatatt

SEQ ID NO: 47 - protein, 7 aa, synthetic construct
  source 1..7: note = AVT907 VP1's insert peptide (after wild-type position 453) amino acid sequence
HMNGRNN

SEQ ID NO: 48 - DNA, 21 bp, synthetic construct
  source 1..21: mol_type = other DNA; note = nucleotide sequence encoding AVT907 VP1's insert peptide (after wild-type position 453)
catatgaatgggcgtaataat

SEQ ID NO: 49 - protein, 10 aa, synthetic construct
  source 1..10: note = AVT907 VP1's amino acid sequence corresponding to position 586-588 of wild-type AAV9 VP1, plus insert peptide (after wild-type position 588)
GPGRGDQTTL

SEQ ID NO: 50 - DNA, 30 bp, synthetic construct
  source 1..30: mol_type = other DNA; note = nucleotide sequence encoding AVT907 VP1's corresponding to position 586-588 of wild-type AAV9 VP1, and insert after wild-type position 588
ggccccggcagaggagaccagaccaccctg

SEQ ID NO: 51 - protein, 7 aa, synthetic construct
  source 1..7: note = AVT908 VP1's insert peptide amino acid sequence
SRGDRPM

SEQ ID NO: 52 - DNA, 21 bp, synthetic construct
  source 1..21: mol_type = other DNA; note = nucleotide sequence encoding AVT908 VP1's insert peptide
tccagaggcgaccggcctatg

SEQ ID NO: 53 - protein, 7 aa, synthetic construct
  source 1..7: note = AVT909 VP1's insert peptide amino acid sequence
GVNGRST

SEQ ID NO: 54 - DNA, 21 bp, synthetic construct
  source 1..21: mol_type = other DNA; note = nucleotide sequence encoding AVT909 VP1's insert peptide
ggtgtgaatgggaggagtact

SEQ ID NO: 55 - protein, 7 aa, synthetic construct
  source 1..7: note = AVT910 VP1's insert peptide amino acid sequence
GVNGRQV

SEQ ID NO: 56 - DNA, 21 bp, synthetic construct
  source 1..21: mol_type = other DNA; note = nucleotide sequence encoding AVT910 VP1's insert peptide
ggggttaatgggcggcaggtg

SEQ ID NO: 57 - protein, 7 aa, synthetic construct
  source 1..7: note = AVT911 VP1's insert peptide amino acid sequence
QTYTHNK

SEQ ID NO: 58 - DNA, 21 bp, synthetic construct
  source 1..21: mol_type = other DNA; note = nucleotide sequence encoding AVT911 VP1's insert peptide
cagacgtatacgcataataag

SEQ ID NO: 59 - protein, 7 aa, synthetic construct
  source 1..7: note = AVT912 VP1's insert peptide (after wild-type position 453) amino acid sequence
QTYTHNK

SEQ ID NO: 60 - DNA, 21 bp, synthetic construct
  source 1..21: mol_type = other DNA; note = nucleotide sequence encoding AVT912 VP1's insert after wild-type position 453 peptide
cagacgtatacgcataataag

SEQ ID NO: 61 - protein, 7 aa, synthetic construct
  source 1..7: note = AVT912 VP1's insert peptide (after wild-type position 588) amino acid sequence
SRGDRPM

SEQ ID NO: 62 - DNA, 21 bp, synthetic construct
  source 1..21: mol_type = other DNA; note = nucleotide sequence encoding AVT912 VP1's insert peptide (after wild-type position 588)
tctaggggtgatcggcctatg

SEQ ID NO: 63 - protein, 10 aa, synthetic construct
  source 1..10: note = AVT913 VP1's amino acid sequence corresponding to position 586-588 of wild-type AAV9 VP1 plus insert peptide
ENRRGDTNAL

SEQ ID NO: 64 - DNA, 36 bp, synthetic construct
  source 1..36: mol_type = other DNA; note = nucleotide sequence encoding AVT913 VP1's amino acid sequence corresponding to position 586-588 of wild-type AAV9 VP1 plus insert peptide
caggagaacaggagaggagacacgaacgctctggca

SEQ ID NO: 65 - protein, 10 aa, synthetic construct
  source 1..10: note = AVT914 VP1's amino acid sequence corresponding to position 586-588 of wild-type AAV9 VP1 plus insert peptide
NNSRGDHNSL

SEQ ID NO: 66 - DNA, 36 bp, synthetic construct
  source 1..36: mol_type = other DNA; note = nucleotide sequence encoding AVT914 VP1's amino acid sequence corresponding to position 586-588 of wild-type AAV9 VP1 plus insert peptide
cagaataactcgagaggagaccataactcgctggca

SEQ ID NO: 67 - protein, 10 aa, synthetic construct
  source 1..10: note = AVT915 VP1's amino acid sequence corresponding to position 586-588 of wild-type AAV9 VP1 plus insert peptide
NNSRGDYNSL

SEQ ID NO: 68 - DNA, 36 bp, synthetic construct
  source 1..36: mol_type = other DNA; note = nucleotide sequence encoding AVT915 VP1's amino acid sequence corresponding to position 586-588 of wild-type AAV9 VP1 plus insert peptide
cagaataacagtagaggagactataacagtctggca

SEQ ID NO: 69 - protein, 10 aa, synthetic construct
  source 1..10: note = AVT916 VP1's amino acid sequence corresponding to position 586-588 of wild-type AAV9 VP1 plus insert peptide
NNLRGDHNSL

SEQ ID NO: 70 - DNA, 36 bp, synthetic construct
  source 1..36: mol_type = other DNA; note = nucleotide sequence encoding AVT916 VP1's amino acid sequence corresponding to position 586-588 of wild-type AAV9 VP1 plus insert peptide
cagaataacctgagaggagaccataactctctggca

SEQ ID NO: 71 - protein, 10 aa, synthetic construct
  source 1..10: note = AVT917 VP1's amino acid sequence corresponding to position 586-588 of wild-type AAV9 VP1 plus insert peptide
NNARGDHNSL

SEQ ID NO: 72 - DNA, 36 bp, synthetic construct
  source 1..36: mol_type = other DNA; note = nucleotide sequence encoding AVT917 VP1's amino acid sequence corresponding to position 586-588 of wild-type AAV9 VP1 plus insert peptide
cagaataacgctagaggagaccataacagtctggca

SEQ ID NO: 73 - protein, 10 aa, synthetic construct
  source 1..10: note = AVT918 VP1's amino acid sequence corresponding to position 586-588 of wild-type AAV9 VP1 plus insert peptide
GNIRGDHNTL

SEQ ID NO: 74 - DNA, 36 bp, synthetic construct
  source 1..36: mol_type = other DNA; note = nucleotide sequence encoding AVT918 VP1's amino acid sequence corresponding to position 586-588 of wild-type AAV9 VP1 plus insert peptide
cagggtaacattagaggagaccataacactctggca

SEQ ID NO: 75 - protein, 10 aa, synthetic construct
  source 1..10: note = AVT919 VP1's amino acid sequence corresponding to position 586-588 of wild-type AAV9 VP1 plus insert peptide
MNVRGDTNKL

SEQ ID NO: 76 - DNA, 36 bp, synthetic construct
  source 1..36: mol_type = other DNA; note = nucleotide sequence encoding AVT919 VP1's amino acid sequence corresponding to position 586-588 of wild-type AAV9 VP1 plus insert peptide
cagatgaacgtgagaggagacactaacaagctggca

SEQ ID NO: 77 - protein, 10 aa, synthetic construct
  source 1..10: note = AVT920 VP1's amino acid sequence corresponding to position 586-588 of wild-type AAV9 VP1 plus insert peptide
TNMRGDHNAL

SEQ ID NO: 78 - DNA, 36 bp, synthetic construct
  source 1..36: mol_type = other DNA; note = nucleotide sequence encoding AVT920 VP1's amino acid sequence corresponding to position 586-588 of wild-type AAV9 VP1 plus insert peptide
cagacgaacatgagaggagaccataacgcgctggca

SEQ ID NO: 79 - protein, 10 aa, synthetic construct
  source 1..10: note = AV9ML001 VP1's amino acid sequence corresponding to position 586-588 of wild-type AAV9 VP1 plus insert peptide
NNNRGDNNYL

SEQ ID NO: 80 - protein, 10 aa, synthetic construct
  source 1..10: note = AV9ML002 VP1's amino acid sequence corresponding to position 586-588 of wild-type AAV9 VP1 plus insert peptide
NNNRGDHNTL

SEQ ID NO: 81 - protein, 10 aa, synthetic construct
  source 1..10: note = AV9ML004 VP1's amino acid sequence corresponding to position 586-588 of wild-type AAV9 VP1 plus insert peptide
NNERGDRNSL

SEQ ID NO: 82 - protein, 10 aa, synthetic construct
  source 1..10: note = AV9ML005 VP1's amino acid sequence corresponding to position 586-588 of wild-type AAV9 VP1 plus insert peptide
NNSRGDHNTL

SEQ ID NO: 83 - protein, 10 aa, synthetic construct
  source 1..10: note = AV9ML007 VP1's amino acid sequence corresponding to position 586-588 of wild-type AAV9 VP1 plus insert peptide
ENKRGDHNSL

SEQ ID NO: 84 - protein, 10 aa, synthetic construct
  source 1..10: note = AV9ML012 VP1's amino acid sequence corresponding to position 586-588 of wild-type AAV9 VP1 plus insert peptide
INSRGDHNVL

SEQ ID NO: 85 - protein, 10 aa, synthetic construct
  source 1..10: note = AV9ML014 VP1's amino acid sequence corresponding to position 586-588 of wild-type AAV9 VP1 plus insert peptide
NNNRGDYNSL

SEQ ID NO: 86 - protein, 7 aa, synthetic construct
  source 1..7: note = AV9ML016 VP1's insert peptide amino acid sequence
RGDKLSI

SEQ ID NO: 87 - protein, 9 aa, synthetic construct
  source 1..9: note = AV9ML018 VP1's insert peptide amino acid sequence
LNTNNSFTK

SEQ ID NO: 88-90 - skipped (3 entries)

SEQ ID NO: 91 - protein, 12 aa, synthetic construct
  source 1..12: note = Amino acid sequence illustrating the substitution of positions 586-588 of wild-type AAV9 VP1 and insertion of a 7-mer peptide after position 588 to generate Library #4 (X stands for any amino acid residue)
  VARIANT 2: note = X stands for any amino acid residue
  VARIANT 4: note = X stands for any amino acid residue
  VARIANT 8: note = X stands for any amino acid residue
  VARIANT 10: note = X stands for any amino acid residue
QXNXRGDXNXLA

SEQ ID NO: 92 - protein, 26 aa, synthetic construct
  source 1..26: note = Cap9-453-7AA-PF primer nucleotide sequence
TTCACGGACTCAGACTATCAGCTCCC

SEQ ID NO: 93 - DNA, 69 bp, synthetic construct
  source 1..69: mol_type = other DNA; note = Cap9-453-7AA-PR primer nucleotide sequence (N stands for A, G, T or C; M stands for A or C)
ttttagcgtttgttgattctgtccagamnnmnnmnnmnnmnnmnnmnnaccgttaatagtctttgagag

SEQ ID NO: 94 - DNA, 28 bp, synthetic construct
  source 1..28: mol_type = other DNA; note = Cap9-588-7AA-PF primer nucleotide sequence
taatagcttgatgaatcctggacctgcc

SEQ ID NO: 95 - DNA, 72 bp, synthetic construct
  source 1..72: mol_type = other DNA; note = Cap9-588-7AA-PR primer nucleotide sequence (N stands for A, G, T or C; M stands for A or C)
ttttgaacccagccggtctgcgcctgtgcmnnmnnmnnmnnmnnmnnmnnttgggcactctggtggtttgtg

SEQ ID NO: 96 - DNA, 28 bp, synthetic construct
  source 1..28: mol_type = other DNA; note = Cap9-588-9AA-PF primer nucleotide sequence
taatagcttgatgaatcctggacctgcc

SEQ ID NO: 97 - DNA, 78 bp, synthetic construct
  source 1..78: mol_type = other DNA; note = Cap9-588-9AA-PR primer nucleotide sequence (N stands for A, G, T or C; M stands for A or C)
ttttgaacccagccggtctgcgcctgtgcmnnmnnmnnmnnmnnmnnmnnmnnmnnttgggcactctggtggtttgtg

SEQ ID NO: 98 - DNA, 28 bp, synthetic construct
  source 1..28: mol_type = other DNA; note = Cap9-R4A-PF primer nucleotide sequence
taatagcttgatgaatcctggacctgcc

SEQ ID NO: 99 - DNA, 72 bp, synthetic construct
  source 1..72: mol_type = other DNA; note = Cap9-R4A-PR primer nucleotide sequence (N stands for A, G, T or C; M stands for A or C)
ttttgaacccagccggtctgcgcctgtgccagmnngttmnngtctcctctmnngttmnnctggtggtttgtg

SEQ ID NO: 100 - DNA, 30 bp, synthetic construct
  source 1..30: mol_type = other DNA; note = nucleotide sequence encoding AV9ML001 VP1's amino acid sequence corresponding to position 586-588 of wild-type AAV9 VP1 plus insert peptide
aacaacaacagaggcgacaacaactacctc

SEQ ID NO: 101 - DNA, 30 bp, synthetic construct
  source 1..30: mol_type = other DNA; note = nucleotide sequence encoding AV9ML002 VP1's amino acid sequence corresponding to position 586-588 of wild-type AAV9 VP1 plus insert peptide
aacaacaacagaggcgaccacaacaccctc

SEQ ID NO: 102 - DNA, 30 bp, synthetic construct
  source 1..30: mol_type = other DNA; note = nucleotide sequence encoding AV9ML004 VP1's amino acid sequence corresponding to position 586-588 of wild-type AAV9 VP1 plus insert peptide
aacaacgaaagaggcgacagaaacagcctc

SEQ ID NO: 103 - DNA, 30 bp, synthetic construct
  source 1..30: mol_type = other DNA; note = nucleotide sequence encoding AV9ML005 VP1's amino acid sequence corresponding to position 586-588 of wild-type AAV9 VP1 plus insert peptide
aacaacagcagaggcgaccacaacaccctc

SEQ ID NO: 104 - DNA, 30 bp, synthetic construct
  source 1..30: mol_type = other DNA; note = nucleotide sequence encoding AV9ML007 VP1's amino acid sequence corresponding to position 586-588 of wild-type AAV9 VP1 plus insert peptide
gaaaacaaaagaggcgaccacaacagcctc

SEQ ID NO: 105 - DNA, 30 bp, synthetic construct
  source 1..30: mol_type = other DNA; note = nucleotide sequence encoding AV9ML012 VP1's amino acid sequence corresponding to position 586-588 of wild-type AAV9 VP1 plus insert peptide
atcaacagcagaggcgaccacaacgtcctc

SEQ ID NO: 106 - DNA, 30 bp, synthetic construct
  source 1..30: mol_type = other DNA; note = nucleotide sequence encoding AV9ML014 VP1's amino acid sequence corresponding to position 586-588 of wild-type AAV9 VP1 plus insert peptide
aacaacaacagaggcgactacaacagcctc

SEQ ID NO: 107 - DNA, 21 bp, synthetic construct
  source 1..21: mol_type = other DNA; note = nucleotide sequence encoding AV9ML016 VP1's amino acid sequence corresponding to position 586-588 of wild-type AAV9 VP1 plus insert peptide
agaggcgacaaactcagcatc

SEQ ID NO: 108 - DNA, 27 bp, synthetic construct
  source 1..27: mol_type = other DNA; note = nucleotide sequence encoding AV9ML018 VP1's amino acid sequence corresponding to position 586-588 of wild-type AAV9 VP1 plus insert peptide
ctcaacaccaacaacagcttcaccaaa

SEQ ID NO: 109 - protein, 10 aa, synthetic construct
  source 1..10: note = published MyoAAV 1A VP1's amino acid sequence corresponding to position 586-588 of wild-type AAV9 VP1 plus insert peptide
SAQRGDLTTP

SEQ ID NO: 110 - protein, 10 aa, synthetic construct
  source 1..10: note = published MyoAAV 2A VP1's amino acid sequence corresponding to position 586-588 of wild-type AAV9 VP1 plus insert peptide
GPGRGDQTTL

SEQ ID NO: 111 - protein, 743 aa, synthetic construct
  source 1..743: note = published MyoAAV 3A VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYVGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 112 - protein, 7 aa, synthetic construct
  source 1..7: note = published MyoAAV 3A VP1's insert peptide amino acid sequence
RGDYVGL

SEQ ID NO: 113 - protein, 10 aa, synthetic construct
  source 1..10: note = published MyoAAV 3A VP1's amino acid sequence corresponding to position 586-588 of wild-type AAV9 VP1 plus insert peptide
SAQRGDYVGL

SEQ ID NO: 114 - protein, 10 aa, synthetic construct
  source 1..10: note = published MyoAAV 4A VP1's amino acid sequence corresponding to position 586-588 of wild-type AAV9 VP1 plus insert peptide
SNSRGDYNSL

SEQ ID NO: 115 - protein, 743 aa, synthetic construct
  source 1..743: note = AV9ML025 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - protein, 10 aa, synthetic construct
  source 1..10: note = AV9ML025 VP1's amino acid sequence corresponding to position 586-588 of wild-type AAV9 VP1 plus insert peptide insert peptide
SNSRGDHNTL

SEQ ID NO: 117 - protein, 743 aa, synthetic construct
  source 1..743: note = AV9ML026 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 118 - protein, 10 aa, synthetic construct
  source 1..10: note = AV9ML026 VP1's amino acid sequence corresponding to position 586-588 of wild-type AAV9 VP1 plus insert peptide insert peptide
SNSRGDHNSL

SEQ ID NO: 119 - protein, 743 aa, synthetic construct
  source 1..743: note = AV9ML027 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - protein, 10 aa, synthetic construct
  source 1..10: note = AV9ML027 VP1's amino acid sequence corresponding to position 586-588 of wild-type AAV9 VP1 plus insert peptide insert peptide
ENRRGDHNSL

SEQ ID NO: 121 - protein, 743 aa, synthetic construct
  source 1..743: note = AV9ML028 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 122 - protein, 10 aa, synthetic construct
  source 1..10: note = AV9ML028 VP1's amino acid sequence corresponding to position 586-588 of wild-type AAV9 VP1 plus insert peptide insert peptide
GNSRGDYNSL

SEQ ID NO: 123 - protein, 743 aa, synthetic construct
  source 1..743: note = AV9ML029 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNSRGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 10 aa, synthetic construct
  source 1..10: note = AV9ML029 VP1's amino acid sequence corresponding to position 586-588 of wild-type AAV9 VP1 plus insert peptide insert peptide
NNSRGDRNSL

SEQ ID NO: 125 - protein, 743 aa, synthetic construct
  source 1..743: note = AV9ML030 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 126 - protein, 10 aa, synthetic construct
  source 1..10: note = AV9ML030 VP1's amino acid sequence corresponding to position 586-588 of wild-type AAV9 VP1 plus insert peptide insert peptide
MNVRGDTNSL

SEQ ID NO: 127 - protein, 743 aa, synthetic construct
  source 1..743: note = AV9ML031 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNARGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 128 - protein, 10 aa, synthetic construct
  source 1..10: note = AV9ML031 VP1's amino acid sequence corresponding to position 586-588 of wild-type AAV9 VP1 plus insert peptide insert peptide
SNARGDHNSL

SEQ ID NO: 129 - protein, 743 aa, synthetic construct
  source 1..743: note = AV9ML032 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 130 - protein, 10 aa, synthetic construct
  source 1..10: note = AV9ML032 VP1's amino acid sequence corresponding to position 586-588 of wild-type AAV9 VP1 plus insert peptide insert peptide
GNSRGDHNTL

SEQ ID NO: 131 - protein, 743 aa, synthetic construct
  source 1..743: note = AV9ML033 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNNRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 132 - protein, 10 aa, synthetic construct
  source 1..10: note = AV9ML033 VP1's amino acid sequence corresponding to position 586-588 of wild-type AAV9 VP1 plus insert peptide insert peptide
NNNRGDHNAL

SEQ ID NO: 133 - protein, 743 aa, synthetic construct
  source 1..743: note = AV9ML034 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 134 - protein, 10 aa, synthetic construct
  source 1..10: note = AV9ML034 VP1's amino acid sequence corresponding to position 586-588 of wild-type AAV9 VP1 plus insert peptide insert peptide
NNLRGDRNSL

SEQ ID NO: 135 - protein, 743 aa, synthetic construct
  source 1..743: note = AV9ML035 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDNRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 136 - protein, 10 aa, synthetic construct
  source 1..10: note = AV9ML035 VP1's amino acid sequence corresponding to position 586-588 of wild-type AAV9 VP1 plus insert peptide insert peptide
DNRRGDTNAL

SEQ ID NO: 137 - protein, 743 aa, synthetic construct
  source 1..743: note = AV9ML036 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENERGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 138 - protein, 10 aa, synthetic construct
  source 1..10: note = AV9ML036 VP1's amino acid sequence corresponding to position 586-588 of wild-type AAV9 VP1 plus insert peptide insert peptide
ENERGDHNSL

SEQ ID NO: 139 - protein, 743 aa, synthetic construct
  source 1..743: note = AV9ML037 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNARGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 140 - protein, 10 aa, synthetic construct
  source 1..10: note = AV9ML037 VP1's amino acid sequence corresponding to position 586-588 of wild-type AAV9 VP1 plus insert peptide insert peptide
NNARGDTNAL

SEQ ID NO: 141 - protein, 743 aa, synthetic construct
  source 1..743: note = AV9ML038 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNLRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 142 - protein, 10 aa, synthetic construct
  source 1..10: note = AV9ML038 VP1's amino acid sequence corresponding to position 586-588 of wild-type AAV9 VP1 plus insert peptide insert peptide
SNLRGDHNSL

SEQ ID NO: 143 - protein, 743 aa, synthetic construct
  source 1..743: note = AV9ML039 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMSVRGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 144 - protein, 10 aa, synthetic construct
  source 1..10: note = AV9ML039 VP1's amino acid sequence corresponding to position 586-588 of wild-type AAV9 VP1 plus insert peptide insert peptide
MSVRGDTNKL

SEQ ID NO: 145 - protein, 743 aa, synthetic construct
  source 1..743: note = AV9ML040 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNVRGDTNKPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 146 - protein, 10 aa, synthetic construct
  source 1..10: note = AV9ML040 VP1's amino acid sequence corresponding to position 586-588 of wild-type AAV9 VP1 plus insert peptide insert peptide
MNVRGDTNKP

SEQ ID NO: 147 - protein, 743 aa, synthetic construct
  source 1..743: note = AV9ML041 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNLRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 148 - protein, 10 aa, synthetic construct
  source 1..10: note = AV9ML041 VP1's amino acid sequence corresponding to position 586-588 of wild-type AAV9 VP1 plus insert peptide insert peptide
NNLRGDTNAL

SEQ ID NO: 149 - protein, 743 aa, synthetic construct
  source 1..743: note = AV9ML042 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQINSRGDYNVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 150 - protein, 10 aa, synthetic construct
  source 1..10: note = AV9ML042 VP1's amino acid sequence corresponding to position 586-588 of wild-type AAV9 VP1 plus insert peptide insert peptide
INSRGDYNVL

SEQ ID NO: 151 - protein, 743 aa, synthetic construct
  source 1..743: note = AV9ML043 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQKNRRGDTNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 10 aa, synthetic construct
  source 1..10: note = AV9ML043 VP1's amino acid sequence corresponding to position 586-588 of wild-type AAV9 VP1 plus insert peptide insert peptide
KNRRGDTNAL

SEQ ID NO: 153 - protein, 743 aa, synthetic construct
  source 1..743: note = AV9ML044 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENERGDRNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 154 - protein, 10 aa, synthetic construct
  source 1..10: note = AV9ML044 VP1's amino acid sequence corresponding to position 586-588 of wild-type AAV9 VP1 plus insert peptide insert peptide
ENERGDRNSL

SEQ ID NO: 155 - protein, 743 aa, synthetic construct
  source 1..743: note = AV9ML045 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMNARGDTNKLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 156 - protein, 10 aa, synthetic construct
  source 1..10: note = AV9ML045 VP1's amino acid sequence corresponding to position 586-588 of wild-type AAV9 VP1 plus insert peptide insert peptide
MNARGDTNKL

SEQ ID NO: 157 - protein, 743 aa, synthetic construct
  source 1..743: note = AV9ML046 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDRNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 158 - protein, 10 aa, synthetic construct
  source 1..10: note = AV9ML046 VP1's amino acid sequence corresponding to position 586-588 of wild-type AAV9 VP1 plus insert peptide insert peptide
GNIRGDRNTL

SEQ ID NO: 159 - protein, 743 aa, synthetic construct
  source 1..743: note = AV9ML047 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGNIRGDHNALAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 160 - protein, 10 aa, synthetic construct
  source 1..10: note = AV9ML047 VP1's amino acid sequence corresponding to position 586-588 of wild-type AAV9 VP1 plus insert peptide insert peptide
GNIRGDHNAL

SEQ ID NO: 161 - protein, 743 aa, synthetic construct
  source 1..743: note = AV9ML048 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNNRRGDHNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 10 aa, synthetic construct
  source 1..10: note = AV9ML048 VP1's amino acid sequence corresponding to position 586-588 of wild-type AAV9 VP1 plus insert peptide insert peptide
NNRRGDHNSL

SEQ ID NO: 163 - protein, 743 aa, synthetic construct
  source 1..743: note = AV9ML049 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTNIRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 164 - protein, 10 aa, synthetic construct
  source 1..10: note = AV9ML049 VP1's amino acid sequence corresponding to position 586-588 of wild-type AAV9 VP1 plus insert peptide insert peptide
TNIRGDYNSL

SEQ ID NO: 165 - protein, 743 aa, synthetic construct
  source 1..743: note = AV9ML050 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQRNLRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 10 aa, synthetic construct
  source 1..10: note = AV9ML050 VP1's amino acid sequence corresponding to position 586-588 of wild-type AAV9 VP1 plus insert peptide insert peptide
RNLRGDHNTL

SEQ ID NO: 167 - protein, 743 aa, synthetic construct
  source 1..743: note = AV9ML051 VP1 amino acid sequence (underlined: corresponding to position 586-588 of wild-type AAV9 VP1 amino acid sequence; italic and underlined: insert after wild-type position 588)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQCNSRGDHNTLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 168 - protein, 10 aa, synthetic construct
  source 1..10: note = AV9ML051 VP1's amino acid sequence corresponding to position 586-588 of wild-type AAV9 VP1 plus insert peptide insert peptide
CNSRGDHNTL

SEQ ID NO: 169 - protein, 12 aa, synthetic construct
  source 1..12: note = AVT903 VP1's amino acid sequence corresponding to position 586-588 of wild-type AAV9 VP1 plus insert peptide
SAQRGDTNNTNM

SEQ ID NO: 170 - protein, 10 aa, synthetic construct
  source 1..10: note = AVT905 VP1's amino acid sequence corresponding to position 586-588 of wild-type AAV9 VP1 plus insert peptide
SAQSRGDRPM

SEQ ID NO: 171 - protein, 10 aa, synthetic construct
  source 1..10: note = AVT906 VP1's amino acid sequence corresponding to position 586-588 of wild-type AAV9 VP1 plus insert peptide
SAQLHKGIDI

SEQ ID NO: 172 - protein, 10 aa, synthetic construct
  source 1..10: note = AVT908 VP1's amino acid sequence corresponding to position 586-588 of wild-type AAV9 VP1 plus insert peptide
SAQSRGDRPM

SEQ ID NO: 173 - protein, 10 aa, synthetic construct
  source 1..10: note = AVT909 VP1's amino acid sequence corresponding to position 586-588 of wild-type AAV9 VP1 plus insert peptide
SAQGVNGRST

SEQ ID NO: 174 - protein, 10 aa, synthetic construct
  source 1..10: note = AVT910 VP1's amino acid sequence corresponding to position 586-588 of wild-type AAV9 VP1 plus insert peptide
SAQGVNGRQV

SEQ ID NO: 175 - protein, 10 aa, synthetic construct
  source 1..10: note = AVT912 VP1's amino acid sequence corresponding to position 586-588 of wild-type AAV9 VP1 plus insert peptide afterwards
SAQSRGDRPM

SEQ ID NO: 176 - protein, 10 aa, synthetic construct
  source 1..10: note = AV9ML016 VP1's amino acid sequence corresponding to position 586-588 of wild-type AAV9 VP1 plus insert peptide
SAQRGDKLSI

SEQ ID NO: 177 - protein, 12 aa, synthetic construct
  source 1..12: note = AV9ML018 VP1's amino acid sequence corresponding to position 586-588 of wild-type AAV9 VP1 plus insert peptide
SAQLNTNNSFTK

SEQ ID NO: 178 - protein, 599 aa, wild-type adeno-associated virus serotype 9 (AAV9)
  source 1..599: note = wild-type AAV9 VP2 amino acid sequence
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 534 aa, wild-type adeno-associated virus serotype 9 (AAV9)
  source 1..534: note = wild-type AAV9 VP3 amino acid sequence
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2026/0285911 A1
(43) Pub. Date: Sep. 24, 2026

(54) VARIANT AAV9 CAPSID PROTEINS AND USES THEREOF

(71) Applicant: EXEGENESIS BIO CO., Lower Gwynedd, PA (US)

(72) Inventors: Zhenhua Wu, Lower Gwynedd, PA (US); Shuyuan Chen, Hangzhou (CN); Zhongwan Li, Hangzhou (CN); Shaoyong Li, Hopkinton, MA (US); Guojie Ye, Gainesville, FL (US)

(21) Appl. No.: 19/469,955

(22) PCT Filed: Mar. 29, 2024
(86) PCT No.: PCT/CN2024/084962

§ 371 Date: Sep. 26, 2025

(30) Foreign Application Priority Data
Mar. 31, 2023 (WO) PCT/CN2023/085648

(51) Int. Cl. C07K 14/005; A61K 38/00; A61K 48/00; C12N 15/86

(52) U.S. Cl. CPC C07K 14/005; A61K 48/0033; C12N 15/86; A61K 38/00; C12N 2750/14122; C12N 2750/14143; C12N 2750/14151

(57) ABSTRACT

Provided herein are variant adeno-associated virus serotype 9 (AAV9) capsid proteins and recombinant AAV9 particles comprising the variant AAV9 capsid proteins. Also provided herein are pharmaceutical compositions comprising the recombinant AAV9 particles, polynucleotides encoding the variant AAV9 capsid proteins, vectors and host cells comprising the polynucleotides, populations of host cells transduced by the recombinant AAV9 particles, as well as various methods using the recombinant AAV9 particles.

### CROSS REFERENCE TO RELATED APPLICATIONS

This application claims the benefit of, and priority to, PCT Application No. PCT/CN2023/085648, filed Mar. 31, 2023. The contents of these applications are incorporated herein by reference in their entireties for all purposes.

### TECHNICAL FIELD

Provided herein are variant adeno-associated virus serotype 9 (AAV9) capsid proteins and recombinant AAV9 particles comprising the variant AAV9 capsid proteins. Also provided herein are pharmaceutical compositions comprising the recombinant AAV9 particles, polynucleotides encoding the variant AAV9 capsid proteins, vectors and host cells comprising the polynucleotides, populations of host cells transduced by the recombinant AAV9 particles, as well as various methods using the recombinant AAV9 particles.

### BACKGROUND

Adeno-associated virus (AAV) is a replication-deficient parvovirus that has a non-enveloped capsid and a linear single-stranded DNA genome including two 145 nucleotide-long inverted terminal repeats (ITRs) at the termini. AAV-based gene therapies have the potential to become promising treatments for many diseases. However, low transduction efficiency in certain tissues is a technical hurdle to the clinical use of AAV-based gene therapy in many situations.

To date, there remains a need for developing AAV-mediated gene delivery systems that have enhanced tissue transduction.

### SUMMARY

In one aspect, provided herein is a variant adeno-associated virus serotype 9 (AAV9) capsid protein comprising the amino acid sequence of SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168.

In certain embodiments, the variant AAV9 capsid protein comprises the amino acid sequence of SEQ ID NO: 37. In specific embodiments, the variant AAV9 capsid protein comprises the amino acid sequence of SEQ ID NO: 6.

In certain embodiments, the variant AAV9 capsid protein comprises the amino acid sequence of SEQ ID NO: 39. In specific embodiments, the variant AAV9 capsid protein comprises the amino acid sequence of SEQ ID NO: 7.

In certain embodiments, the variant AAV9 capsid protein comprises the amino acid sequence of SEQ ID NO: 41. In specific embodiments, the variant AAV9 capsid protein comprises the amino acid sequence of SEQ ID NO: 8.

In certain embodiments, the variant AAV9 capsid protein comprises the amino acid sequence of SEQ ID NO: 43. In specific embodiments, the variant AAV9 capsid protein comprises the amino acid sequence of SEQ ID NO: 9.

In certain embodiments, the variant AAV9 capsid protein comprises the amino acid sequence of SEQ ID NO: 45. In specific embodiments, the variant AAV9 capsid protein comprises the amino acid sequence of SEQ ID NO: 10.

In certain embodiments, the variant AAV9 capsid protein comprises the amino acid sequence of SEQ ID NO: 47 and comprises the amino acid sequence of SEQ ID NO: 49. In specific embodiments, the variant AAV9 capsid protein comprises the amino acid sequence of SEQ ID NO: 11.

In certain embodiments, the variant AAV9 capsid protein comprises the amino acid sequence of SEQ ID NO: 51. In specific embodiments, the variant AAV9 capsid protein comprises the amino acid sequence of SEQ ID NO: 12.

In certain embodiments, the variant AAV9 capsid protein comprises the amino acid sequence of SEQ ID NO: 53. In specific embodiments, the variant AAV9 capsid protein comprises the amino acid sequence of SEQ ID NO: 13.

In certain embodiments, the variant AAV9 capsid protein comprises the amino acid sequence of SEQ ID NO: 55. In specific embodiments, the variant AAV9 capsid protein comprises the amino acid sequence of SEQ ID NO: 14.

In certain embodiments, the variant AAV9 capsid protein comprises the amino acid sequence of SEQ ID NO: 57. In specific embodiments, the variant AAV9 capsid protein comprises the amino acid sequence of SEQ ID NO: 15.

In certain embodiments, the variant AAV9 capsid protein comprises the amino acid sequence of SEQ ID NO: 59 and comprises the amino acid sequence of SEQ ID NO: 61. In specific embodiments, the variant AAV9 capsid protein comprises the amino acid sequence of SEQ ID NO: 16.

In certain embodiments, the variant AAV9 capsid protein comprises the amino acid sequence of SEQ ID NO: 63. In specific embodiments, the variant AAV9 capsid protein comprises the amino acid sequence of SEQ ID NO: 17.

In certain embodiments, the variant AAV9 capsid protein comprises the amino acid sequence of SEQ ID NO: 65. In specific embodiments, the variant AAV9 capsid protein comprises the amino acid sequence of SEQ ID NO: 18.

In certain embodiments, the variant AAV9 capsid protein comprises the amino acid sequence of SEQ ID NO: 67. In specific embodiments, the variant AAV9 capsid protein comprises the amino acid sequence of SEQ ID NO: 19.

In certain embodiments, the variant AAV9 capsid protein comprises the amino acid sequence of SEQ ID NO: 69. In specific embodiments, the variant AAV9 capsid protein comprises the amino acid sequence of SEQ ID NO: 20.

In certain embodiments, the variant AAV9 capsid protein comprises the amino acid sequence of SEQ ID NO: 71. In specific embodiments, the variant AAV9 capsid protein comprises the amino acid sequence of SEQ ID NO: 21.

In certain embodiments, the variant AAV9 capsid protein comprises the amino acid sequence of SEQ ID NO: 73. In specific embodiments, the variant AAV9 capsid protein comprises the amino acid sequence of SEQ ID NO: 22.

In certain embodiments, the variant AAV9 capsid protein comprises the amino acid sequence of SEQ ID NO: 75. In specific embodiments, the variant AAV9 capsid protein comprises the amino acid sequence of SEQ ID NO: 23.

In certain embodiments, the variant AAV9 capsid protein comprises the amino acid sequence of SEQ ID NO: 77. In specific embodiments, the variant AAV9 capsid protein comprises the amino acid sequence of SEQ ID NO: 24.

In certain embodiments, the variant AAV9 capsid protein comprises the amino acid sequence of SEQ ID NO: 79. In specific embodiments, the variant AAV9 capsid protein comprises the amino acid sequence of SEQ ID NO: 25.

In certain embodiments, the variant AAV9 capsid protein comprises the amino acid sequence of SEQ ID NO: 80. In specific embodiments, the variant AAV9 capsid protein comprises the amino acid sequence of SEQ ID NO: 26.

In certain embodiments, the variant AAV9 capsid protein comprises the amino acid sequence of SEQ ID NO: 81. In specific embodiments, the variant AAV9 capsid protein comprises the amino acid sequence of SEQ ID NO: 27.

In certain embodiments, the variant AAV9 capsid protein comprises the amino acid sequence of SEQ ID NO: 82. In specific embodiments, the variant AAV9 capsid protein comprises the amino acid sequence of SEQ ID NO: 28.

In certain embodiments, the variant AAV9 capsid protein comprises the amino acid sequence of SEQ ID NO: 83. In specific embodiments, the variant AAV9 capsid protein comprises the amino acid sequence of SEQ ID NO: 29.

In certain embodiments, the variant AAV9 capsid protein comprises the amino acid sequence of SEQ ID NO: 84. In specific embodiments, the variant AAV9 capsid protein comprises the amino acid sequence of SEQ ID NO: 30.

In certain embodiments, the variant AAV9 capsid protein comprises the amino acid sequence of SEQ ID NO: 85. In specific embodiments, the variant AAV9 capsid protein comprises the amino acid sequence of SEQ ID NO: 31.

In certain embodiments, the variant AAV9 capsid protein comprises the amino acid sequence of SEQ ID NO: 86. In specific embodiments, the variant AAV9 capsid protein comprises the amino acid sequence of SEQ ID NO: 32.

In certain embodiments, the variant AAV9 capsid protein comprises the amino acid sequence of SEQ ID NO: 87. In specific embodiments, the variant AAV9 capsid protein comprises the amino acid sequence of SEQ ID NO: 33.

In certain embodiments, the variant AAV9 capsid protein comprises the amino acid sequence of SEQ ID NO: 116. In specific embodiments, the variant AAV9 capsid protein comprises the amino acid sequence of SEQ ID NO: 115.

In certain embodiments, the variant AAV9 capsid protein comprises the amino acid sequence of SEQ ID NO: 118. In specific embodiments, the variant AAV9 capsid protein comprises the amino acid sequence of SEQ ID NO: 117.

In certain embodiments, the variant AAV9 capsid protein comprises the amino acid sequence of SEQ ID NO: 120. In specific embodiments, the variant AAV9 capsid protein comprises the amino acid sequence of SEQ ID NO: 119.

In certain embodiments, the variant AAV9 capsid protein comprises the amino acid sequence of SEQ ID NO: 122. In specific embodiments, the variant AAV9 capsid protein comprises the amino acid sequence of SEQ ID NO: 121.

In certain embodiments, the variant AAV9 capsid protein comprises the amino acid sequence of SEQ ID NO: 124. In specific embodiments, the variant AAV9 capsid protein comprises the amino acid sequence of SEQ ID NO: 123.

In certain embodiments, the variant AAV9 capsid protein comprises the amino acid sequence of SEQ ID NO: 126. In specific embodiments, the variant AAV9 capsid protein comprises the amino acid sequence of SEQ ID NO: 125.

In certain embodiments, the variant AAV9 capsid protein comprises the amino acid sequence of SEQ ID NO: 128. In specific embodiments, the variant AAV9 capsid protein comprises the amino acid sequence of SEQ ID NO: 127.

In certain embodiments, the variant AAV9 capsid protein comprises the amino acid sequence of SEQ ID NO: 130. In specific embodiments, the variant AAV9 capsid protein comprises the amino acid sequence of SEQ ID NO: 129.

In certain embodiments, the variant AAV9 capsid protein comprises the amino acid sequence of SEQ ID NO: 132. In specific embodiments, the variant AAV9 capsid protein comprises the amino acid sequence of SEQ ID NO: 131.

In certain embodiments, the variant AAV9 capsid protein comprises the amino acid sequence of SEQ ID NO: 134. In specific embodiments, the variant AAV9 capsid protein comprises the amino acid sequence of SEQ ID NO: 133.

In certain embodiments, the variant AAV9 capsid protein comprises the amino acid sequence of SEQ ID NO: 136. In specific embodiments, the variant AAV9 capsid protein comprises the amino acid sequence of SEQ ID NO: 135.

In certain embodiments, the variant AAV9 capsid protein comprises the amino acid sequence of SEQ ID NO: 138. In specific embodiments, the variant AAV9 capsid protein comprises the amino acid sequence of SEQ ID NO: 137.

In certain embodiments, the variant AAV9 capsid protein comprises the amino acid sequence of SEQ ID NO: 140. In specific embodiments, the variant AAV9 capsid protein comprises the amino acid sequence of SEQ ID NO: 139.

In certain embodiments, the variant AAV9 capsid protein comprises the amino acid sequence of SEQ ID NO: 142. In specific embodiments, the variant AAV9 capsid protein comprises the amino acid sequence of SEQ ID NO: 141.

In certain embodiments, the variant AAV9 capsid protein comprises the amino acid sequence of SEQ ID NO: 144. In specific embodiments, the variant AAV9 capsid protein comprises the amino acid sequence of SEQ ID NO: 143.

In certain embodiments, the variant AAV9 capsid protein comprises the amino acid sequence of SEQ ID NO: 146. In specific embodiments, the variant AAV9 capsid protein comprises the amino acid sequence of SEQ ID NO: 145.

In certain embodiments, the variant AAV9 capsid protein comprises the amino acid sequence of SEQ ID NO: 148. In specific embodiments, the variant AAV9 capsid protein comprises the amino acid sequence of SEQ ID NO: 147.

In certain embodiments, the variant AAV9 capsid protein comprises the amino acid sequence of SEQ ID NO: 150. In specific embodiments, the variant AAV9 capsid protein comprises the amino acid sequence of SEQ ID NO: 149.

In certain embodiments, the variant AAV9 capsid protein comprises the amino acid sequence of SEQ ID NO: 152. In specific embodiments, the variant AAV9 capsid protein comprises the amino acid sequence of SEQ ID NO: 151.

In certain embodiments, the variant AAV9 capsid protein comprises the amino acid sequence of SEQ ID NO: 154. In specific embodiments, the variant AAV9 capsid protein comprises the amino acid sequence of SEQ ID NO: 153.

In certain embodiments, the variant AAV9 capsid protein comprises the amino acid sequence of SEQ ID NO: 156. In specific embodiments, the variant AAV9 capsid protein comprises the amino acid sequence of SEQ ID NO: 155.

In certain embodiments, the variant AAV9 capsid protein comprises the amino acid sequence of SEQ ID NO: 158. In specific embodiments, the variant AAV9 capsid protein comprises the amino acid sequence of SEQ ID NO: 157.

In certain embodiments, the variant AAV9 capsid protein comprises the amino acid sequence of SEQ ID NO: 160. In specific embodiments, the variant AAV9 capsid protein comprises the amino acid sequence of SEQ ID NO: 159.

In certain embodiments, the variant AAV9 capsid protein comprises the amino acid sequence of SEQ ID NO: 162. In specific embodiments, the variant AAV9 capsid protein comprises the amino acid sequence of SEQ ID NO: 161.

In certain embodiments, the variant AAV9 capsid protein comprises the amino acid sequence of SEQ ID NO: 164. In specific embodiments, the variant AAV9 capsid protein comprises the amino acid sequence of SEQ ID NO: 163.

In certain embodiments, the variant AAV9 capsid protein comprises the amino acid sequence of SEQ ID NO: 166. In specific embodiments, the variant AAV9 capsid protein comprises the amino acid sequence of SEQ ID NO: 165.

In certain embodiments, the variant AAV9 capsid protein comprises the amino acid sequence of SEQ ID NO: 168. In specific embodiments, the variant AAV9 capsid protein comprises the amino acid sequence of SEQ ID NO: 167.

In various embodiments, a variant AAV9 capsid protein described herein is associated with an increased tropism for liver relative to a wild-type AAV9 capsid protein comprising the amino acid sequence of SEQ ID NO: 2.

In various embodiments, a variant AAV9 capsid protein described herein is associated with an increased tropism for brain relative to a wild-type AAV9 capsid protein comprising the amino acid sequence of SEQ ID NO: 2.

In various embodiments, a variant AAV9 capsid protein described herein is associated with an increased tropism for muscle relative to a wild-type AAV9 capsid protein comprising the amino acid sequence of SEQ ID NO: 2. In certain embodiments, a variant AAV9 capsid protein described herein is associated with an increased tropism for skeletal muscle and/or heart muscle relative to the wild-type AAV9 capsid protein. In certain embodiments, a variant AAV9 capsid protein described herein is associated with an increased tropism for biceps, triceps, quadriceps, tibialis anterior, gastrocnemius, rectus abdominis, diaphragm, pectoralis major, heart atrium muscle, and/or heart ventricle muscle relative to the wild-type AAV9 capsid protein.

In another aspect, provided herein is a recombinant AAV9 particle comprising a variant AAV9 capsid protein described herein.

In another aspect, provided herein is a pharmaceutical composition comprising a recombinant AAV9 particle described herein and a pharmaceutically acceptable carrier.

In another aspect, provided herein is a polynucleotide encoding a variant AAV9 capsid protein described herein.

In another aspect, provided herein is a vector comprising a polynucleotide described herein.

In another aspect, provided herein is a host cell comprising a polynucleotide described herein or a vector described herein.

In another aspect, provided herein is a population of host cells stably transduced by a recombinant AAV9 particle described herein.

In another aspect, provided herein is a pharmaceutical composition comprising a population of host cells described herein and a pharmaceutically acceptable carrier.

In another aspect, provided herein is a method of delivering a biologic molecule to one or more ex vivo or in vitro target cells, comprising transducing the one or more target cells with a recombinant AAV9 particle described herein. In certain embodiments, the one or more target cells are one or more muscle cells, one or more liver cells, and/or one or more brain cells.

In another aspect, provide herein is a method of delivering a biologic molecule to one or more in vivo target cells in a subject, comprising administering to the subject a recombinant AAV9 particle described herein or a pharmaceutical composition described herein comprising a recombinant AAV9 particle and a pharmaceutically acceptable carrier. In certain embodiments, the one or more target cells are one or more muscle cells, one or more liver cells, and/or one or more brain cells. In certain embodiments, the subject is a human.

In another aspect, provide herein is a method of treating a disease or disorder in a subject in need thereof, comprising administering to the subject a recombinant AAV9 particle described herein or a pharmaceutical composition described herein. In certain embodiments, the subject is a human.

In another aspect, provide herein is a method of producing a recombinant AAV9 particle, comprising culturing a host cell described herein.

### ILLUSTRATIVE EMBODIMENTS

The present disclosure includes the following non-limiting illustrative embodiments:

1. A variant adeno-associated virus serotype 9 (AAV9) capsid protein comprising the amino acid sequence of SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168.

2. The variant AAV9 capsid protein of embodiment 1, which comprises the amino acid sequence of SEQ ID NO: 37.

3. The variant AAV9 capsid protein of embodiment 2, which comprises the amino acid sequence of SEQ ID NO: 6.

4. The variant AAV9 capsid protein of embodiment 1, which comprises the amino acid sequence of SEQ ID NO: 39.

5. The variant AAV9 capsid protein of embodiment 4, which comprises the amino acid sequence of SEQ ID NO: 7.

6. The variant AAV9 capsid protein of embodiment 1, which comprises the amino acid sequence of SEQ ID NO: 41.

7. The variant AAV9 capsid protein of embodiment 6, which comprises the amino acid sequence of SEQ ID NO: 8.

8. The variant AAV9 capsid protein of embodiment 1, which comprises the amino acid sequence of SEQ ID NO: 43.

9. The variant AAV9 capsid protein of embodiment 8, which comprises the amino acid sequence of SEQ ID NO: 9.

10. The variant AAV9 capsid protein of embodiment 1, which comprises the amino acid sequence of SEQ ID NO: 45.

11. The variant AAV9 capsid protein of embodiment 10, which comprises the amino acid sequence of SEQ ID NO: 10.

12. The variant AAV9 capsid protein of embodiment 1, which comprises the amino acid sequence of SEQ ID NO: 47 and comprises the amino acid sequence of SEQ ID NO: 49.

13. The variant AAV9 capsid protein of embodiment 12, which comprises the amino acid sequence of SEQ ID NO: 11.

14. The variant AAV9 capsid protein of embodiment 1, which comprises the amino acid sequence of SEQ ID NO: 51.

15. The variant AAV9 capsid protein of embodiment 14, which comprises the amino acid sequence of SEQ ID NO: 12.

16. The variant AAV9 capsid protein of embodiment 1, which comprises the amino acid sequence of SEQ ID NO: 53.

17. The variant AAV9 capsid protein of embodiment 16, which comprises the amino acid sequence of SEQ ID NO: 13.

18. The variant AAV9 capsid protein of embodiment 1, which comprises the amino acid sequence of SEQ ID NO: 55.

19. The variant AAV9 capsid protein of embodiment 18, which comprises the amino acid sequence of SEQ ID NO: 14.

20. The variant AAV9 capsid protein of embodiment 1, which comprises the amino acid sequence of SEQ ID NO: 57.

21. The variant AAV9 capsid protein of embodiment 20, which comprises the amino acid sequence of SEQ ID NO: 15.

22. The variant AAV9 capsid protein of embodiment 1, which comprises the amino acid sequence of SEQ ID NO: 59 and comprises the amino acid sequence of SEQ ID NO: 61.

23. The variant AAV9 capsid protein of embodiment 22, which comprises the amino acid sequence of SEQ ID NO: 16.

24. The variant AAV9 capsid protein of embodiment 1, which comprises the amino acid sequence of SEQ ID NO: 63.

25. The variant AAV9 capsid protein of embodiment 24, which comprises the amino acid sequence of SEQ ID NO: 17.

26. The variant AAV9 capsid protein of embodiment 1, which comprises the amino acid sequence of SEQ ID NO: 65.

27. The variant AAV9 capsid protein of embodiment 26, which comprises the amino acid sequence of SEQ ID NO: 18.

28. The variant AAV9 capsid protein of embodiment 1, which comprises the amino acid sequence of SEQ ID NO: 67.

29. The variant AAV9 capsid protein of embodiment 28, which comprises the amino acid sequence of SEQ ID NO: 19.

30. The variant AAV9 capsid protein of embodiment 1, which comprises the amino acid sequence of SEQ ID NO: 69.

31. The variant AAV9 capsid protein of embodiment 30, which comprises the amino acid sequence of SEQ ID NO: 20.

32. The variant AAV9 capsid protein of embodiment 1, which comprises the amino acid sequence of SEQ ID NO: 71.

33. The variant AAV9 capsid protein of embodiment 32, which comprises the amino acid sequence of SEQ ID NO: 21.

34. The variant AAV9 capsid protein of embodiment 1, which comprises the amino acid sequence of SEQ ID NO: 73.

35. The variant AAV9 capsid protein of embodiment 34, which comprises the amino acid sequence of SEQ ID NO: 22.

36. The variant AAV9 capsid protein of embodiment 1, which comprises the amino acid sequence of SEQ ID NO: 75.

37. The variant AAV9 capsid protein of embodiment 36, which comprises the amino acid sequence of SEQ ID NO: 23.

38. The variant AAV9 capsid protein of embodiment 1, which comprises the amino acid sequence of SEQ ID NO: 77.

39. The variant AAV9 capsid protein of embodiment 38, which comprises the amino acid sequence of SEQ ID NO: 24.

40. The variant AAV9 capsid protein of embodiment 1, which comprises the amino acid sequence of SEQ ID NO: 79.

41. The variant AAV9 capsid protein of embodiment 40, which comprises the amino acid sequence of SEQ ID NO: 25.

42. The variant AAV9 capsid protein of embodiment 1, which comprises the amino acid sequence of SEQ ID NO: 80.

43. The variant AAV9 capsid protein of embodiment 42, which comprises the amino acid sequence of SEQ ID NO: 26.

44. The variant AAV9 capsid protein of embodiment 1, which comprises the amino acid sequence of SEQ ID NO: 81.

45. The variant AAV9 capsid protein of embodiment 44, which comprises the amino acid sequence of SEQ ID NO: 27.

46. The variant AAV9 capsid protein of embodiment 1, which comprises the amino acid sequence of SEQ ID NO: 82.

47. The variant AAV9 capsid protein of embodiment 46, which comprises the amino acid sequence of SEQ ID NO: 28.

48. The variant AAV9 capsid protein of embodiment 1, which comprises the amino acid sequence of SEQ ID NO: 83.

49. The variant AAV9 capsid protein of embodiment 48, which comprises the amino acid sequence of SEQ ID NO: 29.

50. The variant AAV9 capsid protein of embodiment 1, which comprises the amino acid sequence of SEQ ID NO: 84.

51. The variant AAV9 capsid protein of embodiment 50, which comprises the amino acid sequence of SEQ ID NO: 30.

52. The variant AAV9 capsid protein of embodiment 1, which comprises the amino acid sequence of SEQ ID NO: 85.

53. The variant AAV9 capsid protein of embodiment 52, which comprises the amino acid sequence of SEQ ID NO: 31.

54. The variant AAV9 capsid protein of embodiment 1, which comprises the amino acid sequence of SEQ ID NO: 86.

55. The variant AAV9 capsid protein of embodiment 54, which comprises the amino acid sequence of SEQ ID NO: 32.

56. The variant AAV9 capsid protein of embodiment 1, which comprises the amino acid sequence of SEQ ID NO: 87.

57. The variant AAV9 capsid protein of embodiment 56, which comprises the amino acid sequence of SEQ ID NO: 33.

58. The variant AAV9 capsid protein of embodiment 1, which comprises the amino acid sequence of SEQ ID NO: 116.

59. The variant AAV9 capsid protein of embodiment 58, which comprises the amino acid sequence of SEQ ID NO: 115.

60. The variant AAV9 capsid protein of embodiment 1, which comprises the amino acid sequence of SEQ ID NO: 118.

61. The variant AAV9 capsid protein of embodiment 60, which comprises the amino acid sequence of SEQ ID NO: 117.

62. The variant AAV9 capsid protein of embodiment 1, which comprises the amino acid sequence of SEQ ID NO: 120.

63. The variant AAV9 capsid protein of embodiment 62, which comprises the amino acid sequence of SEQ ID NO: 119.

64. The variant AAV9 capsid protein of embodiment 1, which comprises the amino acid sequence of SEQ ID NO: 122.

65. The variant AAV9 capsid protein of embodiment 64, which comprises the amino acid sequence of SEQ ID NO: 121.

66. The variant AAV9 capsid protein of embodiment 1, which comprises the amino acid sequence of SEQ ID NO: 124.

67. The variant AAV9 capsid protein of embodiment 66, which comprises the amino acid sequence of SEQ ID NO: 123.

68. The variant AAV9 capsid protein of embodiment 1, which comprises the amino acid sequence of SEQ ID NO: 126.

69. The variant AAV9 capsid protein of embodiment 68, which comprises the amino acid sequence of SEQ ID NO: 125.

70. The variant AAV9 capsid protein of embodiment 1, which comprises the amino acid sequence of SEQ ID NO: 128.

71. The variant AAV9 capsid protein of embodiment 70, which comprises the amino acid sequence of SEQ ID NO: 127.

72. The variant AAV9 capsid protein of embodiment 1, which comprises the amino acid sequence of SEQ ID NO: 130.

73. The variant AAV9 capsid protein of embodiment 72, which comprises the amino acid sequence of SEQ ID NO: 129.

74. The variant AAV9 capsid protein of embodiment 1, which comprises the amino acid sequence of SEQ ID NO: 132.

75. The variant AAV9 capsid protein of embodiment 74, which comprises the amino acid sequence of SEQ ID NO: 131.

76. The variant AAV9 capsid protein of embodiment 1, which comprises the amino acid sequence of SEQ ID NO: 134.

77. The variant AAV9 capsid protein of embodiment 76, which comprises the amino acid sequence of SEQ ID NO: 133.

78. The variant AAV9 capsid protein of embodiment 1, which comprises the amino acid sequence of SEQ ID NO: 136.

79. The variant AAV9 capsid protein of embodiment 78, which comprises the amino acid sequence of SEQ ID NO: 135.

80. The variant AAV9 capsid protein of embodiment 1, which comprises the amino acid sequence of SEQ ID NO: 138.

81. The variant AAV9 capsid protein of embodiment 80, which comprises the amino acid sequence of SEQ ID NO: 137.

82. The variant AAV9 capsid protein of embodiment 1, which comprises the amino acid sequence of SEQ ID NO: 140.

83. The variant AAV9 capsid protein of embodiment 82, which comprises the amino acid sequence of SEQ ID NO: 139.

84. The variant AAV9 capsid protein of embodiment 1, which comprises the amino acid sequence of SEQ ID NO: 142.

85. The variant AAV9 capsid protein of embodiment 84, which comprises the amino acid sequence of SEQ ID NO: 141.

86. The variant AAV9 capsid protein of embodiment 1, which comprises the amino acid sequence of SEQ ID NO: 144.

87. The variant AAV9 capsid protein of embodiment 86, which comprises the amino acid sequence of SEQ ID NO: 143.

88. The variant AAV9 capsid protein of embodiment 1, which comprises the amino acid sequence of SEQ ID NO: 146.

89. The variant AAV9 capsid protein of embodiment 88, which comprises the amino acid sequence of SEQ ID NO: 145.

90. The variant AAV9 capsid protein of embodiment 1, which comprises the amino acid sequence of SEQ ID NO: 148.

91. The variant AAV9 capsid protein of embodiment 90, which comprises the amino acid sequence of SEQ ID NO: 147.

92. The variant AAV9 capsid protein of embodiment 1, which comprises the amino acid sequence of SEQ ID NO: 150.

93. The variant AAV9 capsid protein of embodiment 92, which comprises the amino acid sequence of SEQ ID NO: 149.

94. The variant AAV9 capsid protein of embodiment 1, which comprises the amino acid sequence of SEQ ID NO: 152.

95. The variant AAV9 capsid protein of embodiment 94, which comprises the amino acid sequence of SEQ ID NO: 151.

96. The variant AAV9 capsid protein of embodiment 1, which comprises the amino acid sequence of SEQ ID NO: 154.

97. The variant AAV9 capsid protein of embodiment 96, which comprises the amino acid sequence of SEQ ID NO: 153.

98. The variant AAV9 capsid protein of embodiment 1, which comprises the amino acid sequence of SEQ ID NO: 156.

99. The variant AAV9 capsid protein of embodiment 98, which comprises the amino acid sequence of SEQ ID NO: 155.

100. The variant AAV9 capsid protein of embodiment 1, which comprises the amino acid sequence of SEQ ID NO: 158.

101. The variant AAV9 capsid protein of embodiment 100, which comprises the amino acid sequence of SEQ ID NO: 157.

102. The variant AAV9 capsid protein of embodiment 1, which comprises the amino acid sequence of SEQ ID NO: 160.

103. The variant AAV9 capsid protein of embodiment 102, which comprises the amino acid sequence of SEQ ID NO: 159.

104. The variant AAV9 capsid protein of embodiment 1, which comprises the amino acid sequence of SEQ ID NO: 162.

105. The variant AAV9 capsid protein of embodiment 104, which comprises the amino acid sequence of SEQ ID NO: 161.

106. The variant AAV9 capsid protein of embodiment 1, which comprises the amino acid sequence of SEQ ID NO: 164.

107. The variant AAV9 capsid protein of embodiment 106, which comprises the amino acid sequence of SEQ ID NO: 163.

108. The variant AAV9 capsid protein of embodiment 1, which comprises the amino acid sequence of SEQ ID NO: 166.

109. The variant AAV9 capsid protein of embodiment 108, which comprises the amino acid sequence of SEQ ID NO: 165.

110. The variant AAV9 capsid protein of embodiment 1, which comprises the amino acid sequence of SEQ ID NO: 168.

111. The variant AAV9 capsid protein of embodiment 110, which comprises the amino acid sequence of SEQ ID NO: 167.

112. The variant AAV9 capsid protein of any one of embodiments 8-9, 14-15 and 24-35, which is associated with an increased tropism for liver relative to a wild-type AAV9 capsid protein comprising the amino acid sequence of SEQ ID NO: 2.

113. The variant AAV9 capsid protein of any one of embodiments 24-25, 30-31, 34-37, 42-43 and 46-47, which is associated with an increased tropism for brain relative to a wild-type AAV9 capsid protein comprising the amino acid sequence of SEQ ID NO: 2.

114. The variant AAV9 capsid protein of any one of embodiments 1-113, which is associated with an increased tropism for muscle relative to a wild-type AAV9 capsid protein comprising the amino acid sequence of SEQ ID NO: 2.

115. The variant AAV9 capsid protein of embodiment 114, which is associated with an increased tropism for skeletal muscle and/or heart muscle relative to the wild-type AAV9 capsid protein.

116. The variant AAV9 capsid protein of embodiment 114, which is associated with an increased tropism for biceps, triceps, quadriceps, tibialis anterior, gastrocnemius, rectus abdominis, diaphragm, pectoralis major, heart atrium muscle, and/or heart ventricle muscle relative to the wild-type AAV9 capsid protein.

117. A recombinant AAV9 particle comprising the variant AAV9 capsid protein of any one of embodiments 1-116.

118. A pharmaceutical composition comprising the recombinant AAV9 particle of embodiment 117 and a pharmaceutically acceptable carrier.

119. A polynucleotide encoding the variant AAV9 capsid protein of any one of embodiments 1-116.

120. A vector comprising the polynucleotide of embodiment 119.

121. A host cell comprising the polynucleotide of embodiment 119 or the vector of embodiment 120.

122. A population of host cells stably transduced by the recombinant AAV9 particle of embodiment 117.

123. A pharmaceutical composition comprising the population of host cells of embodiment 122 and a pharmaceutically acceptable carrier.

124. A method of delivering a biologic molecule to one or more ex vivo or in vitro target cells, comprising transducing the one or more target cells with the recombinant AAV9 particle of embodiment 117.

125. A method of delivering a biologic molecule to one or more in vivo target cells in a subject, comprising administering to the subject the recombinant AAV9 particle of embodiment 117 or the pharmaceutical composition of embodiment 118.

126. The method of embodiment 124 or 125, wherein the one or more target cells are one or more muscle cells, one or more liver cells, and/or one or more brain cells.

127. A method of treating a disease or disorder in a subject in need thereof, comprising administering to the subject the recombinant AAV9 particle of embodiment 117 or the pharmaceutical composition of embodiment 118 or 123.

128. The method of any one of embodiments 125-127, wherein the subject is a human.

129. A method of producing a recombinant AAV9 particle, comprising culturing the host cell of embodiment 121.

### BRIEF DESCRIPTION OF THE FIGURES

FIGS. 1A-1D. AAV library design. FIG. 1A: Library #1. FIG. 1B: Library #2. FIG. 1C: Library #3. FIG. 1D: Library #4. ITR: inverted terminal repeat; p41: AAV5 p41 promoter; X: any amino acid; pA: poly A signal.

FIG. 2. AAV library screening workflow.

FIG. 3. In vivo transduction comparison by imaging. Eight-week-old C57BL/6J mice were intravenously injected with AAV containing CBA-GFP transgene (1e12 vg/mouse). CBA: Chicken β-actin promote; GFP: green fluorescent protein. Two weeks post injection (wpi), the ex vivo imaging was used to detect GFP expression. MyoAAV 1A (1A) and wild-type AAV9 were used as controls. Tri: Triceps; Quad: Quadriceps; TA: Tibialis anterior; GAS: Gastrocnemius; H: Heart; L: Liver; Sp: Spleen; Lung: Lung; Ki: Kidney.

FIG. 4. In vivo transduction comparison by RT-PCR. Eight-week-old C57BL/6J mice were intravenously injected with AAV containing CBA-GFP transgene (1e12 vg/mouse). CBA: Chicken β-actin promote; GFP: green fluorescent protein. Two weeks post injection (wpi), the total RNA was isolated from tissues and proceeded to RT-qPCR for GFP expression quantification. MyoAAV 1A (1A) and wild-type AAV9 were used as controls. The GFP expression levels (fold change over wild-type AAV9) for each variant in the tested tissues are: AVT901 (Tri: 6.4; Q: 5.7; TA: 4.4; GAS: 5.3; H: 1.7; liver: 1.2; S: 0.6; lung: 0.7; K: 1.4); AVT903 (Tri: 2.5; Q: 1.8; TA: 1.6; GAS: 2.2; H: 0.9; liver: 0.9; S: 0.4; lung: 0.7; K: 0.9); AVT905 (Tri: 11.6; Q: 11.5; TA: 3.4; GAS: 6.1; H: 2.2; liver: 2.7; S: 3.1; lung: 5.3; K: 1.0); AVT906 (Tri: 0.6; Q: 0.9; TA: 0.3; GAS: 0.6; H: 0.8; liver: 1.1; S: 0.6; lung: 0.7; K: 0.8); AVT907 (Tri: 36.9; Q: 28.2; TA: 16.5; GAS: 35.2; H: 9.7; liver: 0.3; S: 0.3; lung: 1.1; K: 0.3); Myo1A (Tri: 22.3; Q: 16.8; TA: 16.3; GAS: 27.7; H: 7.5; liver: 0.6; S: 0.7; lung: 1.2; K: 1.1). Myo1A: MyoAAV 1A. Values in Triceps (Tri), Quadriceps (Q), Tibialis anterior (TA), Gastrocnemius (GAS), Heart (H), liver, Spleen (S), lung and Kidney (K) are displayed from left to right for each variant.

FIG. 5. Vector genome copy analysis. Eight-week-old C57BL/6J mice were intravenously injected with AAV containing CBA-GFP transgene (1e12 vg/mouse). CBA: Chicken β-actin promote; GFP: green fluorescent protein. Two weeks post injection (wpi), the genomic DNA was isolated from tissues and proceed to ddPCR for AAV vector genome copy analysis. MyoAAV 1A (1A) and wild-type AAV9 were used as controls. The folds of genome copy over wild-type AAV9 for each AAV9 variant in the tested tissues are: AVT901 (Tri: 2.6; Q: 2.6; TA: 1.4; GAS: 1.6; H: 1.3; S: 3.8; lung: 1.1; K: 0.9; L: 0.6); AVT903 (Tri: 2.2; Q: 2.8; TA: 1.1; GAS: 0.8; H: 1.9; S: 2.6; lung: 0.7; K: 0.9; L: 0.3); AVT905 (Tri: 3.9; Q: 10.9; TA: 3.0; GAS: 6.8; H: 5.0; S: 1.3; lung: 12.2; K: 3.0; L: 3.7); AVT906 (Tri: 4.9; Q: 1.8; TA: no data; GAS: 0.4; H: 1.3; S: 1.7; lung: 1.0; K: 0.8; L: 0.8); AVT907 (Tri: 7.1; Q: 5.4; TA: 1.9; GAS: 5.9; H: 10.0; S: 2.2; lung: 2.0; K: 1.0; L: 0.2); Myo1A (Tri: 7.7; Q: 7.8; TA: 2.8; GAS: 5.1; H: 6.0; S: 2.3; lung: 2.3; K: 2.0; L: 0.5). Values in Triceps (Tri), Quadriceps (Q), Tibialis anterior (TA), Gastrocnemius (GAS), Heart (H), Spleen (S), Lung, Kidney (K), and Liver (L) are displayed from left to right for each variant. Myo1A: MyoAAV 1A.

FIGS. 6A and 6B. In vitro transduction comparison by imaging. FIG. 6A: C2C12 myoblast cells were seeded into 96-well plates and incubated (1e6 vg/cell) with indicated AAV vectors containing CB-Fluc-2A-GFP transgene. Twenty-four hours after transduction, the native GFP imaging was captured with same conditions. FIG. 6B: HepG2 cells were seeded into 96-well plates and incubated (1e6 vg/cell) with indicated AAV vectors containing CB-Fluc-2A-GFP transgene. Twenty-four hours after transduction, the native GFP imaging was captured with same conditions. CB: Chicken β-actin promoter; Fluc: Firefly luciferase; GFP: green fluorescent protein. MyoAAV 1A (1A), MyoAAV 2A (2A), MyoAAV 4A (4A) and wild-type AAV9 were used as controls.

FIGS. 7A and 7B. In vitro transduction comparison by luciferase assay. FIG. 7A: C2C12 myoblast cells were seeded into 96-well plates and incubated (1e6 vg/cell) with indicated AAV vectors containing CB-Fluc-2A-GFP transgene. Forty-eight hours after transduction, the luciferase assay was performed for quantification. FIG. 7B: HepG2 cells were seeded into 96-well plates and incubated (1e6 vg/cell) with indicated AAV vectors containing CB-Fluc-2A-GFP transgene. Forty-eight hours after transduction, the luciferase assay was performed for quantification. CB: Chicken β-actin promoter; Flue: Firefly luciferase; GFP: green fluorescent protein. MyoAAV 1A (1A), MyoAAV 2A (2A), MyoAAV 4A (4A) and wild-type AAV9 (AV9) were used as controls.

FIG. 8. Transduction efficiency comparison by in vivo imaging system (IVIS). Eight-week-old BALB/c mice were intravenously injected with AAV containing CB-Fluc-2A-GFP transgene (4e11 vg/mouse). Three weeks post injection (wpi), the IVIS was used to detect luciferase expression. MyoAAV 1A, MyoAAV 2A, MyoAAV 4A and wild-type AAV9 were used as controls.

FIGS. 9A-9I. In vivo transduction efficiency comparison. Tri: Triceps; Q: Quadriceps; TA: Tibialis anterior; GAS: Gastrocnemius; H: Heart; L: Liver. FIG. 9A: In vivo transduction efficiency comparison by RT-qPCR. Eight-week-old BALB/c mice were intravenously injected with AAV containing CB-Fluc-2A-GFP transgene (4e11 vg/mouse). Three weeks post injection (wpi), the total RNA was isolated from tissues and proceeded to RT-qPCR for transgene expression quantification. MyoAAV 1A, MyoAAV 2A, MyoAAV 4A and wild-type AAV9 were used as controls. The transgene expression levels (normalized to GAPDH) for each variant in the tested tissues are: AVT901 (T: 0.1; Q: 0.3; TA: 0.1; GAS: 0.1; H: 0.1; L: 0.1); AVT902 (T: 0.05; Q: 0.1; TA: 0.037; GAS: 0.1; H: 0.3; L: 0.2); AVT907 (T: 0.3; Q: 0.5; TA: 0.2; GAS: 0.6; H: 1.2; L: 0.1); AVT908 (T: 0.3; Q: 0.9; TA: 0.2; GAS: 1.1; H: 0.2; L: 0.8); AVT909 (T: 0.1; Q: 0.2; TA: 0.1; GAS: 0.2; H: 0.1; L: 0.1); AVT910 (T: 0.2; Q: 0.3; TA: 0.024; GAS: 0.2; H: 0.1; L: 0.2); AVT913 (T: 1.3; Q: 1.6; TA: 0.7; GAS: 2.3; H: 2.5; L: 0.1); AVT914 (T: 1.0; Q: 0.7; TA: 0.4; GAS: 0.6; H: 0.8; L: 0.2); AVT915 (T: 0.5; Q: 0.8; TA: 0.2; GAS: 1.2; H: 0.5; L: 0.1); AVT916 (T: 0.7; Q: 0.7; TA: 0.3; GAS: 0.8; H: 0.7; L: 0.1); AVT917 (T: 0.5; Q: 0.6; TA: 0.3; GAS: 0.9; H: 0.7; L: 0.1); AVT918 (T: 0.6; Q: 0.5; TA: 0.3; GAS: 0.6; H: 0.4; L: 0.1); AVT919 (T: 1.5; Q: 2.2; TA: 1.1; GAS: 2.3; H: 1.5; L: 0.2); AVT920 (T: 0.9; Q: 1.0; TA: 0.5; GAS: 0.9; H: 0.8; L: 0.1); MyoAAV 1A (T: 1.0; Q: 1.1; TA: 0.5; GAS: 1.4; H: 1.0; L: 0.1); MyoAAV 2A (T: 0.4; Q: 0.7; TA: 0.2; GAS: 0.8; H: 0.9; L: 0.1); MyoAAV 4A (T: 0.4; Q: 0.6; TA: 0.2; GAS: 0.8; H: 0.6; L: 0.1); AAV9 (T: 0.021; Q: 0.049; TA: 0.0095; GAS: 0.027; H: 0.1; L: 0.3). Values in Triceps (T), Quadriceps (Q), Tibialis anterior (TA), Gastrocnemius (GAS), Heart (H), and Liver (L) are displayed from left to right for each variant. FIG. 9B: In vivo transduction efficiency comparison (fold change over wild-type AAV9). Eight-week-old BALB/c mice were intravenously injected with AAV containing CB-Fluc-2A-GFP transgene (4e11 vg/mouse). Three weeks post injection (wpi), the total RNA was isolated from tissues and proceeded to RT-qPCR for transgene expression quantification. The folds of transgene expression levels for each variant relative to wild-type AAV9 in tested tissues are: AVT901 (T: 5.7; Q: 5.2; TA: 8.8; GAS: 5.2; H: 1.4; L: 0.4); AVT902 (T: 2.4; Q: 2.1; TA: 3.9; GAS: 2.5; H: 3.2; L: 0.5); AVT907 (T: 14.5; Q: 10.8; TA: 17.6; GAS: 21.1; H: 12.7; L: 0.2); AVT908 (T: 14.9; Q: 19.3; TA: 17.7; GAS: 39.9; H: 2.3; L: 2.7); AVT909 (T: 6.3; Q: 3.2; TA: 8.0; GAS: 6.4; H: 1.6; L: 0.4); AVT910 (T: 7.9; Q: 6.2; TA: 2.5; GAS: 7.6; H: 1.0; L: 0.7); AVT913 (T: 61.8; Q: 32.6; TA:75.2; GAS: 83.7; H: 26.7; L: 0.3); AVT914 (T: 48.6; Q: 14.9; TA: 45.0; GAS: 21.4; H: 8.5; L: 0.6); AVT915 (T: 23.4; Q: 16.5; TA: 26.2; GAS: 44.5; H: 5.6; L: 0.3); AVT916 (T: 33.8; Q: 14.4; TA: 28.5; GAS: 29.9; H: 7.1; L: 0.3); AVT917 (T: 23.3; Q: 11.9; TA: 36.3; GAS: 31.1; H: 6.9; L: 0.2); AVT918 (T: 27.4; Q: 11.2; TA: 26.4; GAS: 23.7; H: 3.8; L: 0.4); AVT919 (T: 71.6; Q: 44.8; TA: 113.8; GAS: 85.0; H: 15.3; L: 0.6); AVT920 (T: 42.5; Q: 20.3; TA: 54.7; GAS: 32.4; H: 8.4; L: 0.2); MyoAAV 1A (T: 47.4; Q: 22.1; TA: 51.9; GAS: 49.7; H: 10.0; L: 0.4); MyoAAV 2A (T: 20.6; Q: 15.3; TA: 20.3; GAS: 28.9; H: 9.2; L: 0.2); MyoAAV 4A (T: 19.5; Q: 12.9; TA: 23.0; GAS: 29.3; H: 6.7; L: 0.4); AAV9 (T: 1.0; Q: 1.0; TA: 1.0; GAS: 1.0; H: 1.0; L: 1.0). Values in Triceps (T), Quadriceps (Q), Tibialis anterior (TA), Gastrocnemius (GAS), Heart (H), and Liver (L) are displayed from left to right for each variant. FIG. 9C: In vivo transduction efficiency of the top 3 variants shown in FIG. 9B. FIG. 9D-9I: Folds of transgene expression levels for various variants relative to wild-type AAV9 in Triceps (FIG. 9D), Quadriceps (FIG. 9E), Tibialis anterior (FIG. 9F), Gastrocnemius (FIG. 9G), Heart (FIG. 9H), and Liver (FIG. 9I). Eight-week-old BALB/c mice were intravenously injected with AAV containing CB-Fluc-2A-GFP transgene (4e11 vg/mouse). Three weeks post injection (wpi), the total RNA was isolated from tissues and proceeded to RT-qPCR for transgene expression quantification. *p<0.05, **p<0.01, ***p<0.001, ****p<0.0001, one-way ANOVA, Tukey's multiple comparisons test. Variant AVT919 showed superior skeletal muscle transduction. Variant AVT913 showed superior heart transduction. Variant AVT908 showed improved transduction in both liver and muscle compared to wild-type AAV9.

FIG. 10. AAV vector genome copies in mouse tissues. Eight-week-old BALB/c mice were intravenously injected with AAV containing CB-Fluc-2A-GFP transgene (4e11 vg/mouse). Three weeks post injection (wpi), the genomic DNA was isolated from tissues and proceeded to ddPCR. Mouse TFRC and luciferase primers/probes were used. Quads: Quadriceps; TA: Tibialis anterior; Gas: Gastrocnemius. MyoAAV 1A, MyoAAV 2A, MyoAAV 4A and wild-type AAV9 were used as controls. The folds of AAV vector genome copies of various variants relative to wild-type AAV9 in tested tissues are: Triceps (AVT908: 6.8; AVT913: 7.6; AVT919: 22.1; MyoAAV2A: 5.1; AAV9: 1.0); Quadriceps (AVT908: 4.8; AVT913: 2.3; AVT919: 10.7; MyoAAV2A: 2.6; AAV9: 1.0); Tibialis anterior (AVT908: 6.6; AVT913: 8.0; AVT919: 22.4; MyoAAV2A: 4.4; AAV9: 1.0); Gastrocnemius (AVT908: 9.5; AVT913: 9.4; AVT919: 13.7; MyoAAV2A: 5.7; AAV9: 1.0); Heart (AVT908: 3.1; AVT913: 11.4; AVT919: 13.6; MyoAAV2A: 7.1; AAV9: 1.0); Liver (AVT908: 4.7; AVT913: 0.1; AVT919: 1.0; MyoAAV2A: 0.2; AAV9: 1.0).

FIG. 11. AAV-mediated GFP expression in mice. Eight-week-old BALB/c mice were intravenously injected with AAV containing CB-Fluc-2A-GFP transgene (4e11vg/mouse). AAV9 and MyoAAV 2A were used controls. Three weeks post injection, the gastrocnemius (Gas), quadriceps (Quads) and heart were collected and processed for native GFP imaging with same conditions.

FIG. 12. AAV-mediated transgene expression in the muscles of NHP. An adult male cynomolgus macaque was intravenously injected with pooled AAVs (AVT917, AVT919, AAV9 and MyoAAV 4A) at the dose of 1E13 vg/kg/capsid. Each capsid contained cyno FXN coding sequences with different tags (Flag, AU1, V5, HA) under the control of chicken beta-actin (CB) promoter and CMV enhancer. Three weeks post injection, the major muscles were collected and processed for RT-qPCR to compare the transgene mRNA level. The macaque GAPDH housekeeping gene was used as reference. The data was shown as mean with SD.

FIG. 13. AAV-mediated transgene expression in the liver of NHP. Four pooled AAV capsids containing cyno FXN coding sequences with different tags (Flag, AU1, V5, HA) under the control of chicken beta-actin (CB) promoter/CMV enhancer were intravenously injected an adult cynomolgus macaque. Three weeks post injection, the different lobes of liver were collected and processed for RT-qPCR to compare the transgene mRNA level. The tag-specific primers/probe were used for transgene transcripts. The macaque GAPDH housekeeping gene was used as reference. The data was shown as mean with SD.

FIG. 14. AAV-mediated low transgene expression in the CNS of NHP. Transgene specific RT-qPCR was used for the brain and spinal cord samples of an adult Cynomolgus macaque administrated with 4 pooled AAV capsids containing cyno FXN coding sequences with different tags (Flag, AU1, V5, HA) under the control of chicken beta-actin (CB) promoter/CMV enhancer. The macaque GAPDH housekeeping gene was used as reference for normalization. The data was shown as mean with SD.

FIG. 15. Fold change over AAV9 on the mRNA level of transgene expressed by AAV variants. An adult male cynomolgus macaque was intravenously injected with pooled 4 AAVs (AVT917, AVT919, AAV9 and MyoAAV 4A) at the dose of 1E13 vg/kg/capsid. Three weeks post injection, the major skeletal muscle tissues were harvested and processed for RT-qPCR. Transgene specific and macaque GAPDH primers/probes were used in the duplex PCR reactions. The relative transgene expression level from AAV9 was set as 1.0. The data was shown as mean with SD.

FIG. 16. Fold change over AAV9-mediated transgene mRNA level in the heart, liver and CNS of NHP. An adult male cynomolgus macaque was intravenously injected with pooled 4 AAVs (AVT917, AVT919, AAV9 and MyoAAV 4A) at the dose of 1E13 vg/kg/capsid. Three weeks post injection, the heart, liver, brain and spinal cord were harvested and processed for RT-qPCR. Transgene specific and macaque GAPDH primers/probes were used in the duplex PCR reactions. The relative transgene expression level from AAV9 was set as 1.0. The data was shown as mean with SD.

FIG. 17. Vector genome copies in the skeletal muscles of NHP after intravenous administration of AAV variants. An adult male cynomolgus macaque was intravenously injected with pooled 4 AAVs (AVT917, AVT919, AAV9 and MyoAAV 4A) at the dose of 1E13 vg/kg/capsid. Three weeks post injection, the major skeletal muscles were harvested and processed for genomic DNA extraction. Transgene specific and RNase P primers/probes were used in the ddPCR reactions. The data was shown as mean with SD.

FIG. 18. Vector genome copies in the muscles of NHP after intravenous administration of AAV variants. An adult male cynomolgus macaque was intravenously injected with pooled 4 AAVs (AVT917, AVT919, AAV9 and MyoAAV 4A) at the dose of 1E13 vg/kg/capsid. Three weeks post injection, the major muscles were harvested and processed for genomic DNA extraction. Transgene specific and RNase P primers/probes were used in the ddPCR reactions. The data was shown as mean with SD.

FIG. 19. Vector genome copies in the non-muscle tissues of NHP after intravenous administration of AAV variants. An adult male cynomolgus macaque was intravenously injected with pooled 4 AAVs (AVT917, AVT919, AAV9 and MyoAAV 4A) at the dose of 1E13 vg/kg/capsid. Three weeks post injection, the major muscles were harvested and processed for genomic DNA extraction. Transgene specific and RNase P primers/probes were used in the ddPCR reactions. The data was shown as mean with SD.

FIG. 20. Vector genome copies in the liver, pancreas, and CNS of NHP after intravenous administration of AAV variants. An adult male cynomolgus macaque was intravenously injected with pooled 4 AAVs (AVT917, AVT919, AAV9 and MyoAAV 4A) at the dose of 1E13 vg/kg/capsid. Three weeks post injection, the major muscles were harvested and processed for genomic DNA extraction. Transgene specific and RNase P primers/probes were used in the ddPCR reactions. The data was shown as mean with SD.

FIG. 21. AAV9 and variant AVT919 mediated transgene mRNA expression in NHPs. AAV9 or AVT919 containing cyno FXN coding sequences with HA tag under the control of chicken beta-actin (CB) promoter/CMV enhancer were intravenously injected to adult female cynomolgus macaques at the dose of 2E13 vg/kg. Four weeks post injection, the major tissues were harvested and processed for RT-qPCR. Transgene specific and macaque GAPDH primers/probes were used in the duplex PCR reactions. The data was shown as mean with SD.

FIG. 22. Fold change over AAV9-mediated transgene mRNA level in NHPs. AAV9 or AVT919 containing cyno FXN coding sequences with HA tag under the control of chicken beta-actin (CB) promoter/CMV enhancer were intravenously injected to adult female cynomolgus macaques at the dose of 2E13 vg/kg. Four weeks post injection, the major tissues were harvested and processed for RT-qPCR. Transgene specific and macaque GAPDH primers/probes were used in the duplex PCR reactions. The relative transgene expression level from AAV9 was set as 1.0. The data was shown as mean with SD.

FIG. 23. Immunostaining of AAV9 and variant AVT919 mediated transgene expression in NHPs. AAV9 or AVT919 containing cyno FXN coding sequences with HA tag under the control of chicken beta-actin (CB) promoter/CMV enhancer were intravenously injected to adult female cynomolgus macaques at dose of 2E13 vg/kg. Four weeks post injection, the major tissues were harvested and processed for immunostaining. The rabbit anti-HA monoclonal antibody was used as primary antibody. The anti-rabbit HRP-conjugated antibody and DAB substrate were used for detection.

### DETAILED DESCRIPTION

The present disclosure provides variant adeno-associated virus serotype 9 (AAV9) capsid proteins and recombinant AAV9 particles comprising the variant AAV9 capsid proteins. The present disclosure describes that various variant AAV9 capsid proteins and recombinant AAV9 particles comprising these variant AAV9 capsid proteins have an increased tropism for certain tissue (s) relative to the wild-type counterpart. For example, some variant AAV9 capsid proteins and recombinant AAV9 particles described herein have an increased tropism for muscle relative to their wild-type counterpart. Some variant AAV9 capsid proteins and recombinant AAV9 particles described herein have an increased tropism for liver relative to their wild-type counterpart. Some variant AAV9 capsid proteins and recombinant AAV9 particles described herein have an increased tropism for brain relative to their wild-type counterpart.

The present disclosure also provides pharmaceutical compositions comprising the recombinant AAV9 particles, polynucleotides encoding the variant AAV9 capsid proteins, vectors and host cells comprising the polynucleotides, populations of cells transduced by the recombinant AAV9 particles, as well as various methods using the recombinant AAV9 particles.

### Definitions

As used in this disclosure, the singular forms “a”, “an” and “the” include plural referents unless the context clearly dictates otherwise. The terms “a” (or “an”), as well as the terms “one or more,” and “at least one” can be used interchangeably herein unless the context clearly dictates otherwise.

The terms “or” and “and” can be used interchangeably and can be understood to mean “and/or” unless the context clearly dictates otherwise.

As used in this disclosure and unless otherwise specified, the terms “about” and “approximately” shall be construed so as to allow normal variation as judged by a person of skill in the art, such as, for example, a variation within 20% or 10% or 5% of the stated value. In specific embodiments, the terms “about” and “approximately” encompass the exact value recited. Unless the context clearly dictates otherwise, all numerical values provided herein are modified by the term about.

It is understood that wherever aspects and embodiments are described in this disclosure with the language “comprising,” otherwise analogous aspects described in terms of “consisting of” and/or “consisting essentially of” are also provided.

As used herein, the term “therapeutically effective amount” refers to an amount of a biologic molecule which is sufficient to reduce and/or ameliorate the severity and/or duration of a given disease or disorder and/or a symptom related thereto. This term also encompasses an amount necessary for the reduction, slowing, or amelioration of the advancement or progression of a given disease or disorder, reduction, slowing, or amelioration of the recurrence, development or onset of a given disease or disorder, and/or to improve or enhance the prophylactic or therapeutic effect (s) of another therapy.

As used herein, the terms “subject” and “patient” are used interchangeably. As used herein, a subject is a mammal such as a non-primate (e.g., cows, pigs, horses, cats, dogs, goats, rabbits, rats, mice, etc.) or a primate (e.g., monkey and human), for example a human. In one embodiment, the subject is a mammal, e.g., a human, diagnosed with a disease or disorder. In another embodiment, the subject is a mammal, e.g., a human, at risk of developing a disease or disorder. In another embodiment, the subject is a non-human primate. In specific embodiments, the subject is a human. In a specific embodiment, the subject is a human adult. In a specific embodiment, the subject is a human adolescent. In a specific embodiment, the subject is a human child.

### Novel AAV9 Capsid Proteins

In one aspect, provided herein is a variant adeno-associated virus serotype 9 (AAV9) capsid protein comprising the amino acid sequence of SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, or SEQ ID NO: 168.

A variant AAV9 capsid protein is an AAV9 capsid protein having an amino acid sequence that is different from the amino acid sequence of wild-type AAV9 VP1 capsid protein, which is set forth in SEQ ID NO: 2, and different from the amino acid sequences of the VP2 and VP3 counterparts of AAV9 VP1 whose amino acid sequence is set forth in SEQ ID NO: 2 (the amino acid sequences of the AAV9 VP2 and VP3 counterparts are set forth in SEQ ID NOs: 178 and 179, respectively). In preferred embodiments, a variant AAV9 capsid protein described herein is a non-naturally occurring AAV9 capsid protein.

In certain embodiments, a variant AAV9 capsid protein provided herein comprises the amino acid sequence of SEQ ID NO: 37. In specific embodiments, a variant AAV9 capsid protein provided herein comprises the amino acid sequence of SEQ ID NO: 6.

In certain embodiments, a variant AAV9 capsid protein provided herein comprises the amino acid sequence of SEQ ID NO: 39. In specific embodiments, a variant AAV9 capsid protein provided herein comprises the amino acid sequence of SEQ ID NO: 7.

In certain embodiments, a variant AAV9 capsid protein provided herein comprises the amino acid sequence of SEQ ID NO: 41. In specific embodiments, a variant AAV9 capsid protein provided herein comprises the amino acid sequence of SEQ ID NO: 8.

In certain embodiments, a variant AAV9 capsid protein provided herein comprises the amino acid sequence of SEQ ID NO: 43. In specific embodiments, a variant AAV9 capsid protein provided herein comprises the amino acid sequence of SEQ ID NO: 9.

In certain embodiments, a variant AAV9 capsid protein provided herein comprises the amino acid sequence of SEQ ID NO: 45. In specific embodiments, a variant AAV9 capsid protein provided herein comprises the amino acid sequence of SEQ ID NO: 10.

In certain embodiments, a variant AAV9 capsid protein provided herein comprises the amino acid sequence of SEQ ID NO: 47. In certain embodiments, a variant AAV9 capsid protein provided herein comprises the amino acid sequence of SEQ ID NO: 49. In certain embodiments, a variant AAV9 capsid protein provided herein comprises the amino acid sequence of SEQ ID NO: 47 and comprises the amino acid sequence of SEQ ID NO: 49. In specific embodiments, a variant AAV9 capsid protein provided herein comprises the amino acid sequence of SEQ ID NO: 11.

In certain embodiments, a variant AAV9 capsid protein provided herein comprises the amino acid sequence of SEQ ID NO: 51. In specific embodiments, a variant AAV9 capsid protein provided herein comprises the amino acid sequence of SEQ ID NO: 12.

In certain embodiments, a variant AAV9 capsid protein provided herein comprises the amino acid sequence of SEQ ID NO: 53. In specific embodiments, a variant AAV9 capsid protein provided herein comprises the amino acid sequence of SEQ ID NO: 13.

In certain embodiments, a variant AAV9 capsid protein provided herein comprises the amino acid sequence of SEQ ID NO: 55. In specific embodiments, a variant AAV9 capsid protein provided herein comprises the amino acid sequence of SEQ ID NO: 14.

In certain embodiments, a variant AAV9 capsid protein provided herein comprises the amino acid sequence of SEQ ID NO: 57. In specific embodiments, a variant AAV9 capsid protein provided herein comprises the amino acid sequence of SEQ ID NO: 15.

In certain embodiments, a variant AAV9 capsid protein provided herein comprises the amino acid sequence of SEQ ID NO: 59. In certain embodiments, a variant AAV9 capsid protein provided herein comprises the amino acid sequence of SEQ ID NO: 61. In certain embodiments, a variant AAV9 capsid protein provided herein comprises the amino acid sequence of SEQ ID NO: 59 and comprises the amino acid sequence of SEQ ID NO: 61. In specific embodiments, a variant AAV9 capsid protein provided herein comprises the amino acid sequence of SEQ ID NO: 16.

In certain embodiments, a variant AAV9 capsid protein provided herein comprises the amino acid sequence of SEQ ID NO: 63. In specific embodiments, a variant AAV9 capsid protein provided herein comprises the amino acid sequence of SEQ ID NO: 17.

In certain embodiments, a variant AAV9 capsid protein provided herein comprises the amino acid sequence of SEQ ID NO: 65. In specific embodiments, a variant AAV9 capsid protein provided herein comprises the amino acid sequence of SEQ ID NO: 18.

In certain embodiments, a variant AAV9 capsid protein provided herein comprises the amino acid sequence of SEQ ID NO: 67. In specific embodiments, a variant AAV9 capsid protein provided herein comprises the amino acid sequence of SEQ ID NO: 19.

In certain embodiments, a variant AAV9 capsid protein provided herein comprises the amino acid sequence of SEQ ID NO: 69. In specific embodiments, a variant AAV9 capsid protein provided herein comprises the amino acid sequence of SEQ ID NO: 20.

In certain embodiments, a variant AAV9 capsid protein provided herein comprises the amino acid sequence of SEQ ID NO: 71. In specific embodiments, a variant AAV9 capsid protein provided herein comprises the amino acid sequence of SEQ ID NO: 21.

In certain embodiments, a variant AAV9 capsid protein provided herein comprises the amino acid sequence of SEQ ID NO: 73. In specific embodiments, a variant AAV9 capsid protein provided herein comprises the amino acid sequence of SEQ ID NO: 22.

In certain embodiments, a variant AAV9 capsid protein provided herein comprises the amino acid sequence of SEQ ID NO: 75. In specific embodiments, a variant AAV9 capsid protein provided herein comprises the amino acid sequence of SEQ ID NO: 23.

In certain embodiments, a variant AAV9 capsid protein provided herein comprises the amino acid sequence of SEQ ID NO: 77. In specific embodiments, a variant AAV9 capsid protein provided herein comprises the amino acid sequence of SEQ ID NO: 24.

In certain embodiments, a variant AAV9 capsid protein provided herein comprises the amino acid sequence of SEQ ID NO: 79. In specific embodiments, a variant AAV9 capsid protein provided herein comprises the amino acid sequence of SEQ ID NO: 25.

In certain embodiments, a variant AAV9 capsid protein provided herein comprises the amino acid sequence of SEQ ID NO: 80. In specific embodiments, a variant AAV9 capsid protein provided herein comprises the amino acid sequence of SEQ ID NO: 26.

In certain embodiments, a variant AAV9 capsid protein provided herein comprises the amino acid sequence of SEQ ID NO: 81. In specific embodiments, a variant AAV9 capsid protein provided herein comprises the amino acid sequence of SEQ ID NO: 27.

In certain embodiments, a variant AAV9 capsid protein provided herein comprises the amino acid sequence of SEQ ID NO: 82. In specific embodiments, a variant AAV9 capsid protein provided herein comprises the amino acid sequence of SEQ ID NO: 28.

In certain embodiments, a variant AAV9 capsid protein provided herein comprises the amino acid sequence of SEQ ID NO: 83. In specific embodiments, a variant AAV9 capsid protein provided herein comprises the amino acid sequence of SEQ ID NO: 29.

In certain embodiments, a variant AAV9 capsid protein provided herein comprises the amino acid sequence of SEQ ID NO: 84. In specific embodiments, a variant AAV9 capsid protein provided herein comprises the amino acid sequence of SEQ ID NO: 30.

In certain embodiments, a variant AAV9 capsid protein provided herein comprises the amino acid sequence of SEQ ID NO: 85. In specific embodiments, a variant AAV9 capsid protein provided herein comprises the amino acid sequence of SEQ ID NO: 31.

In certain embodiments, a variant AAV9 capsid protein provided herein comprises the amino acid sequence of SEQ ID NO: 86. In specific embodiments, a variant AAV9 capsid protein provided herein comprises the amino acid sequence of SEQ ID NO: 32.

In certain embodiments, a variant AAV9 capsid protein provided herein comprises the amino acid sequence of SEQ ID NO: 87. In specific embodiments, a variant AAV9 capsid protein provided herein comprises the amino acid sequence of SEQ ID NO: 33.

In certain embodiments, a variant AAV9 capsid protein provided herein comprises the amino acid sequence of SEQ ID NO: 116. In specific embodiments, a variant AAV9 capsid protein provided herein comprises the amino acid sequence of SEQ ID NO: 115.

In certain embodiments, a variant AAV9 capsid protein provided herein comprises the amino acid sequence of SEQ ID NO: 118. In specific embodiments, a variant AAV9 capsid protein provided herein comprises the amino acid sequence of SEQ ID NO: 117.

In certain embodiments, a variant AAV9 capsid protein provided herein comprises the amino acid sequence of SEQ ID NO: 120. In specific embodiments, a variant AAV9 capsid protein provided herein comprises the amino acid sequence of SEQ ID NO: 119.

In certain embodiments, a variant AAV9 capsid protein provided herein comprises the amino acid sequence of SEQ ID NO: 122. In specific embodiments, a variant AAV9 capsid protein provided herein comprises the amino acid sequence of SEQ ID NO: 121.

In certain embodiments, a variant AAV9 capsid protein provided herein comprises the amino acid sequence of SEQ ID NO: 124. In specific embodiments, a variant AAV9 capsid protein provided herein comprises the amino acid sequence of SEQ ID NO: 123.

In certain embodiments, a variant AAV9 capsid protein provided herein comprises the amino acid sequence of SEQ ID NO: 126. In specific embodiments, a variant AAV9 capsid protein provided herein comprises the amino acid sequence of SEQ ID NO: 125.

In certain embodiments, a variant AAV9 capsid protein provided herein comprises the amino acid sequence of SEQ ID NO: 128. In specific embodiments, a variant AAV9 capsid protein provided herein comprises the amino acid sequence of SEQ ID NO: 127.

In certain embodiments, a variant AAV9 capsid protein provided herein comprises the amino acid sequence of SEQ ID NO: 130. In specific embodiments, a variant AAV9 capsid protein provided herein comprises the amino acid sequence of SEQ ID NO: 129.

In certain embodiments, a variant AAV9 capsid protein provided herein comprises the amino acid sequence of SEQ ID NO: 132. In specific embodiments, a variant AAV9 capsid protein provided herein comprises the amino acid sequence of SEQ ID NO: 131.

In certain embodiments, a variant AAV9 capsid protein provided herein comprises the amino acid sequence of SEQ ID NO: 134. In specific embodiments, a variant AAV9 capsid protein provided herein comprises the amino acid sequence of SEQ ID NO: 133.

In certain embodiments, a variant AAV9 capsid protein provided herein comprises the amino acid sequence of SEQ ID NO: 136. In specific embodiments, a variant AAV9 capsid protein provided herein comprises the amino acid sequence of SEQ ID NO: 135.

In certain embodiments, a variant AAV9 capsid protein provided herein comprises the amino acid sequence of SEQ ID NO: 138. In specific embodiments, a variant AAV9 capsid protein provided herein comprises the amino acid sequence of SEQ ID NO: 137.

In certain embodiments, a variant AAV9 capsid protein provided herein comprises the amino acid sequence of SEQ ID NO: 140. In specific embodiments, a variant AAV9 capsid protein provided herein comprises the amino acid sequence of SEQ ID NO: 139.

In certain embodiments, a variant AAV9 capsid protein provided herein comprises the amino acid sequence of SEQ ID NO: 142. In specific embodiments, a variant AAV9 capsid protein provided herein comprises the amino acid sequence of SEQ ID NO: 141.

In certain embodiments, a variant AAV9 capsid protein provided herein comprises the amino acid sequence of SEQ ID NO: 144. In specific embodiments, a variant AAV9 capsid protein provided herein comprises the amino acid sequence of SEQ ID NO: 143.

In certain embodiments, a variant AAV9 capsid protein provided herein comprises the amino acid sequence of SEQ ID NO: 146. In specific embodiments, a variant AAV9 capsid protein provided herein comprises the amino acid sequence of SEQ ID NO: 145.

In certain embodiments, a variant AAV9 capsid protein provided herein comprises the amino acid sequence of SEQ ID NO: 148. In specific embodiments, a variant AAV9 capsid protein provided herein comprises the amino acid sequence of SEQ ID NO: 147.

In certain embodiments, a variant AAV9 capsid protein provided herein comprises the amino acid sequence of SEQ ID NO: 150. In specific embodiments, a variant AAV9 capsid protein provided herein comprises the amino acid sequence of SEQ ID NO: 149.

In certain embodiments, a variant AAV9 capsid protein provided herein comprises the amino acid sequence of SEQ ID NO: 152. In specific embodiments, a variant AAV9 capsid protein provided herein comprises the amino acid sequence of SEQ ID NO: 151.

In certain embodiments, a variant AAV9 capsid protein provided herein comprises the amino acid sequence of SEQ ID NO: 154. In specific embodiments, a variant AAV9 capsid protein provided herein comprises the amino acid sequence of SEQ ID NO: 153.

In certain embodiments, a variant AAV9 capsid protein provided herein comprises the amino acid sequence of SEQ ID NO: 156. In specific embodiments, a variant AAV9 capsid protein provided herein comprises the amino acid sequence of SEQ ID NO: 155.

In certain embodiments, a variant AAV9 capsid protein provided herein comprises the amino acid sequence of SEQ ID NO: 158. In specific embodiments, a variant AAV9 capsid protein provided herein comprises the amino acid sequence of SEQ ID NO: 157.

In certain embodiments, a variant AAV9 capsid protein provided herein comprises the amino acid sequence of SEQ ID NO: 160. In specific embodiments, a variant AAV9 capsid protein provided herein comprises the amino acid sequence of SEQ ID NO: 159.

In certain embodiments, a variant AAV9 capsid protein provided herein comprises the amino acid sequence of SEQ ID NO: 162. In specific embodiments, a variant AAV9 capsid protein provided herein comprises the amino acid sequence of SEQ ID NO: 161.

In certain embodiments, a variant AAV9 capsid protein provided herein comprises the amino acid sequence of SEQ ID NO: 164. In specific embodiments, a variant AAV9 capsid protein provided herein comprises the amino acid sequence of SEQ ID NO: 163.

In certain embodiments, a variant AAV9 capsid protein provided herein comprises the amino acid sequence of SEQ ID NO: 166. In specific embodiments, a variant AAV9 capsid protein provided herein comprises the amino acid sequence of SEQ ID NO: 165.

In certain embodiments, a variant AAV9 capsid protein provided herein comprises the amino acid sequence of SEQ ID NO: 168. In specific embodiments, a variant AAV9 capsid protein provided herein comprises the amino acid sequence of SEQ ID NO: 167.

An AAV capsid typically consists of three viral proteins, VP1, VP2 and VP3, which are alternative splice variants. Often, the three proteins are encoded by the same nucleotide sequence, with the VP1 amino acid sequence encompassing the entire amino acid sequence of VP2, and the VP2 amino acid sequence encompassing the entire amino acid sequence of VP3.

Typically, AAV capsid proteins comprise a number of variable regions (VRs) and constant regions, which are located between the variable regions. The “GH loop” refers to a loop sequence flanked by β-strand G and β-strand H within the internal β-barrel of an AAV capsid protein, and comprises variable region VR IV through VR VIII.

The sequences of the VP2 and VP3 capsid proteins and the locations of various domains such as the variable regions and the GH loop can be readily and routinely determined by a skilled person from a given VP1 sequence by a suitable method known in the art, for example, by comparing or aligning the VP1 sequence with an annotated VP1 sequence (preferably, an annotated VP1 sequence from a closely related AAV species) using a suitable algorithm. Preferably, such an annotated VP1 sequence is an annotated wild-type AAV VP1 sequence (e.g., an annotated wild-type AAV9 VP1 sequence, such as the amino acid sequence set forth in SEQ ID NO: 2).

In various embodiments, a variant AAV9 capsid protein described herein is a variant AAV9 VP1 capsid protein. Also provided herein are variant AAV9 capsid proteins that are the VP2 and VP3 counterparts of the VP1 capsid proteins described herein. Further provided herein are fragments of such variant AAV9 capsid proteins (e.g., fragments of VP1, VP2 or VP3 capsid proteins), including, without limitation, variable regions (e.g., VR IV and VR VIII), GH loop and functional fragments that essentially retain the biological activity of the corresponding capsid proteins and their associated tissue tropism. In certain embodiments, fragments of a VP1, VP2 or VP3 capsid protein described herein are at least 7 amino acids in length. In certain embodiments, fragments of a VP1, VP2 or VP3 capsid protein described herein are at least 8 amino acids in length. In certain embodiments, fragments of a VP1, VP2 or VP3 capsid protein described herein are at least 9 amino acids in length. In certain embodiments, fragments of a VP1, VP2 or VP3 capsid protein described herein are at least 10 amino acids in length. In certain embodiments, fragments of a VP1, VP2 or VP3 capsid protein described herein are at least 20 amino acids in length. In certain embodiments, fragments of a VP1, VP2 or VP3 capsid protein described herein are at least 30 amino acids in length. In certain embodiments, fragments of a VP1, VP2 or VP3 capsid protein described herein are at least 40 amino acids in length. In certain embodiments, fragments of a VP1, VP2 or VP3 capsid protein described herein are at least 50 amino acids in length. In certain embodiments, fragments of a VP1, VP2 or VP3 capsid protein described herein are at least 100 amino acids in length. In certain embodiments, fragments of a VP1, VP2 or VP3 capsid protein described herein are at least 200 amino acids in length. In certain embodiments, fragments of a VP1, VP2 or VP3 capsid protein described herein are at least 300 amino acids in length. In certain embodiments, fragments of a VP1, VP2 or VP3 capsid protein described herein are at least 400 amino acids in length. In certain embodiments, fragments of a VP1, VP2 or VP3 capsid protein described herein are at least 500 amino acids in length. In certain embodiments, fragments of a VP1, VP2 or VP3 capsid protein described herein are 7-10 amino acids in length. In certain embodiments, fragments of a VP1, VP2 or VP3 capsid protein described herein are 10-50 amino acids in length. In certain embodiments, fragments of a VP1, VP2 or VP3 capsid protein described herein are 50-100 amino acids in length. In certain embodiments, fragments of a VP1, VP2 or VP3 capsid protein described herein are 100-200 amino acids in length. In certain embodiments, fragments of a VP1, VP2 or VP3 capsid protein described herein are 200-300 amino acids in length. In certain embodiments, fragments of a VP1, VP2 or VP3 capsid protein described herein are 300-400 amino acids in length. In certain embodiments, fragments of a VP1, VP2 or VP3 capsid protein described herein are 400-500 amino acids in length.

In various embodiments, a variant AAV9 capsid protein described herein is associated with an increased tropism for one or more tissues relative to a wild-type AAV9 capsid protein (preferably, a wild-type AAV9 capsid protein comprising the amino acid sequence of SEQ ID NO: 2). A variant AAV9 capsid protein being associated with an increased tropism for a tissue shall be construed to mean that a recombinant AAV9 particle comprising the variant AAV9 capsid protein has an increased tropism for that tissue.

In certain embodiments, a variant AAV9 capsid protein described herein is associated with an increased tropism for muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for skeletal muscle (e.g., biceps, triceps, quadriceps, tibialis anterior, gastrocnemius, rectus abdominis, diaphragm and/or pectoralis major) relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for heart muscle (e.g., heart atrium muscle and/or heart ventricle muscle) relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for biceps, triceps, quadriceps, tibialis anterior, gastrocnemius, rectus abdominis, diaphragm, pectoralis major, heart atrium muscle and/or heart ventricle muscle relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for biceps, triceps, quadriceps, tibialis anterior, gastrocnemius, rectus abdominis, diaphragm and/or pectoralis major relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for biceps relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for triceps relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for quadriceps relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for tibialis anterior relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for gastrocnemius relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for rectus abdominis relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for diaphragm relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for pectoralis major relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for heart atrium muscle relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for heart ventricle muscle relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for liver relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for brain (e.g., middle front brain, middle rear brain, front brain and/or rear brain) relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for middle front brain relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for middle rear brain relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for front brain relative to a wild-type AAV9 capsid protein (preferably, a wild-type AAV9 capsid protein comprising the amino acid sequence of SEQ ID NO: 2). In a specific embodiment, a variant AAV9 capsid protein described herein is associated with an increased tropism for rear brain relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for both muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and liver relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for both muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and brain (e.g., middle front brain, middle rear brain, front brain and/or rear brain) relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for both liver and brain (e.g., middle front brain, middle rear brain, front brain and/or rear brain) relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for kidney relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for lung relative to a wild-type AAV9 capsid protein (preferably, a wild-type AAV9 capsid protein comprising the amino acid sequence of SEQ ID NO: 2).

In certain embodiments, a variant AAV9 capsid protein described herein is associated with an increased tropism for spleen relative to a wild-type AAV9 capsid protein (preferably, a wild-type AAV9 capsid protein comprising the amino acid sequence of SEQ ID NO: 2).

In various embodiments, a variant AAV9 capsid protein described herein is associated with an increased tropism for one or more tissues relative to the capsid protein of MyoAAV 1A (preferably, the MyoAAV 1A capsid protein comprising the amino acid sequence of SEQ ID NO: 3), the capsid protein of MyoAAV 2A (preferably, the MyoAAV 2A capsid protein comprising the amino acid sequence of SEQ ID NO: 4), the capsid protein of MyoAAV 3A (preferably, the MyoAAV 3A capsid protein comprising the amino acid sequence of SEQ ID NO: 111), and/or the capsid protein of MyoAAV 4A (preferably, the MyoAAV 4A capsid protein comprising the amino acid sequence of SEQ ID NO: 5). In certain embodiments, the one or more tissues are or comprise muscle (e.g., skeletal muscle and/or heart muscle) In specific embodiments, the one or more tissues are or comprise skeletal muscle (e.g., biceps, triceps, quadriceps, tibialis anterior, gastrocnemius, rectus abdominis, diaphragm and/or pectoralis major). In specific embodiments, the one or more tissues are or comprise heart muscle (e.g., heart atrium muscle and/or heart ventricle muscle) In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and liver. In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and brain (e.g., middle front brain, middle rear brain, front brain and/or rear brain). In certain embodiments, the one or more tissues are or comprise liver and brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a variant AAV9 capsid protein described herein is associated with a decreased tropism for one or more tissues relative to a wild-type AAV9 capsid protein (preferably, a wild-type AAV9 capsid protein comprising the amino acid sequence of SEQ ID NO: 2). A variant AAV9 capsid protein being associated with a decreased tropism for a tissue shall be construed to mean that a recombinant AAV9 particle comprising the variant AAV9 capsid protein has a decreased tropism for that tissue. In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)). In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a variant AAV9 capsid protein described herein is associated with a decreased tropism for one or more tissues relative to the capsid protein of MyoAAV 1A (preferably, the MyoAAV 1A capsid protein comprising the amino acid sequence of SEQ ID NO: 3), the capsid protein of MyoAAV 2A (preferably, the MyoAAV 2A capsid protein comprising the amino acid sequence of SEQ ID NO: 4), the capsid protein of MyoAAV 3A (preferably, the MyoAAV 3A capsid protein comprising the amino acid sequence of SEQ ID NO: 111), and/or the capsid protein of MyoAAV 4A (preferably, the MyoAAV 4A capsid protein comprising the amino acid sequence of SEQ ID NO: 5). In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)). In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a variant AAV9 capsid protein described herein is associated with an increased tropism for one or more tissues as described above and a decreased tropism for another one or more tissues as described above.

In various embodiments, the tropism for a tissue described herein is measured after a subject is administered systemically with the corresponding AAV9 particle. In certain embodiments, the tropism for a tissue described herein is measured after a subject is administered intravenously with the corresponding AAV9 particle.

In certain embodiments, a variant AAV9 capsid protein described herein is isolated. In certain embodiments, a variant AAV9 capsid protein described herein is purified.

### Recombinant AAV9 Particles

In another aspect, provided herein is a recombinant AAV9 particle comprising a variant AAV9 capsid protein described herein (e.g., a variant AAV9 capsid protein described in Section 5.2). Also provided herein are recombinant AAV9 particles comprising a fragment of a variant AAV9 capsid protein described herein (e.g., a fragment as described in Section 5.2).

An “AAV particle” refers to an AAV virus composed of at least one AAV capsid protein and an encapsidated AAV genome. An AAV9 particle is therefore an AAV serotype 9 virus.

The AAV genome is a linear, single-stranded DNA molecule that contains inverted terminal repeats (ITRs) at the 5′ and 3′ termini of the viral genome. The ITRs function in cis as origins of DNA replication and as packaging signals for the viral genome.

In various embodiments, a recombinant AAV9 particle described herein comprises a variant AAV9 capsid protein described herein and a recombinant AAV genome comprising a heterologous nucleotide sequence flanked by AAV ITRs (e.g., AAV9 ITRs or AAV2 ITRs), wherein the heterologous nucleotide sequence is heterologous to the AAV ITRs. In preferred embodiments, the recombinant AAV genome does not comprise a functional AAV cap gene. In preferred embodiments, the recombinant AAV genome does not comprise a functional AAV rep gene. In preferred embodiments, the recombinant AAV genome does not comprise a functional AAV cap gene and does not comprise a functional AAV rep gene.

In certain embodiments, the heterologous nucleotide sequence comprises a nucleotide sequence encoding a biologic molecule. The biologic molecule can be, for example, but not limited to, a polypeptide, a protein, a nucleic acid (e.g., DNA or RNA), or an oligonucleotide (e.g., siRNA, shRNA, miRNA or aptamer). In specific embodiments, the biologic molecule is a polypeptide or a protein. In preferred embodiments, the biologic molecule is a human polypeptide or a human protein. The biologic molecule can be a reporter molecule, such as a reporter protein (for example, a fluorescent protein (e.g., green fluorescent protein (GFP)), luciferase (e.g., firefly luciferase), β-lactamase, or β-galactosidase (LacZ)). The biologic molecule can also be a therapeutic molecule, such as a therapeutic protein. A therapeutic molecule may be used to correct or ameliorate gene deficiencies associated with a disease or disorder. Exemplary therapeutic molecules may include, without limitation, enzymes, cytokines, growth factors, kinases, dominant negative mutant proteins, antibodies and antigen-binding fragments thereof, and interleukins. In specific embodiments, the biologic molecule is expressed in muscle cells (e.g., expressed at a higher level in muscle cells relative to other cell types). In specific embodiments, the biologic molecule is expressed in liver cells (e.g., expressed at a higher level in liver cells relative to other cell types). In specific embodiments, the biologic molecule is expressed in brain cells (e.g., expressed at a higher level in brain cells relative to other cell types). In specific embodiments, the biologic molecule is expressed in both muscle cells and liver cells (e.g., expressed at higher level in both muscle cells and liver cells relative to other cell types). In specific embodiments, the biologic molecule is expressed in both muscle cells and brain cells (e.g., expressed at higher level in both muscle cells and brain cells relative to other cell types). In specific embodiments, the biologic molecule is expressed in both liver cells and brain cells (e.g., expressed at higher level in both liver cells and brain cells relative to other cell types). In specific embodiments, the biologic molecule is expressed in kidney cells (e.g., expressed at a higher level in kidney cells relative to other cell types). In specific embodiments, the biologic molecule is expressed in lung cells (e.g., expressed at a higher level in lung cells relative to other cell types). In specific embodiments, the biologic molecule is expressed in spleen cells (e.g., expressed at a higher level in spleen cells relative to other cell types). In specific embodiments, the biologic molecule functions in muscle cells (e.g., is required or important for the normal function of muscle cells). In specific embodiments, the biologic molecule functions in liver cells (e.g., is required or important for the normal function of liver cells). In specific embodiments, the biologic molecule functions in brain cells (e.g., is required or important for the normal function of brain cells). In specific embodiments, the biologic molecule functions in both muscle cells and liver cells (e.g., is required or important for the normal function of both muscle cells and liver cells). In specific embodiments, the biologic molecule functions in both muscle cells and brain cells (e.g., is required or important for the normal function of both muscle cells and brain cells). In specific embodiments, the biologic molecule functions in both liver cells and brain cells (e.g., is required or important for the normal function of both liver cells and brain cells). In specific embodiments, the biologic molecule functions in kidney cells (e.g., is required or important for the normal function of kidney cells). In specific embodiments, the biologic molecule functions in lung cells (e.g., is required or important for the normal function of lung cells). In specific embodiments, the biologic molecule functions in spleen cells (e.g., is required or important for the normal function of spleen cells). In specific embodiments, the muscle cells described herein are skeletal muscle cells (e.g., biceps, triceps, quadriceps, tibialis anterior, gastrocnemius, rectus abdominis, diaphragm and/or pectoralis major cells) and/or heart muscle cells (e.g., heart atrium muscle and/or heart ventricle muscle cells). In specific embodiments, the brain cells described herein are middle front brain, middle rear brain, front brain and/or rear brain cells. In a specific embodiment, the biologic molecule is a functional Survival Motor Neuron (SMN) protein (e.g., a wild-type SMN protein). In a specific embodiment, the biologic molecule is a functional microdystrophin (e.g., a wild-type microdystrophin). In a specific embodiment, the biologic molecule is a functional alpha-galactosidase (e.g., a wild-type alpha-galactosidase). In a specific embodiment, the biologic molecule is a functional phenylalanine hydroxylase (PAH) (e.g., a wild-type PAH). In a specific embodiment, the biologic molecule is a functional Coagulation Factor VIII (FVIII) (e.g., a wild-type FVIII). In a specific embodiment, the biologic molecule is a functional Coagulation Factor IX (FIX) (e.g., a wild-type FIX). In a specific embodiment, the biologic molecule is a functional beta-glucocerebrosidase (GBA) (e.g., a wild-type GBA). In a specific embodiment, the biologic molecule is NPC Intracellular Cholesterol Transporter 1 (NPC1) (e.g., a wild-type NPC1). In a specific embodiment, the biologic molecule is NPC Intracellular Cholesterol Transporter 2 (NPC2) (e.g., a wild-type NPC2). In a specific embodiment, the biologic molecule is acid alpha-glucosidase (GAA) (e.g., a wild-type GAA).

In certain embodiments, the heterologous nucleotide sequence further comprises one or more nucleotide sequences encoding one or more regulatory control elements, wherein the one or more nucleotide sequences encoding the one or more regulatory control elements are operably linked to the nucleotide sequence encoding the biologic molecule. The phrase “operably linked” shall be construed in this context to mean that the nucleotide sequences are linked in a manner that permits transcription, translation, and/or expression of the biologic molecule in a cell transduced by the AAV virus that comprises a recombinant AAV genome containing the heterologous nucleotide sequence.

Regulatory control elements include both expression control elements that are contiguous with the biologic molecule-encoding nucleotide sequence of interest and expression control elements that act in trans or at a distance to control the expression of the biologic molecule of interest.

Expression control elements include, without limitation, appropriate transcription initiation, termination, promoter and enhancer sequences; polyadenylation (polyA) signals; sequences that stabilize cytoplasmic mRNA; sequences that enhance translation efficiency (i.e., Kozak consensus sequence); sequences that enhance protein stability; secretion signals; and nuclear localization sequences.

In various embodiments, the regulatory control element is a promoter. In certain embodiments, the promoter is a constitutive promoter. In certain embodiments, the promoter is an inducible promoter. In certain embodiments, the promoter is a native promoter of the biologic molecule-encoding nucleotide sequence. In certain embodiments, the promoter is a tissue-specific promoter. In preferred embodiments, the tissue-specific promoter is a muscle-specific promoter. In a specific embodiment, the tissue-specific promoter is a skeletal muscle (e.g., biceps, triceps, quadriceps, tibialis anterior, gastrocnemius, rectus abdominis, diaphragm and/or pectoralis major)-specific promoter. In a specific embodiment, the tissue-specific promoter is a heart muscle (e.g., heart atrium muscle and/or heart ventricle muscle)-specific promoter. In specific embodiments, the tissue-specific promoter is a liver-specific promoter. In specific embodiments, the tissue-specific promoter is a brain (e.g., middle front brain, middle rear brain, front brain and/or rear brain)-specific promoter. In specific embodiments, the tissue-specific promoter is a kidney-specific promoter. In specific embodiments, the tissue-specific promoter is a lung-specific promoter. In specific embodiments, the tissue-specific promoter is a spleen-specific promoter. Non-limiting exemplary muscle-specific promoters include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17: 241-245 (1999)).

In various embodiments, the heterologous nucleotide sequence comprises an expression cassette, which comprises a nucleotide sequence encoding a biologic molecule as described herein and one or more nucleotide sequences encoding one or more regulatory control elements as described herein (e.g., a nucleotide sequence encoding a promoter and a nucleotide sequence encoding a polyA signal), wherein the one or more nucleotide sequences encoding the one or more regulatory control elements are operably linked to the nucleotide sequence encoding the biologic molecule.

In various embodiments, a recombinant AAV9 particle described herein has an increased tropism for one or more tissues relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for skeletal muscle (e.g., biceps, triceps, quadriceps, tibialis anterior, gastrocnemius, rectus abdominis, diaphragm and/or pectoralis major) relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for heart muscle (e.g., heart atrium muscle and/or heart ventricle muscle) relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for biceps, triceps, quadriceps, tibialis anterior, gastrocnemius, rectus abdominis, diaphragm, pectoralis major, heart atrium muscle and/or heart ventricle muscle relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for biceps, triceps, quadriceps, tibialis anterior, gastrocnemius, rectus abdominis, diaphragm and/or pectoralis major relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for biceps relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for triceps relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for quadriceps relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for tibialis anterior relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for gastrocnemius relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for rectus abdominis relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for diaphragm relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for pectoralis major relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for heart atrium muscle relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for heart ventricle muscle relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for liver relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for brain (e.g., middle front brain, middle rear brain, front brain and/or rear brain) relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for middle front brain relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for middle rear brain relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for front brain relative to a wild-type AAV9 particle (preferably, a wild-type AAV9 particle having a capsid protein comprising the amino acid sequence of SEQ ID NO: 2). In a specific embodiment, a recombinant AAV9 particle described herein has an increased tropism for rear brain relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for both muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and liver relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for both muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and brain (e.g., middle front brain, middle rear brain, front brain and/or rear brain) relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for both liver and brain (e.g., middle front brain, middle rear brain, front brain and/or rear brain) relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for kidney relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for lung relative to a wild-type AAV9 particle (preferably, a wild-type AAV9 particle having a capsid protein comprising the amino acid sequence of SEQ ID NO: 2).

In certain embodiments, a recombinant AAV9 particle described herein has an increased tropism for spleen relative to a wild-type AAV9 particle (preferably, a wild-type AAV9 particle having a capsid protein comprising the amino acid sequence of SEQ ID NO: 2).

In various embodiments, a recombinant AAV9 particle described herein has an increased tropism for one or more tissues relative to MyoAAV 1A (preferably, MyoAAV 1A having a capsid protein comprising the amino acid sequence of SEQ ID NO: 3), MyoAAV 2A (preferably, MyoAAV 2A having a capsid protein comprising the amino acid sequence of SEQ ID NO: 4), MyoAAV 3A (preferably, MyoAAV 3A having a capsid protein comprising the amino acid sequence of SEQ ID NO: 111), and/or MyoAAV 4A (preferably, MyoAAV 4A having a capsid protein comprising the amino acid sequence of SEQ ID NO: 5). In certain embodiments, the one or more tissues are or comprise muscle (e.g., skeletal muscle and/or heart muscle). In specific embodiments, the one or more tissues are or comprise skeletal muscle (e.g., biceps, triceps, quadriceps, tibialis anterior, gastrocnemius, rectus abdominis, diaphragm and/or pectoralis major). In specific embodiments, the one or more tissues are or comprise heart muscle (e.g., heart atrium muscle and/or heart ventricle muscle). In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise muscle (e.g., skeletal muscle (e.g., biceps, triceps, quadriceps, tibialis anterior, gastrocnemius, rectus abdominis, diaphragm and/or pectoralis major) and/or heart muscle (e.g., heart atrium muscle and/or heart ventricle muscle)) and liver. In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)) and brain (e.g., middle front brain, middle rear brain, front brain and/or rear brain). In certain embodiments, the one or more tissues are or comprise liver and brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a recombinant AAV9 particle described herein has a decreased tropism for one or more tissues relative to a wild-type AAV9 particle (preferably, a wild-type AAV9 particle having a capsid protein comprising the amino acid sequence of SEQ ID NO: 2). In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)). In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a recombinant AAV9 particle described herein has a decreased tropism for one or more tissues relative to MyoAAV 1A (preferably, MyoAAV 1A having a capsid protein comprising the amino acid sequence of SEQ ID NO: 3), MyoAAV 2A (preferably, MyoAAV 2A having a capsid protein comprising the amino acid sequence of SEQ ID NO: 4), MyoAAV 3A (preferably, MyoAAV 3A having a capsid protein comprising the amino acid sequence of SEQ ID NO: 111), and/or MyoAAV 4A (preferably, MyoAAV 4A having a capsid protein comprising the amino acid sequence of SEQ ID NO: 5). In certain embodiments, the one or more tissues are or comprise muscle (e.g., skeletal muscle (for example, biceps, triceps, quadriceps, tibialis anterior, gastrocnemius, rectus abdominis, diaphragm and/or pectoralis major) and/or heart muscle (for example, heart atrium muscle and/or heart ventricle muscle)). In certain embodiments, the one or more tissues are or comprise liver. In certain embodiments, the one or more tissues are or comprise brain (e.g., middle front brain, middle rear brain, front brain and/or rear brain). In certain embodiments, the one or more tissues are or comprise kidney. In certain embodiments, the one or more tissues are or comprise lung. In certain embodiments, the one or more tissues are or comprise spleen.

In various embodiments, a recombinant AAV9 particle described herein has an increased tropism for one or more tissues as described above and a decreased tropism for another one or more tissues as described above.

In various embodiments, the tropism for a tissue described herein is measured after a subject is administered systemically with the recombinant AAV9 particle. In certain embodiments, the tropism for a tissue described herein is measured after a subject is administered intravenously with the recombinant AAV9 particle.

In preferred embodiments, a recombinant AAV9 particle described herein is a non-naturally occurring AAV9 particle. In certain embodiments, a recombinant AAV9 particle described herein is isolated. In certain embodiments, a recombinant AAV9 particle described herein is purified.

### Polynucleotides, Vectors and Cells

In another aspect, provided herein is a polynucleotide encoding a variant AAV9 capsid protein described herein (e.g., a variant AAV9 capsid protein described in Section 5.2). Also provided herein are polynucleotides encoding a fragment of a variant AAV9 capsid protein described herein (e.g., a fragment as described in Section 5.2).

In certain embodiments, a polynucleotide encoding a variant AAV9 capsid protein described herein or a fragment thereof comprises the nucleotide sequence of SEQ ID NO: 38.

In certain embodiments, a polynucleotide encoding a variant AAV9 capsid protein described herein or a fragment thereof comprises the nucleotide sequence of SEQ ID NO: 40.

In certain embodiments, a polynucleotide encoding a variant AAV9 capsid protein described herein or a fragment thereof comprises the nucleotide sequence of SEQ ID NO: 42.

In certain embodiments, a polynucleotide encoding a variant AAV9 capsid protein described herein or a fragment thereof comprises the nucleotide sequence of SEQ ID NO: 44.

In certain embodiments, a polynucleotide encoding a variant AAV9 capsid protein described herein or a fragment thereof comprises the nucleotide sequence of SEQ ID NO: 46.

In certain embodiments, a polynucleotide encoding a variant AAV9 capsid protein described herein or a fragment thereof comprises the nucleotide sequence of SEQ ID NO: 48. In certain embodiments, a polynucleotide encoding a variant AAV9 capsid protein described herein or a fragment thereof comprises the nucleotide sequence of SEQ ID NO: 50. In certain embodiments, a polynucleotide encoding a variant AAV9 capsid protein described herein or a fragment thereof comprises the nucleotide sequence of SEQ ID NO: 48 and comprises the nucleotide sequence of SEQ ID NO: 50.

In certain embodiments, a polynucleotide encoding a variant AAV9 capsid protein described herein or a fragment thereof comprises the nucleotide sequence of SEQ ID NO: 52.

In certain embodiments, a polynucleotide encoding a variant AAV9 capsid protein described herein or a fragment thereof comprises the nucleotide sequence of SEQ ID NO: 54.

In certain embodiments, a polynucleotide encoding a variant AAV9 capsid protein described herein or a fragment thereof comprises the nucleotide sequence of SEQ ID NO: 56.

In certain embodiments, a polynucleotide encoding a variant AAV9 capsid protein described herein or a fragment thereof comprises the nucleotide sequence of SEQ ID NO: 58.

In certain embodiments, a polynucleotide encoding a variant AAV9 capsid protein described herein or a fragment thereof comprises the nucleotide sequence of SEQ ID NO: 60. In certain embodiments, a polynucleotide encoding a variant AAV9 capsid protein described herein or a fragment thereof comprises the nucleotide sequence of SEQ ID NO: 62. In certain embodiments, a polynucleotide encoding a variant AAV9 capsid protein described herein or a fragment thereof comprises the nucleotide sequence of SEQ ID NO: 60 and comprises the nucleotide sequence of SEQ ID NO: 62.

In certain embodiments, a polynucleotide encoding a variant AAV9 capsid protein described herein or a fragment thereof comprises the nucleotide sequence of SEQ ID NO: 64.

In certain embodiments, a polynucleotide encoding a variant AAV9 capsid protein described herein or a fragment thereof comprises the nucleotide sequence of SEQ ID NO: 66.

In certain embodiments, a polynucleotide encoding a variant AAV9 capsid protein described herein or a fragment thereof comprises the nucleotide sequence of SEQ ID NO: 68.

In certain embodiments, a polynucleotide encoding a variant AAV9 capsid protein described herein or a fragment thereof comprises the nucleotide sequence of SEQ ID NO: 70.

In certain embodiments, a polynucleotide encoding a variant AAV9 capsid protein described herein or a fragment thereof comprises the nucleotide sequence of SEQ ID NO: 72.

In certain embodiments, a polynucleotide encoding a variant AAV9 capsid protein described herein or a fragment thereof comprises the nucleotide sequence of SEQ ID NO: 74.

In certain embodiments, a polynucleotide encoding a variant AAV9 capsid protein described herein or a fragment thereof comprises the nucleotide sequence of SEQ ID NO: 76.

In certain embodiments, a polynucleotide encoding a variant AAV9 capsid protein described herein or a fragment thereof comprises the nucleotide sequence of SEQ ID NO: 78.

In certain embodiments, a polynucleotide encoding a variant AAV9 capsid protein described herein or a fragment thereof comprises the nucleotide sequence of SEQ ID NO: 100.

In certain embodiments, a polynucleotide encoding a variant AAV9 capsid protein described herein or a fragment thereof comprises the nucleotide sequence of SEQ ID NO: 101.

In certain embodiments, a polynucleotide encoding a variant AAV9 capsid protein described herein or a fragment thereof comprises the nucleotide sequence of SEQ ID NO: 102.

In certain embodiments, a polynucleotide encoding a variant AAV9 capsid protein described herein or a fragment thereof comprises the nucleotide sequence of SEQ ID NO: 103.

In certain embodiments, a polynucleotide encoding a variant AAV9 capsid protein described herein or a fragment thereof comprises the nucleotide sequence of SEQ ID NO: 104.

In certain embodiments, a polynucleotide encoding a variant AAV9 capsid protein described herein or a fragment thereof comprises the nucleotide sequence of SEQ ID NO: 105.

In certain embodiments, a polynucleotide encoding a variant AAV9 capsid protein described herein or a fragment thereof comprises the nucleotide sequence of SEQ ID NO: 106.

In certain embodiments, a polynucleotide encoding a variant AAV9 capsid protein described herein or a fragment thereof comprises the nucleotide sequence of SEQ ID NO: 107.

In certain embodiments, a polynucleotide encoding a variant AAV9 capsid protein described herein or a fragment thereof comprises the nucleotide sequence of SEQ ID NO: 108.

In certain embodiments, the polynucleotide described herein is a DNA polynucleotide. In a specific embodiment, the polynucleotide described herein is a single-stranded DNA. In a specific embodiment, the polynucleotide described herein is a double-stranded DNA. In a specific embodiment, the polynucleotide described herein is a cDNA. In certain embodiments, the polynucleotide described herein is an RNA polynucleotide. In a specific embodiment, the polynucleotide described herein is a single-stranded RNA. In a specific embodiment, the polynucleotide described herein is a double-stranded RNA. In a specific embodiment, the polynucleotide described herein is an mRNA.

In certain embodiments, the polynucleotide described herein is optimized by alternative or preferred codon usage for a particular host cell or delivery target cell type. Codon optimization can be performed using any suitable method known in the art (e.g., using a suitable software known in the art).

In another aspect, provided herein is a vector (e.g., a plasmid, bacmid, cosmid, construct or the like) comprising a polynucleotide described herein, which encodes a variant AAV9 capsid protein described herein or a fragment thereof.

In another aspect, provided herein is a host cell comprising a polynucleotide described herein or a vector described herein. In certain embodiments, the host cell further comprises a recombinant AAV genome comprising a nucleotide sequence encoding a biologic molecule as described in Section 5.3, or a vector (e.g., a plasmid, bacmid, cosmid, construct or the like) encoding such a recombinant AAV genome. In certain embodiments, the host cell further comprises an AAV rep gene-encoding nucleotide sequence, in a polynucleotide or vector (e.g., a plasmid, bacmid, cosmid, construct or the like) that is the same as or different from the variant AAV9 capsid protein-encoding polynucleotide or vector described herein. In certain embodiments, the host cell further comprises helper functions, such as one or more helper plasmid and/or one or more helper virus (see Section 5.5 regarding helper functions).

In another aspect, provided herein is a host cell comprising a recombinant AAV9 particle described herein.

In another aspect, provided herein is a host cell producing a recombinant AAV9 particle described herein.

In some embodiments, the host cell is an ex vivo host cell. In some embodiments, the host cell is an in vitro host cell. In some embodiments, the host cell is an in vivo host cell.

As used herein, the term “host” refers to cells (e.g., cells from insects, animals (including humans and non-human animals), yeast, and bacteria, etc.) which harbor a polynucleotide, vector, AAV particle or AAV genome. It is not intended that the present disclosure be limited to any particular type of host cell. Indeed, it is contemplated that any suitable cell will find use herein as a host. A host cell may be in the form of or be derived from a single cell, a population, a culture (such as a liquid culture or a culture on a solid substrate), a cell line, an organism or part thereof.

Thus, the host cell of the disclosure can be, for example, a bacterial cell, a yeast cell, an insect cell, or a mammalian cell (such as a human cell or a non-human mammalian cell). Non-limiting exemplary insect cells that can be used as host cells include Ao38, High Five, Sf9, Se301, SeIZD2109, SeUCR1, Sf900+, Sf21, BTI-TN-5B1-4, MG-1, Tn368, HzAm1, BM-N, Ha2302 and Hz2E5. Non-limiting exemplary mammalian cells that can be used as host cells include HEK293, HeLa, CHO, NS0, SP2/0, PER. C6, Vero, RD, BHK, HT 1080, A549, Cos-7, ARPE-19 and MRC-5 cells. In certain embodiments, the host cell is a human cell. In specific embodiments, the human cell is autologous to the subject (e.g., a human patient) to be treated by a recombinant AAV9 particle produced by the human cell. In specific embodiments, the human cell is allogeneic to the subject (e.g., a human patient) to be treated by a recombinant AAV9 particle produced by the human cell.

The polynucleotides, vectors and host cells described herein may be used to produce a variant AAV9 capsid protein described herein, a recombinant AAV9 particle described herein, and/or a biologic molecule encoded by the recombinant AAV genome of a recombinant AAV9 particle described herein.

In another aspect, provided herein is a population of host cells stably transduced by a recombinant AAV9 particle described herein. In specific embodiments, the population of host cells are an ex vivo population of host cells. In specific embodiments, the population of host cells are an in vitro population of host cells. In specific embodiments, the population of host cells are an in vivo population of host cells. In specific embodiments, the population of host cells are a population of human host cells. In specific embodiments, the population of host cells are an ex vivo population of human host cells. In specific embodiments, the population of host cells are an in vitro population of human host cells.

In certain embodiments, such a population of host cells may be used to generate a population of recombinant AAV9 particles that are used as a therapeutic and administered to a subject (e.g., a human patient) in need thereof.

In certain embodiments, such a population of host cells may be used to generate a population of biologic molecules encoded by a recombinant AAV genome as described in Section 5.3, which population of biologic molecules is used as a therapeutic and administered to a subject (e.g., a human patient) in need thereof.

In certain embodiments, such a population of host cells may be used as a therapeutic per se and be administered to a subject (e.g., a human patient) in need thereof. In specific embodiments, the population of host cells are autologous to the subject (e.g., a human patient) to be treated by the population of host cells. In specific embodiments, the population of host cells are allogeneic to the subject (e.g., a human patient) to be treated by the population of host cells.

### Methods of Production

In another aspect, provided herein is a method of producing a recombinant AAV9 particle described herein (e.g., a recombinant AAV9 particle described in Section 5.3). Also described herein are methods of producing a polynucleotide described herein (e.g., a polynucleotide described in Section 5.4), a vector described herein (e.g., a vector described in Section 5.4), a host cell described herein (e.g., a host cell described in Section 5.4), a variant AAV9 capsid protein described herein or a fragment thereof (e.g., a variant AAV9 capsid protein described herein or a fragment thereof described in Section 5.2), or a recombinant AAV genome described herein (e.g., a recombinant AAV genome described in Section 5.3).

The polynucleotides, vectors, host cells, variant AAV9 capsid proteins and fragments thereof, recombinant AAV9 particles, and recombinant AAV genomes of the invention may be produced by any suitable means known in the art, including recombinant production, genetic engineering, molecular cloning, chemical synthesis, and other synthetic means. Such production methods are within the knowledge of those of skill in the art and are not a limitation of the present invention.

Generation of a polynucleotide, vector, recombinant AAV genome and/or variant AAV9 capsid protein or fragment thereof of the disclosure may be made using any suitable genetic engineering and protein production techniques known in the art, including, without limitation, cloning, restriction endonuclease digestion, ligation, transformation, plasmid purification, DNA sequencing, chemical synthesis, in vitro translation and in vivo expression, for example as described in Green and Sambrook, Molecular Cloning: A Laboratory Manual, 4^{th} Ed., Cold Spring Harbor Laboratory (Cold Spring Harbor, N.Y. 2012).

A host cell may be made to carry a polynucleotide, vector, recombinant AAV particle or recombinant AAV genome of the disclosure using any suitable method, including, without limitation, transfection, electroporation, transduction, liposome delivery, membrane fusion techniques, high velocity DNA-coated pellets, viral infection and protoplast fusion.

Recombinant AAV particles may be produced by host cells that allow for the production and replication of the AAV particles. Methods of producing recombinant AAV particles are well known in the art and are described in, for example, Adeno-Associated Virus: Methods and Protocols (Methods In Molecular Biology, 280), ed. Snyder and Moullier, Humana Press, NJ (2011); Viral Vectors for Gene Therapy: Methods and Protocols (Methods in Molecular Biology, 1937); ed. Manfredsson and Benskey, Humana Press, NJ (2019); O'Reilly et al., Baculovirus Expression Vectors, A Laboratory Manual, Oxford Univ. Press (1994); Samulski et al., J. Vir. 63: 3822-8 (1989); Kajigaya et al., Proc. Nat'l. Acad. Sci. USA 88: 4646-50 (1991); Ruffing et al., J. Vir. 66: 6922-30 (1992); Kimbauer et al., Vir. 219: 37-44 (1996); Zhao et al., Vir. 272: 382-93 (2000); U.S. Pat. Nos. 5,064,764, 5,756,283, 6,194,191, 6,204,059, 6,258,595, 6,261,551, 6,270,996, 6,281,010, 6,365,394, 6,475,769, 6,482,634, 6,485,966, 6,566,118, 6,943,019, 6,953,690, 7,022,519, 7,238,526, 7,291,498, 7,491,508 and 8,137,948; and International Patent Application Publication Nos. WO1996039530, WO1998010088, WO1999014354, WO1999015685, WO1999047691, WO2000055342, WO2000075353, WO2001023597, WO2015191508, WO2018022608, WO2019217513, WO2019222132, WO2019222136 and WO2020232044; the disclosures of each of which are incorporated herein by reference in their entirety.

AAV viruses grow only in cells in which helper functions for generating a productive AAV infection are present. In certain embodiments, helper functions are provided by one or more helper plasmids and/or helper viruses (e.g., adenoviruses, baculoviruses, vaccinia viruses, herpesviruses, or papillomaviruses) comprising helper genes. A skilled person will appreciate that any helper virus and/or helper plasmid that can provide adequate helper function to AAV can be used herein. Non-limiting examples of the adenoviral or baculoviral helper genes include, but are not limited to, ElA, ElB, E2A, E4 and VA.

AAV cap gene product and AAV rep gene product are also required for AAV viruses to generate a productive AAV infection. In various embodiments, the AAV cap gene product is supplied in trans. In other words, the AAV genome does not comprise the AAV cap gene. In various embodiments, the AAV rep gene product is supplied in trans. In other words, the AAV genome does not comprise the AAV rep gene. In various embodiments, the AAV cap gene product and the AAV rep gene product are both supplied in trans. In other words, the AAV genome does not comprise the AAV cap gene and does not comprise the AAV rep gene. In certain embodiments, the AAV cap gene is present in a vector (e.g., a plasmid, bacmid, cosmid, construct or the like) that is transfected into a host cell comprising other necessary components (e.g., a vector (e.g., a plasmid, bacmid, cosmid, construct or the like) encoding a recombinant AAV genome comprising a biologic molecule-encoding nucleotide sequence of interest flanked by ITRs), AAV rep gene, and helper functions) to produce infectious AAV particles. In certain embodiments, the AAV rep gene is present in a vector (e.g., a plasmid, bacmid, cosmid, construct or the like) that is transfected into a host cell comprising other necessary components (e.g., a vector (e.g., a plasmid, bacmid, cosmid, construct or the like) encoding a recombinant AAV genome comprising a biologic molecule-encoding nucleotide sequence of interest flanked by ITRs), AAV cap gene, and helper functions) to produce infectious AAV particles. In certain embodiments, the AAV cap gene and the AAV rep gene are present in one or two vectors (e.g., plasmid (s), bacmid (s), cosmid (s), construct (s) or the like) that are transfected into a host cell comprising other necessary components (e.g., a vector (e.g., a plasmid, bacmid, cosmid, construct or the like) of a recombinant AAV genome comprising a biologic molecule-encoding nucleotide sequence of interest flanked by ITRs) and helper functions) to produce infectious AAV particles.

### Pharmaceutical Compositions

In another aspect, provided herein is a pharmaceutical composition comprising a recombinant AAV9 particle described herein (e.g., a recombinant AAV9 particle described in Section 5.3) and a pharmaceutically acceptable carrier.

In another aspect, provided herein is a pharmaceutical composition comprising a population of host cells described herein that are stably transduced by a recombinant AAV9 particle described herein (e.g., a population of host cells stably transduced by a recombinant AAV9 particle as described in Section 5.4) and a pharmaceutically acceptable carrier.

In certain embodiments, the concentration of a recombinant AAV9 particle in a pharmaceutical composition described herein may range from 1×10⁸ vg/ml to 1×10²⁰ vg/ml. In specific embodiments, the concentration of a recombinant AAV9 particle in a pharmaceutical composition described herein may range from 1×10⁹ vg/ml to 1×10¹⁹ vg/ml. In specific embodiments, the concentration of a recombinant AAV9 particle in a pharmaceutical composition described herein may range from 1×10¹⁰ vg/ml to 1×10¹⁸ vg/ml. In specific embodiments, the concentration of a recombinant AAV9 particle in a pharmaceutical composition described herein may range from 1×10¹¹ vg/ml to 1×10¹⁷ vg/ml. In specific embodiments, the concentration of a recombinant AAV9 particle in a pharmaceutical composition described herein may range from 1×10¹² vg/ml to 1×10¹⁶ vg/ml. In specific embodiments, the concentration of a recombinant AAV9 particle in a pharmaceutical composition described herein may range from 1×10¹³ vg/ml to 1×10¹⁵ vg/ml.

In certain embodiments, the concentration of host cells in a pharmaceutical composition described herein may range from 1×10² cells/ml to 1×10¹² cells/ml. In specific embodiments, the concentration of host cells in a pharmaceutical composition described herein may range from 1×10³ cells/ml to 1×10¹¹ cells/ml. In specific embodiments, the concentration of host cells in a pharmaceutical composition described herein may range from 1×10⁴ cells/ml to 1×10¹⁰ cells/ml. In specific embodiments, the concentration of host cells in a pharmaceutical composition described herein may range from 1×10⁵ cells/ml to 1×10⁹ cells/ml. In specific embodiments, the concentration of host cells in a pharmaceutical composition described herein may range from 1×10⁶ cells/ml to 1×10⁸ cells/ml.

Typically, an agent (e.g., an excipient or carrier) is pharmaceutically acceptable when it is safe, non-toxic, and is not biologically or otherwise undesirable, and is acceptable for veterinary use as well as human pharmaceutical use.

In certain embodiments, a pharmaceutical composition described herein comprises one or more pharmaceutically acceptable excipients to provide the composition with advantageous properties for storage and/or administration to subjects for the treatment of a disease or disorder. In certain embodiments, a pharmaceutical composition described herein comprises one or more buffering agents, such as sodium phosphate dibasic and/or sodium phosphate monobasic monohydrate. In certain embodiments, a pharmaceutical composition described herein comprises one or more isotonicity agents, such as sodium chloride. In certain embodiments, a pharmaceutical composition described herein comprises one or more bulking agents, such as mannitol, sucrose, dextran, lactose, trehalose, and/or povidone (PVP K24). In certain embodiments, a pharmaceutical composition described herein comprises one or more surfactants, such as polysorbate 80, polysorbate 20, sodium dodecyl sulfate, sodium stearate, ammonium lauryl sulfate, TRITON AG 98 (Rhone-Poulenc), poloxamer 407, and/or poloxamer 188.

Preferably, the pharmaceutical compositions described herein are stable and can be stored for extended periods of time without an unacceptable change in quality, potency, or purity, for example, at below −60° C., at about −20° C., at about 2° C. to 8° C., and/or at room temperature.

Preferably, the pharmaceutical compositions described herein are sterile and stable under the conditions of manufacture and storage. Pharmaceutical compositions described herein may be formulated as a solution, microemulsion, liposome, lyophilized composition, or other ordered structure suitable to accommodate high drug concentration.

In certain embodiments, a pharmaceutical composition described herein is formulated for a route of administration to a subject. Non-limiting examples of routes of administration that can be used include direct delivery to the target organ, oral, inhalation, intravenous, intramuscular, subcutaneous, intradermal, intranasal, intrathecal, intrapancreatic, intraperitoneal, intratumoral, and other parental routes of administration. In specific embodiments, a pharmaceutical composition described herein is formulated for systemic administration to a subject. In a specific embodiment, a pharmaceutical composition described herein is formulated for intravenous administration to a subject.

### Methods of Delivery and Treatment

In another aspect, provided herein is a method of delivering a biologic molecule to one or more ex vivo target cells, comprising transducing the one or more target cells with a recombinant AAV9 particle described herein (e.g., a recombinant AAV9 particle described in Section 5.3).

In another aspect, provided herein is a method of delivering a biologic molecule to one or more in vitro target cells, comprising transducing the one or more target cells with a recombinant AAV9 particle described herein (e.g., a recombinant AAV9 particle described in Section 5.3).

In another aspect, provided herein is a method of delivering a biologic molecule to one or more in vivo target cells in a subject, comprising administering to the subject a recombinant AAV9 particle described herein (e.g., a recombinant AAV9 particle described in Section 5.3).

In another aspect, provided herein is a method of delivering a biologic molecule to one or more in vivo target cells in a subject, comprising administering to the subject a pharmaceutical composition described herein (e.g., a pharmaceutical composition described in Section 5.6).

The biologic molecule can be, for example, but not limited to, a polypeptide, a protein, a nucleic acid (e.g., DNA or RNA), or an oligonucleotide (e.g., siRNA, shRNA, miRNA or aptamer). In specific embodiments, the biologic molecule is a polypeptide or a protein. In preferred embodiments, the biologic molecule is a human polypeptide or a human protein. The biologic molecule can be a reporter molecule, such as a reporter protein (for example, a fluorescent protein (e.g., green fluorescent protein (GFP)), luciferase (e.g., firefly luciferase), β-lactamase, or β-galactosidase (LacZ)). The biologic molecule can also be a therapeutic molecule, such as a therapeutic protein. A therapeutic molecule may be used to correct or ameliorate gene deficiencies associated with a disease or disorder. Exemplary therapeutic molecules may include, without limitation, enzymes, cytokines, growth factors, kinases, dominant negative mutant proteins, antibodies and antigen-binding fragments thereof, and interleukins. In specific embodiments, the biologic molecule is expressed in muscle cells (e.g., expressed at a higher level in muscle cells relative to other cell types). In specific embodiments, the biologic molecule is expressed in liver cells (e.g., expressed at a higher level in liver cells relative to other cell types). In specific embodiments, the biologic molecule is expressed in brain cells (e.g., expressed at a higher level in brain cells relative to other cell types). In specific embodiments, the biologic molecule is expressed in both muscle cells and liver cells (e.g., expressed at higher level in both muscle cells and liver cells relative to other cell types). In specific embodiments, the biologic molecule is expressed in both muscle cells and brain cells (e.g., expressed at higher level in both muscle cells and brain cells relative to other cell types). In specific embodiments, the biologic molecule is expressed in both liver cells and brain cells (e.g., expressed at higher level in both liver cells and brain cells relative to other cell types). In specific embodiments, the biologic molecule is expressed in kidney cells (e.g., expressed at a higher level in kidney cells relative to other cell types). In specific embodiments, the biologic molecule is expressed in lung cells (e.g., expressed at a higher level in lung cells relative to other cell types). In specific embodiments, the biologic molecule is expressed in spleen cells (e.g., expressed at a higher level in spleen cells relative to other cell types). In specific embodiments, the biologic molecule functions in muscle cells (e.g., is required or important for the normal function of muscle cells). In specific embodiments, the biologic molecule functions in liver cells (e.g., is required or important for the normal function of liver cells). In specific embodiments, the biologic molecule functions in brain cells (e.g., is required or important for the normal function of brain cells). In specific embodiments, the biologic molecule functions in both muscle cells and liver cells (e.g., is required or important for the normal function of both muscle cells and liver cells). In specific embodiments, the biologic molecule functions in both muscle cells and brain cells (e.g., is required or important for the normal function of both muscle cells and brain cells). In specific embodiments, the biologic molecule functions in both liver cells and brain cells (e.g., is required or important for the normal function of both liver cells and brain cells). In specific embodiments, the biologic molecule functions in kidney cells (e.g., is required or important for the normal function of kidney cells). In specific embodiments, the biologic molecule functions in lung cells (e.g., is required or important for the normal function of lung cells). In specific embodiments, the biologic molecule functions in spleen cells (e.g., is required or important for the normal function of spleen cells). In specific embodiments, the muscle cells described herein are skeletal muscle cells (e.g., biceps, triceps, quadriceps, tibialis anterior, gastrocnemius, rectus abdominis, diaphragm and/or pectoralis major cells) and/or heart muscle cells (e.g., heart atrium muscle and/or heart ventricle muscle cells). In specific embodiments, the brain cells described herein are middle front brain, middle rear brain, front brain and/or rear brain cells. In a specific embodiment, the biologic molecule is a functional Survival Motor Neuron (SMN) protein (e.g., a wild-type SMN protein). In a specific embodiment, the biologic molecule is a functional microdystrophin (e.g., a wild-type microdystrophin). In a specific embodiment, the biologic molecule is a functional alpha-galactosidase (e.g., a wild-type alpha-galactosidase). In a specific embodiment, the biologic molecule is a functional phenylalanine hydroxylase (PAH) (e.g., a wild-type PAH). In a specific embodiment, the biologic molecule is a functional Coagulation Factor VIII (FVIII) (e.g., a wild-type FVIII). In a specific embodiment, the biologic molecule is a functional Coagulation Factor IX (FIX) (e.g., a wild-type FIX). In a specific embodiment, the biologic molecule is a functional beta-glucocerebrosidase (GBA) (e.g., a wild-type GBA). In a specific embodiment, the biologic molecule is NPC Intracellular Cholesterol Transporter 1 (NPC1) (e.g., a wild-type NPC1). In a specific embodiment, the biologic molecule is NPC Intracellular Cholesterol Transporter 2 (NPC2) (e.g., a wild-type NPC2). In a specific embodiment, the biologic molecule is acid alpha-glucosidase (GAA) (e.g., a wild-type GAA).

In certain embodiments, the one or more target cells are one or more muscle cells. In certain embodiments, the one or more target cells are one or more liver cells. In certain embodiments, the one or more target cells are one or more brain cells. In certain embodiments, the one or more target cells are one or more muscle cells and one or more liver cells. In certain embodiments, the one or more target cells are one or more muscle cells and one or more brain cells. In certain embodiments, the one or more target cells are one or more liver cells and one or more brain cells. In certain embodiments, the one or more target cells are one or more muscle cells, one or more liver cells, and/or one or more brain cells. In certain embodiments, the one or more target cells are one or more kidney cells. In certain embodiments, the one or more target cells are one or more lung cells. In certain embodiments, the one or more target cells are one or more spleen cells. In certain embodiments, the one or more target cells are one or more muscle cells, one or more liver cells, one or more brain cells, one or more kidney cells, one or more lung cells, and/or one or more spleen cells. In specific embodiments, the one or more muscle cells described herein are one or more skeletal muscle cells (e.g., biceps, triceps, quadriceps, tibialis anterior, gastrocnemius, rectus abdominis, diaphragm and/or pectoralis major cell (s)) and/or one or more heart muscle cells (e.g., heart atrium muscle and/or heart ventricle muscle cell (s)). In specific embodiments, the one or more brain cells described herein are one or more middle front brain, middle rear brain, front brain and/or rear brain cell (s).

In another aspect, provided herein is a method of treating a disease or disorder in a subject in need thereof, comprising administering to the subject, preferably a therapeutically effective amount of, a recombinant AAV9 particle described herein (e.g., a recombinant AAV9 particle described in Section 5.3).

In another aspect, provided herein is a method of treating a disease or disorder in a subject in need thereof, comprising administering to the subject, preferably a therapeutically effective amount of, a pharmaceutical composition described herein (e.g., a pharmaceutical composition described in Section 5.6).

The route of administration or delivery and the amount of the recombinant AAV9 particle or pharmaceutical composition to be administered to a subject can be determined based on the nature of the disease or disorder, condition of the subject and the knowledge of the physician. Non-limiting examples of routes of administration or delivery that can be used include direct delivery to the target organ, oral, inhalation, intravenous, intramuscular, subcutaneous, intradermal, intranasal, intrathecal, intrapancreatic, intraperitoneal, intratumoral, and other parental routes of administration. In a specific embodiment, a recombinant AAV9 particle or a pharmaceutical composition described herein is administered or delivered systemically. In a specific embodiment, a recombinant AAV9 particle or a pharmaceutical composition described herein is administered or delivered intravenously.

The methods of delivery and methods of treatment described herein can be used to treat various diseases or disorders, including, without limitation, cancers (such as carcinoma, sarcoma, leukemia, lymphoma, germ cell tumors, and blastoma), autoimmune diseases, infectious diseases, and genetic disorders. In specific embodiments, the disease or disorder is caused at least by dysfunction of the muscle (e.g., skeletal muscle (e.g., biceps, triceps, quadriceps, tibialis anterior, gastrocnemius, rectus abdominis, diaphragm and/or pectoralis major) and/or heart muscle (e.g., heart atrium muscle and/or heart ventricle muscle)). In specific embodiments, the disease or disorder is caused at least by dysfunction of the liver. In specific embodiments, the disease or disorder is caused at least by dysfunction of the brain (e.g., middle front brain, middle rear brain, front brain and/or rear brain). In specific embodiments, the disease or disorder is caused at least by dysfunction of the muscle (e.g., skeletal muscle (e.g., biceps, triceps, quadriceps, tibialis anterior, gastrocnemius, rectus abdominis, diaphragm and/or pectoralis major) and dysfunction of the liver. In specific embodiments, the disease or disorder is caused at least by dysfunction of the muscle (e.g., skeletal muscle (e.g., biceps, triceps, quadriceps, tibialis anterior, gastrocnemius, rectus abdominis, diaphragm and/or pectoralis major) and dysfunction of the brain (e.g., middle front brain, middle rear brain, front brain and/or rear brain). In specific embodiments, the disease or disorder is caused at least by dysfunction of the liver and dysfunction of the brain (e.g., middle front brain, middle rear brain, front brain and/or rear brain). In specific embodiments, the disease or disorder is caused at least by dysfunction of the kidney. In specific embodiments, the disease or disorder is caused at least by dysfunction of the lung. In specific embodiments, the disease or disorder is caused at least by dysfunction of the spleen. In a specific embodiment, the disease or disorder is spinal muscular atrophy. In a specific embodiment, the disease or disorder is Duchenne muscular dystrophy (DMD). In a specific embodiment, the disease or disorder is Fabry disease. In a specific embodiment, the disease or disorder is Gaucher disease. In a specific embodiment, the disease or disorder is Niemann-Pick disease type C (NPC). In a specific embodiment, the disease or disorder is Pompe disease. In a specific embodiment, the disease or disorder is phenylalanine hydroxylase deficiency. In a specific embodiment, the disease or disorder is hemophilia A. In a specific embodiment, the disease or disorder is hemophilia B.

### Sequences

TABLE 1
Table of Sequences.

| SEQ | | |
|---|---|---|
| ID | | |
| NO. | Description | SEQUENCE |
| 1 | Rep2-AAP | ggtcctgtattagaggtcacgtgagtgttttgcgacattttgcgacaccatgtggtcacgctgggtatttaagcccgagtga |
| | | gcacgcagggtctccattttgaagcgggaggtttgaacgcgcagccaccacggcggggttttacgagattgtgattaag |
| | | gtccccagcgaccttgacgagcatctgcccggcatttctgacagctttgtgaactgggtggccgagaaggaatgggag |
| | | ttgccgccagattctgacatggatctgaatctgattgagcaggcacccctgaccgtggccgagaagctgcagcgcgac |
| | | tttctgacggnatggcgccgtgtgagtaaggccccggaggcccttttctttgtgcaatttgagaagggagagagctactt |
| | | ccacatgcacgtgctcgtggaaaccaccggggtgaaatccatggttttgggacgtttcctgagtcagattcgcgaaaaa |
| | | ctgattcagagaatttaccgcgggatcgagccgactttgccaaactggttcgcggtcacaaagaccagaaatggcgcc |
| | | ggaggcgggaacaaggtggtggatgagtgctacatccccaattacttgctccccaaaacccagcctgagctccagtgg |
| | | gcgtggactaatatggaacagtatttaagcgcctgtttgaatctcacggagcgtaaacggttggtggcgcagcatctgac |
| | | gcacgtgtcgcagacgcaggagcagaacaaagagaatcagaatcccaattctgatgcgccggtgatcagatcaaaaa |
| | | cttcagccaggtacatggagctggtcgggtggctcgtggacaaggggattacctcggagaagcagtggatccaggag |
| | | gaccaggcctcatacatctccttcaatgcggcctccaactcgcggtcccaaatcaaggctgccttggacaatgcgggaa |
| | | agattatgagcctgactaaaaccgcccccgactacctggtgggccagcagcccgtggaggacatttccagcaatcgga |
| | | tttataaaattttggaactaaacgggtacgatccccaatatgcggcttccgtctttctgggatgggccacgaaaaagttog |
| | | gcaagaggaacaccatctggctgtttgggcctgcaactaccgggaagaccaacatcgcggaggccatagcccacact |
| | | gtgcccttctacgggtgcgtaaactggaccaatgagaactttcccttccaacgactgtgtcgacaagatggtgatctggtg |
| | | ggaggaggggaagatgaccgccaaggtcgtggagtcggccaaagccattctcggaggaagcaaggtgcgcgtgga |
| | | ccagaaatgraagtcctcggcccagatagacccgactcccgtgatcgtcacctccaacaccaacatgtgcgccgtgatt |
| | | gacgggaactcaacgaccttcgaacaccagcagccgttgcaagaccggatgttcaaatttgaactcacccgccgtctg |
| | | gatcatgactttgggaaggtcaccaagcaggaagtcaaagactttttccggtgggcaaaggatcacgtggttgaggtgg |
| | | agcatgaattctacgtcaaaaagggggagccaagaaaagacccgcccccagtgacgcagatataagtgagcccaaa |
| | | cgggtgcgcgagtcagttgcgcagccatcgacgtcagacgcggaagcttcgatcaactacgcagacaggtaccaaaa |
| | | caaatgttctcgtcacgtgggcatgaatctgatgctgtttccctgcagacaatgcgagagaatgaatcagaattcaaatat |
| | | ctgcttcactcacggacagaaagactgtttagagtgctttcccgtgtcagaatctcaacccgtttctgtcgtcaaaaaggc |
| | | gtatcagaaactgtgctacattcatcatatcatgggaaaggtgccagacgcttgcactgcctgcgatctggtcaatgtgga |
| | | tttggatgactgcatctttgaacaataaatgatttaaatcaggtatggctgccgatggttaacttccagattgactcgaggac |
| | | aaccttagtgaaggaattcgcgagtggtgggctttgaaacctggagcccctcaacccaaggcaaatcaacaacatcaa |
| | | gacaacgctcgaggtcttgtgcttccgggttacaaataccttggacccggcaacggactcgacaagggggagccggt |
| | | caacgcagcagadgcggcggccctcgagcacgacaaggcctacgaccagcagctcaaggccggagacaacccgt |
| | | acctcaagtacaaccacgccgacgccgagttccaggagcggctcaaagaagatacgtcttttgggggcaacctcggg |
| | | cgagcagtcttccaggccaaaaagaggcttcttgaacctcttggtctggttgaggaagcggctaagacggctcctggat |
| | | agaagaggcctgtagagtagtctcctcaggaaccggactcctccgcgggtattggcaaatcgggtgcacagcccgcta |
| | | aaaagagactcaatttcggtcagactggcgacacagagtcagtcccagaccctcaaccaatcggagaacctcccgca |
| | | gccccctcaggtgtgggatctcttacaatggcttcaggtggtggcgcaccagtggcagacaataactaaggtgccgatg |
| | | gagtgggtagttcctogggaaattggcattgcgattcccaatggctgggggacagagtcatcaccaccagcacccgaa |
| | | cctgggccctgcccacctacaacaatcacctctacaagcaaatctccaacagcacatctggaggatcttcaaatgacaa |
| | | cgcctacttcggctacagcaccccctgggggtattttgacttcaacagattccactgccacttctcaccacgtgactggca |
| | | gcgactcatcaacaacaactggggattccggcctaagcgactcaacttcaagctcttcaacattcaggtcaaagaggtta |
| | | cggacaacaatggagtcaagaccatcgccaataaccttaccagcacggtccaggtcttcacggactcagactatcagct |
| | | cccgtacgtgctcgggtcggctcacgagggctgcctcccgccgttcccagcggacgttttcatgattcctcagtacggg |
| | | tatctgacgcttaatgatggaagccaggccgtgggtcgttcgtccttttactgcctggaatatttcccgtcgcaaatgctaa |
| | | gaacgggtaacaacttccagttcagctacgagtttgagaacgtaccttccatagcagctacgctcacagccaaagcctg |
| | | gaccgactaatgaatccactcatcgaccaatacttgtactatctctcaaagaccggctgggttcaaaaccaaggaatactt |
| | | ccgggtatggtttggcaggacagagatgtgtacctgcaaggacccatttgggccaaaattcctcacacggacggcaac |
| | | tttcacccttctccgctgatgggagggtttggaatgaagcacccgcctcctcagatcctcatcaaaaacacacctgtacct |
| | | gcggatcctccaacggccttcaacaaggacaagctgaactctttcatcacccagtattctactggccaagtcagcgtgga |
| | | gatcgagtgggagctgcagaaggaaaacagcaagcgctggaacccggagatccagtacacttccaactattacaagt |
| | | ctaataatgttgaatttgctgttaatactgaaggtgtatatagtgaaccccgccccattggcaccagatacctgactcgtaat |
| | | ctgtaa |
| 2 | wild-type AAV9 VP1 amino acid | MAADGYLPDWLEDNLSEGIREWWALKPGAPQPKANQQIIQDNARGLVLP |
| | sequence | GYKYLGPGNGLDKGEPVNAADAAALEIIDKAYDQQLKAGDNPYLKYNIIA |
| | (underlined: position 586-588) | DAEFQERLKEDTSFGGNLGRAVFQAKKRLLEPLGLVEEAAKTAPGKKRPV |
| | | EQSPQEPDSSAGIGKSGAQPAKKRLNFGQTGDTESVPDPQPIGEPPAAPSGV |
| | | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQSAQAQAQTGWVQNQGIL |
| | | PGMVWQDRDVYLQGPIWAKIPHTDGNFHPSPLMGGFGMKHPPPQILIKNTP |
| | | VPADPPTAFNKDKLNSFITQYSTGQVSVEIEWELQKENSKRWNPEIQYTSN |
| | | YYKSNNVEFAVNTEGVYSEPRPIGTRYLTRNL |
| 178 | wild-type AAV9 VP2 amino acid | TAPGKKRPVEQSPQEPDSSAGIGKSGAQPAKKRLNFGQTGDTESVPDPQPIG |
| | sequence | EPPAAPSGVGSLTMASGGGAPVADNNEGADGVGSSSGNWHCDSQWLGDR |
| | | VITTSTRTWALPTYNNHLYKQISNSTSGGSSNDNAYFGYSTPWGYFDFNRF |
| | | IICHFSPRDWQRLINNNWGFRPKRLNFKLFNIQVKEVTDNNGVKTIANNLTS |
| | | TVQVFTDSDYQLPYVLGSAIIEGCLPPFPADVFMIPQYGYLTLNDGSQAVGR |
| | | SSFYCLEYFPSQMLRTGNNFQFSYEFENVPFIISSYAIISQSLDRLMNPLIDQY |
| | | LYYLSKTINGSGQNQQTLKFSVAGPSNMAVQGRNYIPGPSYRQQRVSTTVT |
| | | QNNNSEFAWPGASSWALNGRNSLMNPGPAMASHKEGEDRFFPLSGSLIFG |
| | | KQGTGRDNVDADKVMITNEEEIKTINPVATESYGQVATNHQSAQAQAQT |
| | | GWVQNQGILPGMVWQDRDVYLQGPIWAKIPHTDGNFHPSPLMGGFGMKH |
| | | PPPQILTKNTPVPADPPTAFNKDKLNSFITQYSTGQVSVEIEWELQKENSKR |
| | | WNPEIQYTSNYYKSNNVEFAVNTEGVYSEPRPIGTRYLTRNL |
| 179 | wildstype AAV9 VP3 amino acid | MASGGGAPVADNNEGADGVGSSSGNWHCDSQWLGDRVITTSTRTWALPT |
| | Sequence | YNNHLYKQISNSTSGGSSNDNAYFGYSTPWGYFDFNRFHCHFSPRDWQRLI |
| | | NNNWGFRPKRLNFKLFNIQVKEVTDNNGVKTIANNLISTVQVFTDSDYQL |
| | | PYVLGSAHEGCLPPFPADVFMIPQYGYLTLNDGSQAVGRSSFYCLEYFPSQ |
| | | MLRTGNNFQFSYEFENVPFIISSYAIISQSLDRLMNPLIDQYLYYLSKTINGS |
| | | GQNQQTLKFSVAGPSNMAVQGRNYIPGPSYRQQRVSTTVTQNNNSEFAWP |
| | | GASSWALNGRNSLMNPGPAMASIIKEGEDRFFPLSGSLIFGKQGTGRDNVD |
| | | ADKVMITNEEEIKTINPVATESYGQVATNHQSAQAQAQTGWVQNQGILPG |
| | | MVWQDRDVYLQGPIWAKIPHTDGNFHPSPLMGGFGMKHPPPQILIKNTPVP |
| | | ADPPTAFNKDKLNSFITQYSTGQVSVEIEWELQKENSKRWNPEIQYTSNYY |
| | | KSNNVEFAVNTEGVYSEPRPIGTRYLTRNL |
| 3 | published MyoAAV 1A VP1 amino acid | MAADGYLPDWLEDNLSEGIREWWALKPGAPQPKANQQHQDNARGLVLP |
| | sequence (underlined: corresponding to | GYKYLGPGNGLDKGEPVNAADAAALEHDKAYDQQLKAGDNPYLKYNHA |
| | position 586-588 of wild-type AAV9 | DAFFQERLKEDTSFGGNLGRAVFQAKKRLLEPLGLVERAAKTAPGKKRPV |
| | VP1 amino acid sequence; italic and | EQSPQEPDSSAGIGKSGAQPAKKRLNFGQTGDTESVPDPQPIGEPPAAPSGV |
| | underlined: insert after wild-type | GSLTMASGGGAPVADNNEGADGVGSSSGNWHCDSQWLGDRVITTSTRTW |
| | position 588) | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQSAQRGDLTTPAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 34 | published MyoAAV 1A VP1's insert | RGDLTTP |
| | peptide amino acid sequence | |
| 109 | published MyoAAV 1A VP1's amino | SAQRGDLTTP |
| | acid sequence corresponding to position | |
| | 586-588 of wild-type AAV9 VP1 plus | |
| | insert peptide | |
| 4 | published MyoAAV 2A VP1 amino acid | MAADGYLPDWLEDNLSEGIREWWALKPGAPQPKANQQHQDNARGLVLP |
| | sequence (underlined: corresponding to | GYKYLGPGNGLDKGEPVNAADAAALEHDKAYDQQLKAGDNPYLKYNHA |
| | position 586-588 of wild-type AAV9 | DAEFQERLKEDTSFGGNLGRAVFQAKKRLLEPLGLVEEAAKTAPGKKRPV |
| | VP1 amino acid sequence; italic and | EQSPQEPDSSAGIGKSGAQPAKKRLNFGQTGDIESVPDPQPIGEPPAAPSGV |
| | underlined: insert after wild-type | GSLTMASGGGAPVADNNEGADGVGSSSGNWIICDSQWLGDRVITTSTRTW |
| | position 588) | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLILNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTINPVATESYGQVATNHQGPGRGDQTTLAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 35 | published MyoAAV 2A VP1's insert | RGDQTTL |
| | peptide amino acid sequence | |
| 110 | published MyoAAV 2A VP1's amino | GPGRGDQTTL |
| | acid sequence corresponding to position | |
| | 586-588 of wild-type AAV9 VP1 plus | |
| | insert peptide | |
| 111 | published MyoAAV 3A VP1 amino acid | MAADGYLPDWLEDNLSEGIREWWALKPGAPQPKANQQHQDNARGLVLP |
| | sequence (underlined: corresponding to | GYKYLGPGNGLDKGEPVNAADAAALEIIDKAYDQQLKAGDNPYLKYNIIA |
| | position 586-588 of wild-type AAV9 | DAEFQERLKEDTSFGGNLGRAVFQAKKRLLEPLGLVEEAAKTAPGKKRPV |
| | VP1 amino acid sequence; italic and | EQSPQEPDSSAGIGKSGAQPAKKRLNFGQTGDTESVPDPQPIGEPPAAPSGV |
| | underlined: insert after wild-type | GSLTMASGGGAPVADNNEGADGVGSSSGNWHCDSQWLGDRVITTSTRTW |
| | position 588) | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTINPVATESYGQVAINHQSAQRGDYVGLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 112 | published MyoAAV 3A VP1's insert | RGDYVGL |
| | peptide amino acid sequence | |
| 113 | published MyoAAV 3A VP1's amino | SAQRGDYVGL |
| | acid sequence corresponding to position | |
| | 586-588 of wild-type AAV9 VP1 plus | |
| | insert peptide | |
| 5 | published MyoAAV 4A VP1 amino acid | MAADGYLPDWLEDNLSEGIREWWALKPGAPQPKANQQHQDNARGLVLP |
| | sequence (underlined: corresponding to | GYKYLGPGNGLDKGEPVNAADAAALEHDKAYDQQLKAGDNPYLKYNHA |
| | position 586-588 of wild-type AAV9 | DAEFQERLKEDTSFGGNLGRAVFQAKKRLLEPLGLVEEAAKTAPGKKRPV |
| | VP1 amino acid sequence; italic and | EQSPQEPDSSAGIGKSGAQPAKKRLNFGQTGDTESVPDPQPIGEPPAAPSGV |
| | underlined: insert after wild-type | GSLTMASGGGAPVADNNEGADGVGSSSGNWIICDSQWLGDRVITTSTRTW |
| | position 588) | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPQPAMASHKEGEDRFFPLSGSLIFGKQGTGRDN |
| | | VDADKVMITNEEEIKTTNPVATESYGQVATNHQSNSRGDYNSLAQAQTGW |
| | | VQNQGILPGMYWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 36 | published MyoAAV 4A VP1's insert | RGDYNSL |
| | peptide amino acid sequence | |
| 114 | published MyoAAV 4A VP1's amino | SNSRGDYNSL |
| | acid sequence corresponding to position | |
| | 586-588 of wild-type AAV9 VP1 plis | |
| | insert peptide | |
| 6 | AVT901 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italie: insert after wild- | EQSPQEPDSSAGIGKSGAQPAKKRLNFGQTGDTESVPDPQPIGEPPAAPSGV |
| | type position 453) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLILNDGSQAVGRSSFYCLEYF |
| | | PSQMLRTGNNFQFSYEFENVPFHSSYAHSQSLDRLMNPLIDQYLYYLSKTIN |
| | | GHMNGRNNSGQNQQTLKESVAGPSNMAVQGRNYIPGPSYRQQRVSTTVTQ |
| | | NNNSEFAWPGASSWALNGRNSLMNPGPAMASHKEGEDRFFPLSGSLIFGK |
| | | QGTGRDNVDADKVMITNEEFIKTTNPVATESYGQVATNHQSAQAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 37 | AVT901 VP1's insert peptide amino | HMNGRNN |
| | acid sequence | |
| 38 | nucleotide sequence encoding AVT901 | CATATGAATGGGCGTAATAAT |
| | VP1's insert peptide | |
| 7 | AVT902 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic: insert after wild- | EQSPQEPDSSAGIGKSGAQPAKKRLNFGQTGDTESVPDPQPIGEPPAAPSGV |
| | type position 453) | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GPHYTHGISGQNQQTLKFSVAGPSNMAVQGRNYIPGPSYRQQRVSTTVTQN |
| | | NNSEFAWPGASSWALNGRNSLMNPGPAMASHKEGEDRFFPLSGSLIFGKQ |
| | | GTGRDNVDADKVMITNEEEIKTTNPVATESYGQVATNHQSAQAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVFIEWELQKENSKRWNP |
| | | FIQYTSNYYKSNNVEFAVNTEGVYSEPRPIGTRYLTRNL |
| 39 | AVT902 VP1's insert peptide amino | PHYTHGI |
| | acid sequence | |
| 40 | nucleotide sequence encoding AVT902 | cctcattatacgcatggtatt |
| | VP1's insert peptide | |
| 8 | AVT903 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVEQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAHIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQSAQRGDTNNTNMAQAQT |
| | | GWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKR |
| | | WNPEIQYTSNYYKSNNVEFAVNTEGVYSEPRPIGTRYLTRNL |
| 41 | AVT903 VP1's insert peptide amino | RGDINNINM |
| | acid sequence | |
| 42 | nucleotide sequence encoding AVT903 | aggggtgatactaataatactaatatg |
| | VP1's insert peptide | |
| 169 | AVT903 VP1's amino acid sequence | SAQRGDTNNTNM |
| | corresponding to position 586-588 of | |
| | wild-type AAV9 VP1 plus insert peptide | |
| 9 | AVT905 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADQVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQSAQSRGDRPMAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVFIEWELQKENSKRWNP |
| | | EIQYTSNYYKSNNVEFAVNTEGVYSEPRPIGTRYLTRNL |
| 43 | AVT905 VP1's insert peptide amino | SRGDRPM |
| | acid sequence | |
| 44 | nucleotide sequence encoding AVT905 | tctaggggtgatcggcCtatg |
| | VP1's insert peptide | |
| 170 | AVT905 VP1's amino acid sequence | SAQSRGDRPM |
| | corresponding to position 586-588 of | |
| | wild-type AAV9 VP1 plus insert peptide | |
| 10 | AVT906 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALFHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQSAQLHKGIDIAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 45 | AVT906 VP1's insert peptide amino | LHKGIDI |
| | acid sequence | |
| 46 | nucleotide sequence encoding AVT906 | ctgcataaggggattgatatt |
| | VP1's insert peptide | |
| 171 | AVT906 VP1's amino acid sequence | SAQLHKGIDI |
| | corresponding to position: 586-588 of | |
| | wild-type AAV9 VP1 plus insert peptide | |
| 11 | AVT907 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEFAAKTAPGKKRPV |
| | acid sequence; italie: insert after wild- | EQSPQEPDSSAGIGKSGAQPAKKRENFGQTGDTESVPDPQPIGEPPAAPSGV |
| | type position 453; italic and underlined: | GSLTMASGGGAPVADNNEGADGVGSSSGNWHCDSQWLGDRVITTSTRTW |
| | insert after wild-type position 588) | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GHMNGRNNSGQNQQTLKFSVAGPSNMAVQGRNYIPGPSYRQQRVSTTVTQ |
| | | NNNSEFAWPGASSWALNGRNSLMNPGPAMASIIKEGEDRFFPLSGSLIFGK |
| | | QGTGRDNVDADKVMITNEEEIKTTNPVATESYGQVATNIIQGPGRGDQTTL |
| | | AQAQTGWVQNQGILPGMVWQDRDVYLQGPIWAKIPIITDGNFIIPSPLMGG |
| | | FGMKIIPPPQILIKNTPVPADPPTAFNKDKLNSFITQYSTGQVSVEIEWELQK |
| | | ENSKRWNPEIQYTSNYYKSNNVEFAVNTEGVYSEPRPIGTRYLTRNL |
| 47 | AVT907 VP1's insert peptide (after | HMNGRNN |
| | wild-type position 453) amino acid | |
| | sequence | |
| 48 | nucleotide sequence encoding AVT907 | catatgaatgggcgtaataat |
| | VP1's insert peptide (after wild-type: | |
| | position 453) | |
| 49 | AVT907 VP1's amino acid sequence | GPGRGDQTTL |
| | corresponding to position 586-588 of | |
| | wild-type AAV9 VP1, plus insert | |
| | peptide (after wild-type position 588) | |
| 50 | nucleotide sequence encoding AVT907 | ggccccggcagaggagaccagaccaccctg |
| | VP1's corresponding to position 586- | |
| | 588 of wild-type AAV9 VP1, and insert | |
| | after wild-type position 588 | |
| 12 | AVT908 VP1 amino acid sequence | MAADGYLPDWLEDNLSEGIREWWALKTGAPQPKANQQHQDNARGLVLP |
| | (underlined: corresponding to position | GYKYLGPGNGLDKGEPVNAADAAALFHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQSAQSRGDRPMAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 51 | AVT908 VP1's insert peptide amino | SRGDRPM |
| | acid sequence | |
| 52 | nucleotide sequence encoding AVT908 | tccagaggcgaccggcctatg |
| | VP1's insert peptide | |
| 172 | AVT908 VP1's amino acid sequence | SAQSRGDRPM |
| | corresponding to position 586-588 of | |
| | wild-type AAV9 VP1 plus insert peptide | |
| 13 | AVT909 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKERLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDQSQAVQRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNIIQSAQGVNGRSTAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 53 | AVT909 VP1's insert peptide amino | GVNGRST |
| | acid sequence | |
| 54 | nucleotide sequence encoding AVT909 | ggtgtgaatgggaggagtact |
| | VP1's insert peptide | |
| 173 | AVT909 VP1's amino acid sequence | SAQGVNGRST |
| | corresponding to position 586-588 of | |
| | wild-type AAV9 VP1 plus insert peptide | |
| 14 | AVT910 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAFFQERLKEDTSFGGNLGRAVFQAKKRLLEPLGLVEFAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSIRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSEYCLEYF |
| | | PSQMLRTGNNFQFSYEFENVPFHSSYAHSQSLDRLMNPLIDQYLYYLSKTIN |
| | | GSGQNQQTLKESVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEFIKTTNPVATESYGQVATNHQSAQGVNGRQVAQAQTG |
| | | WVQNQGILPGMVWQDRDVYLQGPIWAKIPHTDGNFHPSPLMGGEGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLIRNL |
| 55 | AVT910 VP1's insert peptide amino | GVNGRQV |
| | acid sequence | |
| 56 | nucleotide sequence encoding AVT910 | ggggttaatgggcggcaggtg |
| | VP1's insert peptide | |
| 174 | AVT910 VP1's amino acid sequence | SAQGVNGRQV |
| | corresponding to position 586-588 of | |
| | wild-type AAV9 VP1 plus insert peptide | |
| 15 | AVT911 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic: insert after wild- | EQSPQEPDSSAGIGKSGAQPAKKRLNFGQTGDTESVPDPQPIGEPPAAPSGV |
| | type position 453) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GQTYTHNKSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGK |
| | | QGTGRDNVDADKVMITNEEEIKTTNPVATESYGQVATNHQSAQAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 57 | AVT911 VP1's insert peptide amino | QTYTHNK |
| | acid sequence | |
| 58 | nucleotide sequence encoding AVT911 | cagacgtatacgcataataag |
| | VP1's insert peptide | |
| 16 | AVT912 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic: insert after wild- | EQSPQEPDSSAGIGKSGAQPAKKRLNFGQTGDTESVPDPQPIGEPPAAPSGV |
| | type position 453; italic and underlined; | GSLTMASGGGAPVADNNEGADGVGSSSGNWHCDSQWLGDRVITTSTRTW |
| | insert after wild-type position 588) | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVQRSSFYCLEYF |
| | | PSQMLRTGNNFQFSYEFENVPFHSSYAHSQSLDRLMNPLIDQYLYYLSKTIN |
| | | GQTYTHNKSGQNQQTLKFSVAGPSNMAVQGRNYIPGPSYRQQRVSTIVTQ |
| | | NNNSEFAWPGASSWALNGRNSLMNPGPAMASHKEGEDRFFPLSGSLIFGK |
| | | QGTGRDNVDADKVMITNEEEIKTTNPVATESYGQVATNHQSAQSRGDRPM |
| | | AQAQTGWVQNQGILPGMVWQDRDVYLQGPIWAKIPHTDGNFHPSPLMGG |
| | | FGMKHPPPQILIKNTPVPADPPTAFNKDKLNSFITQYSTGQVSVEIEWELQK |
| | | ENSKRWNPEIQYTSNYYKSNNVEFAVNTEGVYSEPRPIGTRYLTRNL |
| 59 | AVT912 VP1's insert peptide (after | QTYTHNK |
| | wild-type position 453) amino acid | |
| | sequence | |
| 60 | nucleotide sequence encoding AVT912 | cagacgtatacgcataataag |
| | VP1's insert after wild-type position 453 | |
| | peptide | |
| 61 | AVT912 VP1's insert peptide (after | SRGDRPM |
| | wild-type position 588) amino acid | |
| | sequence | |
| 62 | nucleotide sequence encoding AVT912 | tctaggggtgatcggcctatg |
| | VP1's insert peptide (after wild-type | |
| | position 588) | |
| 175 | AVT912 VP1's amino acid sequence | SAQSRGDRPM |
| | corresponding to position 586-588 of | |
| | wild-type AAV9 VP1 plus insert peptide | |
| | afterwards | |
| 17 | AVT913 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPEPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTINPVATESYGQVATNIIQENRRGDTNALAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 63 | AVT913 VP1's amino acid sequence | ENRRGDTNAL |
| | corresponding to position 586-588 of | |
| | wild-type AAV9 VP1 plus insert peptide | |
| 64 | nucleotide sequence encoding AVT913 | caggagaacaggagaggagacacgaacgctctggea |
| | VP1's amino acid sequence | |
| | corresponding to position 586-588 of | |
| | wild-type AAV9 VP1 plus insert peptide | |
| 18 | AVT914 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLILNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQNNSRGDHNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 65 | AVT914 VP1's amino acid sequence | NNSRGDHNSL |
| | corresponding to position 586-588 of | |
| | wild-type AAV9 VP1 plus insert peptide | |
| 66 | nucleotide sequence encoding AVT914 | cagaataactcgagaggagaccataactcgctggca |
| | VP1's amino acid sequence | |
| | corresponding to position 586-588 of | |
| | wild-type AAV9 VP1 plus insert peptide | |
| 19 | AVT915 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFIISSYALISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQNNSRGDYNSLAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 67 | AVT915 VP1's amino acid sequence | NNSRGDYNSL |
| | corresponding to position 586-588 of | |
| | wild-type AAV9 VP1 plus insert peptide | |
| 68 | nucleotide sequence encoding AVT915 | cagaataacagtagaggagactataacagtctggca |
| | VP1's amino acid sequence | |
| | corresponding to position 586-588 of | |
| | wild-type AAV9 VP1 plus insert peptide | |
| 20 | AVT916 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSIRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQNNLRGDHNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 69 | AVT916 VP1's amino acid sequence | NNLRGDHNSL |
| | corresponding to position 586-588 of | |
| | wild-type AAV9 VP1 plus insert peptide | |
| 70 | nucleotide sequence encoding AVT916 | cagaataacctgagaggagaccataactctctggca |
| | VP1's amino acid sequence | |
| | corresponding to position 586-588 of | |
| | wild-type AAV9 VP1 plus insert peptide | |
| 21 | AVT917 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQNNARGDHNSLAQAQTG |
| | | WVQNQGILPGMVWQDRDVYLQGPIWAKIPIITDGNFIIPSPLMGGFGMKIIP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 71 | AVT917 VP1's amino acid sequence: | NNARGDHNSL |
| | corresponding to position 586-588 of | |
| | wild-type AAV9 VP1 plus insert peptide | |
| 72 | nucleotide sequence encoding AVT917 | cagaataacgctagaggagaccataacagtctggca |
| | VP1's amino acid sequence: | |
| | corresponding to position 586-588 of | |
| | wild-type AAV9 VP1 plus insert peptide | |
| 22 | AVT918 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNEKLFNIQVKEVTDNNGVKTIANNLTSTVQVFTDSD |
| | | YQLPYVLQSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSQSLIFGKQGTGRDN |
| | | VDADKVMITNEEEIKTINPVATESYGQVATNHQGNIRGDHNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 73 | AVT918 VP1's amino acid sequence | GNIRGDHNTL |
| | corresponding to position 586-588 of | |
| | wild-type AAV9 VP1 plus insert peptide | |
| 74 | nucleotide sequence encoding AVT918 | cagggtaacattagaggagaccataacactctggca |
| | VP1's amino acid sequence | |
| | corresponding to position 586-588 of | |
| | wild-type AAV9 VP1 plus insert peptide | |
| 23 | AVT919 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFEPLSGSLIFGKQGTGRDN |
| | | VDADKVMITNEEEIKTINPVATESYGQVATNHQMNVRGDTNKLAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 75 | AVT919 VP1's amino acid sequence | MNVRGDTNKL |
| | corresponding to position 586-588 of | |
| | wild-type AAV9 VP1 plus insert peptide | |
| 76 | nucleotide sequence encoding AVT919 | cagatgaacgtgagaggagacactaacaagctggca |
| | VP1's amino acid sequence | |
| | corresponding to position 586-588 of | |
| | wild-type AAV9 VP1 plus insert peptide | |
| 24 | AVT920 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVERAAKTAPGKKRPV |
| | acid sequence; italic and underlined: | FQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDREFPLSGSLIFGKQGTGRDN |
| | | VDADKVMITNEEEIKTINPVATESYGQVATNHQTNMRGDHNALAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 77 | AVT920 VP1's amino acid sequence | TNMRGDHNAL |
| | corresponding to position 586-588 of | |
| | wild-type AAV9 VP1 plus insert peptide | |
| 78 | nucleotide sequence encoding AVT920 | cagacgaacatgagaggagaccataacgcgctggca |
| | VP1's amino acid sequence | |
| | corresponding to position 586-588 of | |
| | wild-type AAVY VP1 plus insert peptide | |
| 25 | AV9ML001 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVETDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNFFRIKTTNPVATESYGQVATNHQNNNRGDNNYLAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLIRNL |
| 79 | AV9ML001 VP1's amino acid sequence | NNNRGDNNYL |
| | corresponding to position 586-588 of | |
| | wild-type AAV9 VP1 plus insert peptide | |
| 100 | nucleotide sequence encoding | aacaacaacagaggegacaacaactacctc |
| | AV9ML001 VP1's amino acid sequence | |
| | corresponding to position 586-588 of | |
| | wild-type AAV9 VP1 plus insert peptide | |
| 26 | AV9ML002 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTINPVATESYGQVAINIIQNNNRGDHNTLAQAQTG |
| | | WVQNQGILPGMVWQDRDVYLQGPIWAKIPIITDGNFIIPSPLMGGFGMKIIP |
| | | PPQILIKNTPVPADPPTAFNKDKLNSFITQYSTGQVSVEIEWELQKENSKRW |
| | | NPEIQYTSNYYKSNNVEFAVNTEQVYSEPRPIGTRYLTRNL |
| 80 | AV9ML002 VP1's amino acid sequence | NNNRGDHNTL |
| | corresponding to position 586-588 of | |
| | wild-type AAV9 VP1 plus insert peptide | |
| 101 | nucleotide sequence encoding | aacaacaacagaggcgaccacaacaccctc |
| | AV9ML002 VP1's amino acid sequence | |
| | corresponding to position 586-588 of | |
| | wild-type AAV9 VP1 plus insert peptide | |
| 27 | AV9ML004 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEFFIKTTNPVATESYGQVATNHQNNERGDRNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLIRNL |
| 81 | AV9ML004 VP1's amino acid sequence | NNERGDRNSL |
| | corresponding to position 586-588 of | |
| | wild-type AAV9 VP1 plus insert peptide | |
| 102 | nucleotide sequence encoding | aacaacgaaagaggcgacagaaacagcctc |
| | AV9ML004 VP1's amino acid sequence | |
| | corresponding to position 586-588 of | |
| | wild-type AAV9 VP1 plus insert peptide | |
| 28 | AV9ML005 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQFRLKEDTSFGGNLGRAVFQAKKRLLEPLGLVERAAKTAPGKKRPV |
| | acid sequence; italic and underlined: | FQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLISTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQNNSRGDHNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| $2 | AV9ML005 VP1's amino acid sequence | NNSRGDIINTL |
| | corresponding to position 586-588 of | |
| | wild-type AAV9 VP1 plus insert peptide | |
| 103 | nucleotide sequence encoding | aacaacagcagaggegaccacaacaccctc |
| | AV9ML005 VP1's amino acid sequence | |
| | corresponding to position 586-588 of | |
| | wild-type AAV9 VP1 plus insert peptide | |
| 29 | AV9ML007 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVETDSD |
| | | YQLPYVLGSAHEGCLPPFPADVEMIPQYGYLILNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQENKRGDHNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 83 | AV9ML007 VP1's amino acid sequence | ENKRGDHNSL |
| | corresponding to position 586-588 of | |
| | wild-type AAV9 VP1 plus insert peptide | |
| 104 | nucleotide sequence encoding | gaaaacaaaagaggcgaccacaacagcctc |
| | AV9ML007 VP1's amino acid sequence | |
| | corresponding to position 586-588 of | |
| | wild-type AAV9 VP1 plus insert peptide | |
| 30 | AV9ML012 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVERAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVIDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTIVTQNNNSEFA |
| | | WPGASSWALNGRNSLMNPGPAMASHKEGEDRFFPLSGSLIEGKQGTGRDN |
| | | VDADKVMITNEEEIKTTNPVATESYGQVATNHQINSRGDHNVLAQAQTGW |
| | | VQNQGILPGMYWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 84 | AV9ML012 VP1's amino acid sequence: | INSRGDHNVL |
| | corresponding to position 586-588 of | |
| | wild-type AAV9 VP1 plus insert peptide | |
| 105 | nucleotide sequence encoding | atcaacagcagaggcgaccacaacgtcctc |
| | AV9ML012 VP1's amino acid sequence | |
| | corresponding to position 586-588 of | |
| | wild-type AAV9 VP1 plus insert peptide | |
| 31 | AV9ML014 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNEGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHFSPRDW |
| | | QRLINNNWGFRPKRLNEKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQNNNRGDYNSLAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 85 | AV9ML014 VP1's amino acid sequence | NNNRGDYNSL |
| | corresponding to position 586-588 of | |
| | wild-type AAV9 VP1 plus insert peptide | |
| 106 | nucleotide sequence encoding | aacaacaacagaggcgactacaacagcctc |
| | AV9ML014 VP1's amino acid sequence | |
| | corresponding to position 586-588 of | |
| | wild-type AAV9 VP1 plus insert peptide | |
| 32 | AV9ML016 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLIMASGGGAPVADNNEGADGVGSSSGNWIICDSQWLGDRVITTSTRTW |
| | | ALPTYNNIILYKQISNSTSGGSSNDNAYFGYSTPWGYFDFNRFIICHIFSPRDW |
| | | QRLINNNWGFRPKRLNFKLENIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEREIKTTNPVATESYGQVATNHQSAQRGDKLSIAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 86 | AV9ML016 VP1's insert peptide amino | RGDKLSI |
| | acid sequence | |
| 107 | nucleotide sequence encoding | agaggcgacaaactcagcatc |
| | AV9ML016 VP1's amino acid sequence | |
| | corresponding to position 586-588 of | |
| | wild-type AAV9 VP1 plus insert peptide | |
| 176 | AV9ML016 VP1's amino acid sequence | SAQRGDKLSI |
| | corresponding to position 586-588 of | |
| | wild-type AAV9 VP1 plus insert peptide | |
| 33 | AV9ML018 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLQRAVFQAKKRLLEPLGLVEEAAKTAPGKKRPV |
| | acid sequence; italic and underlined; | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPEHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNIIQSAQLNTNNSFTKAQAQTG |
| | | WVQNQGILPGMVWQDRDVYLQGPIWAKIPIIIDGNFIIPSPLMGGFGMKIIP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLIRNL |
| 87 | AV9ML018 VP1's insert peptide amino | LNTNNSFTK |
| | acid sequence | |
| 108 | nucleotide sequence encoding | ctcaacaccaacaacagcttcaccaaa |
| | AV9ML018 VP1's amino acid sequence | |
| | corresponding to position 586-588 of | |
| | wild-type AAV9 VP1 plus insert peptide | |
| 177 | AV9ML018 VP1's amino acid sequence | SAQLNTNNSFTK |
| | corresponding to position 586-588 of | |
| | wild-type AAV9 VP1 plus insert peptide | |
| 115 | AV9ML025 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italie and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNEKLENIQVKENTDNNGVKTIANNLISTVQVETDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHIKEGEDRFFPLSGSLIFGKQGTGRDN |
| | | VDADKVMITNEEEIKTINPVATESYGQVATNIIQSNSRGDHNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 116 | AV9ML025 VP1's amino acid sequence | SNSRGDHNTL |
| | corresponding to position 586-588 of | |
| | wild-type AAV9 VP1 plus insert peptide | |
| | insert peptide | |
| 117 | AV9ML026 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italie and underlined: | EQSPQEPDSSAGIGKSGAQPAKKRLNFGQTGDIESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNIILYKQISNSTSGGSSNDNAYFGYSTPWGYFDFNRFHICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | QSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVAINHQSNSRGDHNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 118 | AV9ML026 VP1's amino acid sequence | SNSRGDIINSL |
| | corresponding to position 586-588 of | |
| | wild-type AAV9 VP1 plus insert peptide | |
| | insert peptide | |
| 119 | AV9ML027 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNEGQTGDIESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLENIQVKEVTDNNGVKTIANNLTSTVQVFTDSD |
| | | YQLPYVLGSAHEGCLPPFPADVFMIPQYGYLILNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQENRRGDHASLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | ETQYTSNYYKSNNVEFAVNTEGVYSEPRPIGTRYLTRNL |
| 120 | AV9ML027 VP1's amino acid sequence | ENRRGDHNSL |
| | corresponding to position 586-588 of | |
| | wild-type AAV9 VP1 plus insert peptide | |
| | insert peptide | |
| 121 | AV9ML028 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEFEIKTTNPVATESYGQVATNHQGNSRGDYNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 122 | AV9ML028 VP1's amino acid sequence | GNSRGDYNSL |
| | corresponding to position 586-588 of | |
| | wild-type AAV9 VP1 plus insert peptide | |
| | insert peptide | |
| 123 | AV9ML029 VP1 amino acid sequence | MAADGYLPDWLEDNLSEGIREWWALKPGAPQPKANQQHIQDNARGLVLP |
| | (underlined: corresponding to position | GYKYLGPGNGLDKGEPVNAADAAALEIIDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKESVAGPSNMAVQGRNYIPGPSYRQQRVSTTVTQNNNSEFA |
| | | WPGASSWALNGRNSLMNPGPAMASHKEGEDRFEPLSGSLIFGKQGTGRDN |
| | | VDADKVMITNEEEIKTTNPVATESYGQVATNHQNNSRGDRNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 124 | AV9ML029 VP1's amino acid sequence | NNSRGDRNSL |
| | corresponding to position: 586-588 of | |
| | wild-type AAV9 VP1 plus insert peptide: | |
| | insert peptide | |
| 125 | AV9ML030 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPEPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGIGRDN |
| | | VDADKVMITNEEEIKTINPVATESYGQVATNHQMNVRGDTNSLAQAQTG |
| | | WVQNQGILPGMVWQDRDVYLQGPIWAKIPIITDGNFTIPSPLMGGFGMKHIP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 126 | AV9ML030 VP1's amino acid sequence | MNVRGDINSL |
| | corresponding to position 586-588 of | |
| | wild-type AAV9 VP1 plus insert peptide | |
| | insert peptide | |
| 127 | AV9ML031 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVEQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQSNARGDHNSLAQAQTGW |
| | | VQNQGILPGMVWQDRDVYLQGPIWAKIPIITDGNFHIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 128 | AV9ML031 VP1's amino acid sequence | SNARGDIINSL |
| | corresponding to position 586-588 of | |
| | wild-type AAV9 VP1 plus insert peptide | |
| | insert peptide | |
| 129 | AV9ML032 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVERAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLIMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVEMIPQYGYLILNDGSQAVGRSSFYCLEYF |
| | | PSQMLRTUNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQGNSRGDHNTLAQAQTGW |
| | | VQNQGILPGMVWQDRDVYLQGPIWAKIPHIDGNFHPSPLMGGFGMKHPPP |
| | | QILIKNIPVPADPPTAFNKDKLNSFITQYSTGQVSVEIEWELQKENSKRWNP |
| | | EIQYTSNYYKSNNVEFAVNTEGVYSEPRPIGTRYLTRNL |
| 130 | AV9ML032 VP1's amino acid sequence | GNSRGDHNTL |
| | corresponding to position 586-588 of | |
| | wild-type AAV9 VP1 plus insert peptide | |
| | insert peptide | |
| 131 | AV9ML033 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQNNNRGDHNALAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 132 | AV9ML033 VP1's amino acid sequence | NNNRGDHNAL |
| | corresponding to position 586-588 of | |
| | wild-type AAV9 VP1 plus insert peptide | |
| | insert peptide | |
| 133 | AV9ML034 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQNNLRGDANSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 134 | AV9ML034 VP1's amino acid sequence | NNLRGDRNSL |
| | corresponding to position 586-588 of | |
| | wild-type AAV9 VP1 plus insert peptide | |
| | insert peptide | |
| 135 | AVYML035 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQDNRRGDTNALAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 136 | AV9ML035 VP1's amino acid sequence | DNRRGDTNAL |
| | corresponding to position 586-588 of | |
| | wild-type AAV9 VP1 plus insert peptide | |
| | insert peptide | |
| 137 | AV9ML036 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVEMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTINPVATESYGQVATNIIQENERGDHNSLAQAQTGW |
| | | VQNQGILPGMVWQDRDVYLQGPIWAKIPIITDGNFIIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLIRNL |
| 138 | AV9ML036 VP1's amino acid sequence | ENERGDHNSL |
| | corresponding to position 586-588 of | |
| | wild-type AAV9 VP1 plus insert peptide | |
| | insert peptide | |
| 139 | AV9ML037 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTINPVATESYGQVATNIIQNNARGDTNALAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 140 | AV9ML037 VP1's amino acid sequence | NNARGDINAL |
| | corresponding to position 586-588 of | |
| | wild-type AAV9 VP1 plus insert peptide | |
| | insert peptide | |
| 141 | AV9ML038 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNIILYKQISNSTSGGSSNDNAYFGYSTPWGYFDFNRFHICIIFSPRDW |
| | | QRLINNNWGFRPKRLNEKLFNIQVKEVTDNNGVKTIANNLISTVQVETDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTINPVATESYGQVATNHQSNLRGDHASLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 142 | AV9ML038 VP1's amino acid sequence | SNLRGDIINSL |
| | corresponding to position 586-588 of | |
| | wild-type AAV9 VP1 plus insert peptide | |
| | insert peptide | |
| 143 | AV9ML039 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined; | EQSPQEPDSSAGIGKSGAQPAKKRLNEGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNEKLFNIQVKENTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHIKEGEDRFFPLSGSLIFGKQGTGRDN |
| | | VDADKVMITNEEEIKTINPVATESYGQVATNIIQMSVRGDTNKLAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 144 | AV9ML039 VP1's amino acid sequence | MSVRGDINKL |
| | corresponding to position 586-588 of | |
| | wild-type AAV9 VP1 plus insert peptide | |
| | insert peptide | |
| 145 | AV9ML040 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined; | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSIRTW |
| | | ALPTYNNIILYKQISNSTSGGSSNDNAYFGYSTPWGYFDFNRFHICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQMNVRGDTNKPAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 146 | AV9ML040 VP1's amino acid sequence | MNVRGDTNKP |
| | corresponding to position 586-588 of | |
| | wild-type AAV9 VP1 plus insert peptide | |
| | insert peptide | |
| 147 | AV9ML041 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGY |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVETDSD |
| | | YQLPYVLGSAHEGCLPPEPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNIIQNNLRGDTNALAQAQTGW |
| | | VQNQGILPGMVWQDRDVYLQGPIWAKIPHITDGNFIIPSPLMGGFGMKIIPPP |
| | | QILIKNTPVPADPPTAFNKDKLNSFITQYSTGQVSVELEWELQKENSKRWNP |
| | | EIQYTSNYYKSNNVEFAVNTEGVYSEPRPIGTRYLTRNL |
| 148 | AV9ML041 VP1's amino acid sequence | NNLRGDTNAL |
| | corresponding to position 586-588 of | |
| | wild-type AAV9 VP1 plus insert peptide | |
| | insert peptide | |
| 149 | AV9ML042 VP1 amino acid sequence | MAADGYLPDWLEDNLSEGIREWWALKPGAPQPKANQQHQDNARGLVLP |
| | (underlined; corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVETDSD |
| | | YQLPYVLGSAHEGCLPPFPADVFMIPQYGYLTLNDGSQAVGRSSFYCLEYF |
| | | PSQMLRTUNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDREFPLSGSLIFGKQGTGRDN |
| | | VDADKVMITNEEEIKTTNPVATESYGQVATNHQINSRGDYNVLAQAQTGW |
| | | VQNQGILPGMVWQDRDVYLQGPIWAKIPHTDGNEHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 150 | AV9ML042 VP1's amino acid sequence | INSRGDYNVL |
| | corresponding to position 586-588 of | |
| | wild-type AAV9 VP1 plus insert peptide | |
| | insert peptide | |
| 151 | AV9ML043 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | FQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNIILYKQISNSTSGGSSNDNAYFGYSIPWGYFDFNRFIICIIFSPRDW |
| | | QRLINNNWGFRPKRLNFKLFNIQVKEVTDNNGVKTIANNLTSTVQVFTDSD |
| | | YQLPYVLGSAIIEGCLPPFPADVEMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGIGRDN |
| | | VDADKVMITNEEEIKTTNPVATESYGQVATNHQKNRRGDTNALAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTFGVYSEPRPIGTRYLTRNL |
| 152 | AV9ML043 VP1's amino acid sequence | KNRRGDTNAL |
| | corresponding to position 586-588 of | |
| | wild-type AAV9 VP1 plus insert peptide | |
| | insert peptide | |
| 153 | AV9ML044 VP1 amino acid sequence: | MAADGYLPDWLEDNLSEGIREWWALKPGAPQPKANQQIIQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDIESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQENERGDRNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 154 | AV9ML044 VP1's amino acid sequence | ENERGDRNSL |
| | corresponding to position 586-588 of | |
| | wild-type AAV9 VP1 plus insert peptide | |
| | insert peptide | |
| 155 | AV9ML045 VP1 amino acid sequence | MAADGYLPDWLEDNLSEGIREWWALKPGAPQPKANQQUIQDNARGLVLP |
| | (underlined: corresponding to position | GYKYLGPGNGLDKGEPVNAADAAALEIIDKAYDQQLKAGDNPYLKYNIIA |
| | 586-588 of wild-type AAV9 VP1 amino | DAEFQERLKEDTSFGGNLGRAVFQAKKRLLEPLGLVEEAAKTAPGKKRPV |
| | acid sequence: italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKTTNPVATESYGQVATNHQMNARGDTNKLAQAQTG |
| | | WVQNQGILPGMVWQDRDVYLQGPIWAKIPHTDGNFHPSPLMGGFGMKHP |
| | | PPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| 156 | AV9ML045 VP1's amino acid sequence | MNARGDTNKL |
| | corresponding to position 586-588 of | |
| | wild-type AAV9 VP1 plus insert peptide | |
| | insert peptide | |
| 157 | AV9ML046 VP1 antino acid sequence | MAADGYLPDWLEDNLSEGIREWWALKPGAPQPKANQQHIQDNARGLVLP |
| | (underlined: corresponding to position | GYKYLGPGNGLDKGEPVNAADAAALEIIDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKESVAGPSNMAVQGRNYIPGPSYRQQRVSTTVTQNNNSEFA |
| | | WPGASSWALNGRNSLMNPGPAMASHKEGEDRFEPLSGSLIFGKQGTGRDN |
| | | VDADKVMITNEEEIKTTNPVATESYGQVATNHQGNIRGDRNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 158 | AV9ML046 VP1's amino acid sequence | GNIRGDRNTL |
| | corresponding to position: 586-588 of | |
| | wild-type AAV9 VP1 plus insert peptide | |
| | insert peptide | |
| 159 | AV9ML047 VP1 amino acid sequence | MAADGYLPDWLEDNLSEGIREWWALKPGAPQPKANQQIIQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGIGRDN |
| | | VDADKVMITNEEEIKTINPVATESYGQVATNHQGNIRGDHNALAQAQTGW |
| | | VQNQGILPGMVWQDRDVYLQGPIWAKIPIITDGNFHIPSPLMGGFGMKIIPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 160 | AV9ML047 VP1's amino acid sequence | GNIRGDHNAL |
| | corresponding to position 586-588 of | |
| | wild-type AAV9 VP1 plus insert peptide | |
| | insert peptide | |
| 161 | AV9ML048 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSEGGNLGRAVEQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHESPRDW |
| | | QRLINNNWGFRPKRLNFKLFNIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDREFPLSGSLIFGKQGTGRDN |
| | | VDADKVMITNEEEIKTTNPVATESYGQVATNHQNNRRGDHNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 162 | AV9ML048 VP1's amino acid sequence | NNRRGDHNSL |
| | corresponding to position 586-588 of | |
| | wild-type AAV9 VP1 plus insert peptide | |
| | insert peptide | |
| 163 | AV9ML049 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined: | EQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYFGYSTPWGYFDFNRFHCHFSPRDW |
| | | QRLINNNWGFRPKRLNFKLFNIQVKEVTDNNGVKTIANNLTSTVQVFTDSD |
| | | YQLPYVLGSAIIEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASIIKEGEDRFFPLSGSLIFGKQGTGRDN |
| | | VDADKVMITNEEEIKTTNPVATESYGQVATNHQTNIRGDYNSLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 164 | AV9ML049 VP1's amino acid sequence | TNIRGDYNSL |
| | corresponding to position 586-588 of | |
| | wild-type AAV9 VP1 plus insert peptide | |
| | insert peptide | |
| 165 | AV9ML050 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVERAAKTAPGKKRPV |
| | acid sequence; italic and underlined: | FQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWHCDSQWLGDRVITTSTRTW |
| | | ALPTYNNHLYKQISNSTSGGSSNDNAYEGYSTPWGYFDFNRFHCHFSPRDW |
| | | QRLINNNWGFRPKRLNFKLENIQVKEVTDNNGVKTIANNLTSTVQVFTDSD |
| | | YQLPYVLGSAHEGCLPPFPADVFMIPQYGYLTLNDGSQAVGRSSFYCLEYF |
| | | PSQMLRTGNNFQFSYEFENVPFIISSYAIISQSLDRLMNPLIDQYLYYLSKTIN |
| | | GSGQNQQTLKESVAGPSNMAVQGRNYIPGPSYRQQRVSTTVTQNNNSEFA |
| | | WPGASSWALNGRNSLMNPGPAMASHKEGEDRFFPLSGSLIFGKQGTGRDN |
| | | VDADKVMITNEEEIKITNPVATESYGQVATNHQRNLRGDHNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIEWELQKENSKRWNP |
| | | EIQYTSNYYKSNNVEFAVNTEGVYSEPRPIGTRYLTRNL |
| 166 | AV9ML050 VP1's amino acid sequence | RNLRGDHNTL |
| | corresponding to position 586-588 of | |
| | wild-type AAV9 VP1 plus insert peptide | |
| | insert peptide | |
| 167 | AV9ML051 VP1 amino acid sequence | MAADGYLPDWLEDNLSEGIREWWALKPGAPQPKANQQHQDNARGLVLP |
| | (underlined: corresponding to position | GYKYLGPGNGLDKGEPVNAADAAALEHDKAYDQQLKAGDNPYLKYNHA |
| | 586-588 of wild-type AAV9 VP1 amino | DAEFQERLKEDTSFGGNLGRAVFQAKKRLLEPLGLVEEAAKTAPGKKRPV |
| | acid sequence; italic and underlined; | FQSPQEPDSSAGIGKSGAQPAKKRLNFGQTGDTESVPDPQPIGEPPAAPSGV |
| | insert after wild-type position 588) | GSLTMASGGGAPVADNNEGADGVGSSSGNWIICDSQWLGDRVITTSTRTW |
| | | ALPTYNNIILYKQISNSTSGGSSNDNAYFGYSTPWGYFDFNRFIICIIFSPRDW |
| | | QRLINNNWGFRPKRLNFKLFNIQVKEVTDNNGVKTIANNLTSTVQVETDSD |
| | | YQLPYVLGSAHEGCLPPFPADVFMIPQYGYLTLNDGSQAVGRSSFYCLEYF |
| | | PSQMLRTGNNFQFSYEFENVPFHSSYAHSQSLDRLMNPLIDQYLYYLSKTIN |
| | | GSGQNQQTLKFSVAGPSNMAVQGRNYIPGPSYRQQRVSTTVTQNNNSEFA |
| | | WPGASSWALNGRNSLMNPGPAMASHKEGEDRFFPLSGSLIFGKQGTGRIN |
| | | VDADKVMITNEEEIKTTNPVATESYGQVATNHQCNSRGDHNTLAQAQTGW |
| | | VQNQGILPGMVWQDRDVYLQGPIWAKIPHTDGNFHPSPLMGGFGMKHPPP |
| | | QILIKNTPVPADPPTAFNKDKLNSFITQYSTGQVSVEIFWELQKENSKRWNP |
| | | EIQYTSNYYKSNNVEFAVNTEGVYSEPRPIGTRYLTRNL |
| 168 | AV9ML051 VP1's amino acid sequence | CNSRGDHNTL |
| | corresponding to position 586-588 of | |
| | wild-type AAV9 VP1 plus insert peptide | |
| | insert peptide | |
| 88 | Amino acid sequence illustrating the | GXXXXXXXS |
| | insertion of a 7-mer peptide after | |
| | position 453 of wild-type AAV9 VP 1 to | |
| | generate Library #1 (X stands for any | |
| | amino acid residue) | |
| 89 | Amino acid sequence illustrating the | QXXXXXXXA |
| | insertion of a 7-mer peptide after | |
| | position 588 of wild-type AAV9 VP 1 to | |
| | generate Library #2 (X stands for any | |
| | amino acid residue) | |
| 90 | Amino acid sequence illustrating the | QXXXXXXXXXA |
| | insertion of a 9-mer peptide after | |
| | position 588 of wild-type AAV9 VP 1 to | |
| | generate Library #3 (X stands for any | |
| | amino acid residue) | |
| 91 | Amino acid sequence illustrating the | QXNXRGDXNXLA |
| | substitution of positions 586-588 of | |
| | wild-type AAV9 VP1 and insertion of a | |
| | 7-mer peptide after position 588 to | |
| | generate Library #4 (X stands for any | |
| | amino acid residue) | |
| 92 | Cap9-453-7AA-PF primer nucleotide | ttcacggactcagactatcagctccc |
| | sequence | |
| 93 | Cap9-453-7AA-PR primer nucleotide | ttttagcgtttgttgattctgtccagamnnmnnmnnmnnmnnmnnmnnaccgttaatagtctttgagag |
| | sequence (N stands for A, G, T or C; M | |
| | stands for A or C) | |
| 94 | Cap9-588-7AA-PF primer nucleotide | taatagcttgatgaatcctggacctgcc |
| | sequence | |
| 95 | Cap9-588-7AA-PR primer nucleotide | ttttgaacccagccggtctgcgcctgtgcmnnmnnmnnmnnmnnmnnmnnttgggcactctggtggtttgtg |
| | sequence (N stands for A, G, T or C; M | |
| | stands for A or C) | |
| 96 | Cap9-588-9AA-PF primer nucleotide | taatagcttgatgaatcctggacctgcc |
| | sequence | |
| 97 | Cap9-588-9AA-PR primer nucleotide | ttttgaacccagccggtctgcgcctgtgcmnnmnnmnnmnnmnnmnnmnnmnnmnnttgggcactctggt |
| | sequence (N stands for A, G, T or C; M | ggtttgtg |
| | stands for A or C) | |
| 98 | Cap9-R4A-PF primer nucleotide | taatagcttgatgaatcctggacctgcc |
| | sequence | |
| 99 | Cap9-R4A-PR primer nucleotide | ttttgaacccagccggtctgcgcctgtgccagmnngttmnngtctcctctmnngttmnnctggtggtttgtg |
| | sequence (N stands for A, G, T or C; M | |
| | stands for A or C) | |

### EXAMPLES

Certain embodiments provided herein are illustrated by the following non-limiting examples, which describe the identification and testing of a number of variant AAV9 capsid proteins with improved tissue transduction.

### Example 1: Engineered AAV9 Variants for Enhanced Tissue Targeting

### Introduction

Directed AAV capsid evolution provides rapid selection of novel capsids with improved tissue transduction. AAV9-based capsid engineering was designed in this study by peptide insertion and substitution (at variable region IV and VIII) library screening in mouse and Cynomolgus macaque using synthetic promoter driven viral mRNA recovery from specific tissues.

### AAV Library Design

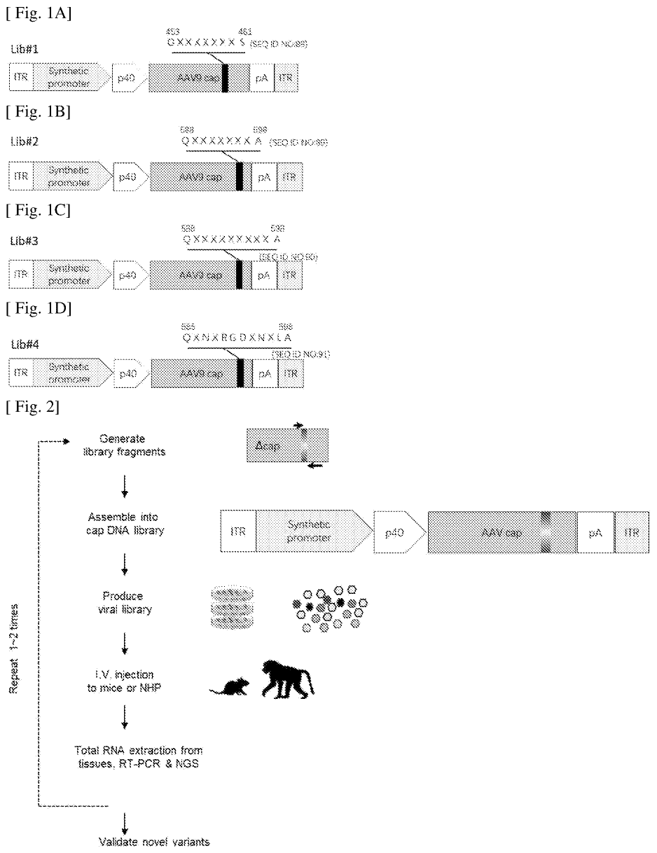

Peptide display random libraries (Lib #1-4) were designed by inserting 7 amino acids (7mer), or 9mer after Q588 (VP1 position of AAV9) in the Variable Region (VR) VIII or after G453 in the VR IV of AAV9 (FIGS. 1A-1D). The insert DNA library #1-4 were generated by PCR using the following primers.

TABLE 2
PCR Primers for Insert DNA Library#1-4.

| Library | primer name | Sequence |
|---|---|---|
| Lib#1 | Cap9-453-7AA-PF | TTCACGGACTCAGACTATCAGCTCCC |
| | (SEQ ID NO: 92) | |
| | Cap9-453-7AA-PR | TTTTAGCGTTTGTTGATTCTGTCCAGAMNNMNNMNNMNNMNNMNNMNNAC |
| | (SEQ ID NO: 93) | CGTTAATAGTCTTTGAGAG |
| Lib#2 | Cap9-588-7AA-PF | TAATAGCTTGATGAATCCTGGACCTGCC |
| | (SEQ (D NO: 94) | |
| | Cap9-588-7AA-PR | TTTTGAACCCAGCCGGTCTGCGCCTGTGCMNNMNNMNNMNNMNNMNNMNN |
| | (SEQ ID NO: 95) | TTGGGCACTCTGGTGGTTTGTG |
| Lib#3 | Cap9-588-9AA-PF | TAATAGCTTGATGAATCCTGGACCTGCC |
| | (SEQ ID NO: 96) | |
| | Cap9-588-9AA-PR | TTTTGAACCCAGCCGGTCTGCGCCTGTGCMNNMNNMNNMNNMNNMNNMNN |
| | (SEQ ID NO: 97) | MNNMNNTTGGGCACTCTGGTGGTTTGTG |
| Lib#4 | Cap9-R4A-PF | TAATAGCTTGATGAATCCTGGACCTGCC |
| | (SEQ ID NO: 98) | |
| | Cap9-R4A-PR | TTTTGAACCCAGCCGGTCTGCGCCTGTGCcagMNNgttMNNgtctcctct |
| | (SEQ ID NO: 99) | mnngttMNNCTGGTGGTTTGTG |

### AAV Library Screening Workflow

The DNA fragment libraries were generated by PCR with AAV9 library backbone DNA template. The purified PCR products were assembled into the inverted terminal repeat (ITR)-containing library backbone with synthetic promoter, p41, AAV9 (Cap9) VP1 and bovine growth hormone (bGH) poly A. The p41 promoter was used to drive capsid gene expression for AAV library production only in the presence of Ad5 helper gene products. The synthetic promoter was aimed to recover Cap9 mRNA expressed in specific tissues. The assembled library plasmid DNA was transformed to the competent cells to amplify. The amplified cap9 library plasmids were used for AAV library production by co-transfecting pHelper and pRep2-AAP (SEQ ID NO. 1) in HEK293 cells. The purified AAV viral libraries (Lib #1, Lib #2, Lib #3) were intravenously injected to 8-week-old mice (C57BL/6J, DBA/2J-mdx, BALB/cJ) at 4e12 vg/mouse. Twenty-one days after injection, the muscle tissue (triceps, quadriceps, gastrocnemius, abdominal, tibialis anterior, diaphragm, heart) and liver were collected for total RNA isolation. By using Cap9 specific reverse transcription primer, the viral mRNA was converted to cDNA for specific PCR amplification and the second round of library generation.

For screening in non-human primate (NHP), viral libraries (Lib #2, Lib #4) were intravenously injected to Cynomolgus macaque and the tissues were harvested 21 days after injection. The total RNA was isolated and treated with Dnase I. The cap9 specific mRNA was reverse transcribed and amplified by PCR (RT-PCR) for next-generation sequencing (NGS). The enriched candidates were selected by customized enrichment score analysis. A total of 810 candidates including variants predicted by artificial intelligence modeling were synthesized for the second round of screening. Meanwhile, 27 top variants were individually packaged for in vitro and in vivo characterization. These top variants were selected by NSG enrichment score. All variants were enriched in muscle, while AVT905 was enriched in liver. The nucleotide sequence encoding the peptide insert of one variant, AVT905, was codon-optimized; the corresponding variant was named AVT908 and was also characterized (see Table 3, below, wherein the lower case letters in the nucleotide sequence of AVT908 represent codon optimization). AVT908 and AVT905 share the same amino acid sequence, and thus AVT908 was also expected to be enriched in liver.

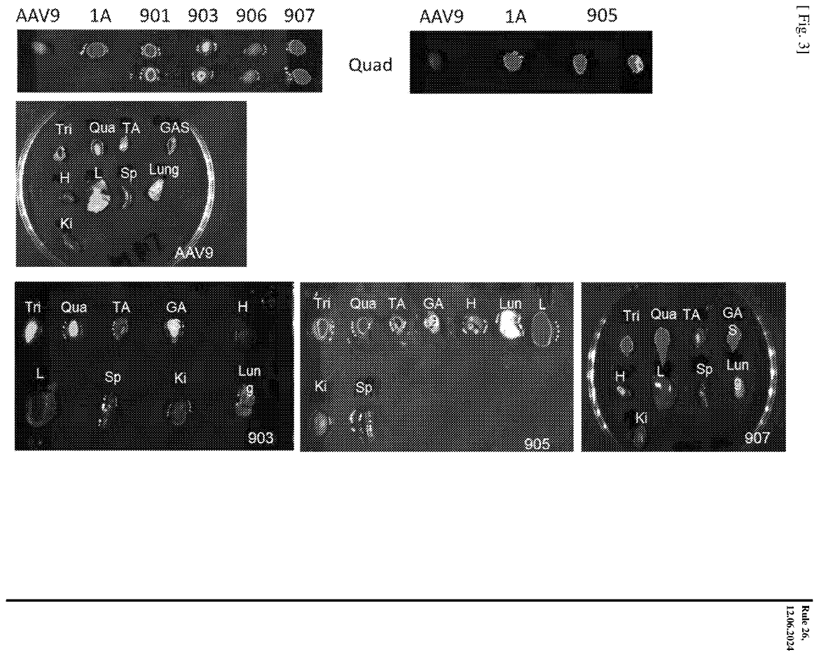

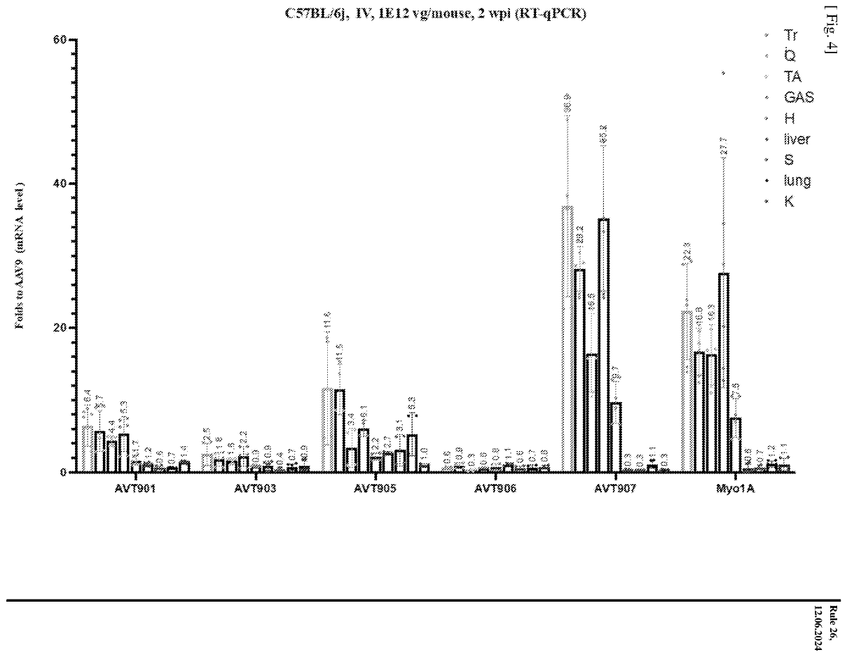

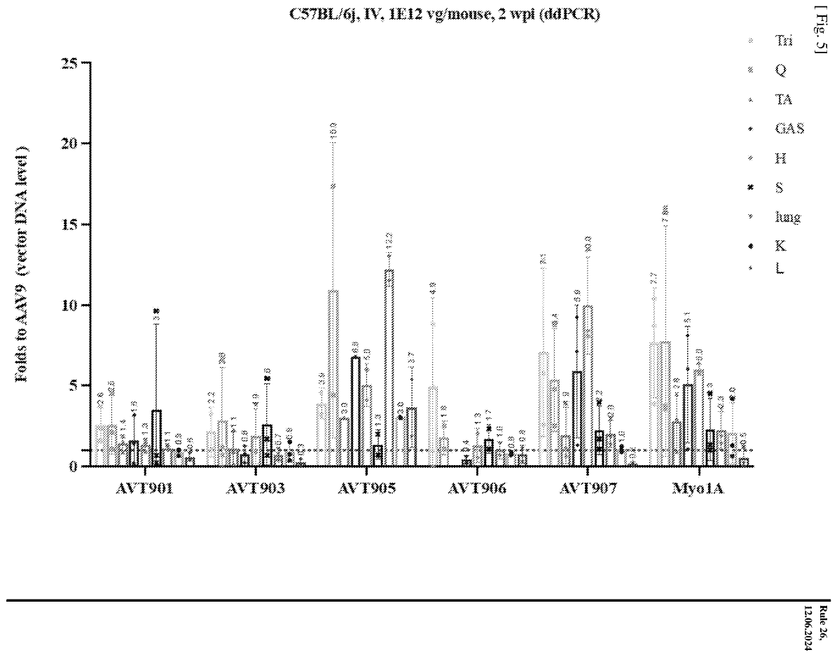

A schematic of the screening workflow is shown in FIG. 2. The top variant candidates selected for validation are shown in Table 3.

TABLE 3
Top variant candidates selected for validation.

| | Amino acid sequence of | | | | |
|---|---|---|---|---|---|
| | insert peptide (plus | | | | |
| | substitute sequence for | | | | |
| | wild-type positions | sequence | Yield | Library | Position- |
| AAV Alias | 586-588 for some). | Nucleotide | (vg/mL) | source | mer |
| AVT901 | HMNGRNN | catatgaatgggcgtaataat | 3.41E+11 | Lib#1 | 453-7 |
| | (SEQ ID NO: 37) | (SEQ ID NO: 38) | | | |
| AVT902 | PIIYTIIGI | cctcattatacgcatggtatt. | 1.42E+11 | Lib#1 | 453-7 |
| | (SEQ ID NO: 39) | (SEQ ID NO: 40) | | | |
| AVT903 | RGDTNNTNM | aggggtgatactaataatactaatatg | 4.94E+11 | Lib#3 | 588-9 |
| | (SEQ ID NO: 41) | (SEQ ID NO: 42) | | | |
| AVT905 | SRGDRPM | tctaggggtgatcggcctatg | 1.99E+11 | Lib#2 | 588-7 |
| | (SEQ ID NO: 43) | (SEQ ID NO: 44) | | | |
| AVT906 | LHKGIDI | ctgcataaggggattgatatt | 5,26E+11 | Lib#2 | 588-7 |
| | (SEQ ID NO: 45) | (SEQ ID NO: 46) | | | |
| AVT907 | HMNGRNN | catatgaatgggcgtaataat | 4.58E+11 | Lib#1 | 453-7 |
| | (SEQ ID NO: 47) | (SEQ ID NO: 48) | | | |
| | GPGRGDQTTL | ggccccggcagaggagaccagaccaccctg | | | 585-7 |
| | (SEQ ID NO: 49) | (SEQ ID NO: 50) | | | |
| AVT908 | SRGDRPM000 | tccagaggcgaccggcctatg | 1.48E+11 | Lib#2 | 588-7 |
| | (SEQ ID NO: 51) | (SEQ ID NO: 52) | | | |
| AVT909 | GVNGRST | gytytgaatgggaggagtact | 1.63E+11 | Lib#2 | 588-7 |
| | (SEQ ID NO: 53) | (SEQ ID NO: 54) | | | |
| AVT910 | GVNGRQV | ggggttaatgggcggcaggtg | 1.12F+11 | Lib#2 | 588-7 |
| | (SEQ ID NO: 55) | (SEQ ID NO: 56) | | | |
| AVT911 | QTYTHNK | cagaogtatacycataataag | 2.62E+11 | Lib#1 | 453-7 |
| | (SEQ ID NO: 57) | (SEQ ID NO: 58) | | | |
| AVT912 | QTYTIINK | cagacgtatacgcataataag | 1.53E+10 | Lib#1 | 453-7 |
| | SEQ ID NO: 59) | (SEQ ID NO: 60) | | | |
| | SRGDRPM | tctaggggtgatcggcctatg | | Lib#2 | 588-7 |
| | (SEQ ID NO: 61) | (SEQ ID NO: 62) | | | |
| AVT913 | ENRRGDTNAL | caggagaacaggagaggagacacgaacgctctggca | 3.48E+11 | Lib#4 | 585-7 |
| | (SEQ ID NO: 63) | (SEQ ID NO: 64) | | | |
| AVT914 | NNSRGDHNSL | cagaataactcgagaggagaccataactcgctggca | 2.81F+11 | Lib#4 | 585-7 |
| | (SEQ ID NO: 65) | (SEQ ID NO: 66) | | | |
| AVT915 | NNSRGDYNSL | cagaataacagtagaggagactataacagtctgyca | 2.83E+11 | Lib#4 | 585-7 |
| | (SEQ ID NO: 67) | (SEQ ID NO: 68) | | | |
| AVT916 | NNLRGDHNSL | cagaataacctgagaggagaccataactctctggca | 2.32E+11 | Lib#4 | 585-7 |
| | (SEQ ID NO: 69) | (SEQ ID NO: 70) | | | |
| AVT917 | NNARGDHNSL | cagaataacgctagaggagaccataacagtctygca | 3.10E+11 | Lib#4 | 585-7 |
| | (SEQ ID NO: 71) | (SEQ ID NO: 72) | | | |
| AVT918 | GNIRGDHNTL | cagggtaacattagaggagaccataacactctggca | 3.23E+11 | Lib#4 | 585-7 |
| | (SEQ ID NO: 73) | (SEQ ID NO: 74) | | | |
| AVT919 | MNVRGDTNKL | cagatgaacgtgagaggagacactaacaagctggca | 8.17E+10 | Lib#4 | 585-7 |
| | (SEQ ID NO: 75) | (SEQ ID NO: 76) | | | |
| AVT920 | TNMRGDIINAL | cagacgaacatgayaggagaccataacgcgctggca | 2.77E+11 | Lib#4 | 585-7 |
| | (SEQ ID NO: 77) | (SEQ ID NO: 78) | | | |
| AV9ML001 | NNNRGDNNYL | aacaacaacagaggcgacaacaactacctc | | Lib#4 | 585-7 |
| | (SEQ ID NO: 79) | (SEQ ID NO: 100) | | | |
| AV9ML002 | NNNRGDIINTL | laacaacaacagaggcgaccacaacaccctc | | Lib#4 | 585-7 |
| | (SEQ ID NO: 80) | (SEQ ID NO: 101) | | | |
| AV9ML004 | NNERGDRNSL | aacaacgaaagaggcgacagaaacagcctc: | | Lib#4 | 585-7 |
| | (SEQ ID NO: 81) | (SEQ ID NO: 102) | | | |
| AV9ML005 | NNSRGDIINTL | aacaacagcagaggcgaccacaacaccctc | | Lib#4 | 585-7 |
| | (SEQ ID NO: 82) | (SEQ ID NO: 103) | | | |
| AV9ML007 | ENKRGDANSL | gaaaacaaaagaggcgaccacaacagcctc | | Lib#4 | 585-7 |
| | (SEQ ID NO: 83) | (SEQ ID NO: 104) | | | |
| AV9ML012 | INSRGDHNVL | atcaucagcagaggcgaccacaacgtccto | | Lib#4 | 585-7 |
| | (SEQ ID NO: 84) | (SEQ ID NO: 105) | | | |
| AV9ML014 | NNNRGDYNSL | aacaacaacagaggcgactacaacagcctc | | Lib#4 | 585-7 |
| | SEQ ID NO: 85) | (SEQ ID NO;106) | | | |
| AV9ML016 | RGDKLSI | agaggcgacaaactcagcatc | | Lib#2 | 588-7 |
| | (SEQ ID NO: 86) | (SEQ ID NO: 107) | | | |
| AV9ML018 | LNINNSFTK | ctcaacaccaacaacagcttcaccaaa | | Lib#3 | 588-9 |
| | (SEQ ID NO: 87) | (SEQ ID NO: 108) | | | |
| AV9 (i.e., | | | 4.37E+11 | | |
| wild-type | | | | | |
| AAV9) | | | | | |

### Example 2: Characterization of AAV9-Derived Capsids Mediated Transgene Expression in C57BL/6J Mice

Five variants (AVT901, AVT903, AVT905, AVT906, AVT907) plus two controls (AAV9 and MyoAAV 1A (1A)) were individually packaged with green fluorescent protein (GFP) transgene driven by Chicken β-actin (CB) promoter. All 7 AAVs were produced in HEK293 cells by triple plasmid transfection with PEIpro (Polyplus). Three days after transfection, the cells were harvested for lysis, Benzonase (Sigma, E1014-25KU) treatment and proceeded to iodixanol gradient ultracentrifugation purification method. After buffer exchange using PBS with 0.001% F68, the AAV was filtered through 0.22 m. The titer was measured by ddPCR using ITR primer and probes. The purity of AAV was assessed by SDS-PAGE.

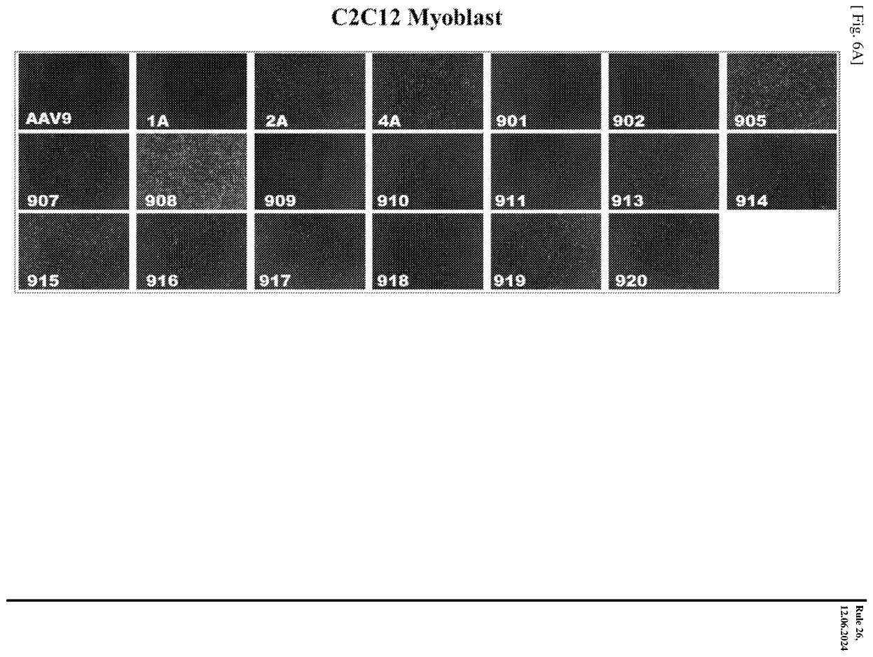

Eight-week-old C57BL/6J mice were intravenously injected with those 7 AAVs (1e12 vg/mouse). Two weeks post injection (wpi), the tissues were harvested for ex vivo imaging to detect native GFP expression (FIG. 3). AVT907, AVT905 and AVT903 showed dramatically stronger GFP intensity in skeletal muscles than AAV9 did. Specifically, AVT907>AVT905>AVT903>AAV9 in triceps and gastrocnemius, AVT907≈MyoAAV 1A>AVT905>AVT901>AVT903>AAV9≈AVT906 in quadriceps, AVT907>AVT905≈AVT903>AAV9 in tibialis anterior. AVT907 and AVT905 showed stronger GFP intensity in the heart than AAV9 did. AVT905 showed the strongest GFP signal in the liver, followed by AAV9. AVT903 showed weaker GFP in the liver than AAV9 did. No visible signal was captured in the liver for AVT907. For all of the tested variants and AAV9, no visible signal was captured in the lung, spleen and kidney.

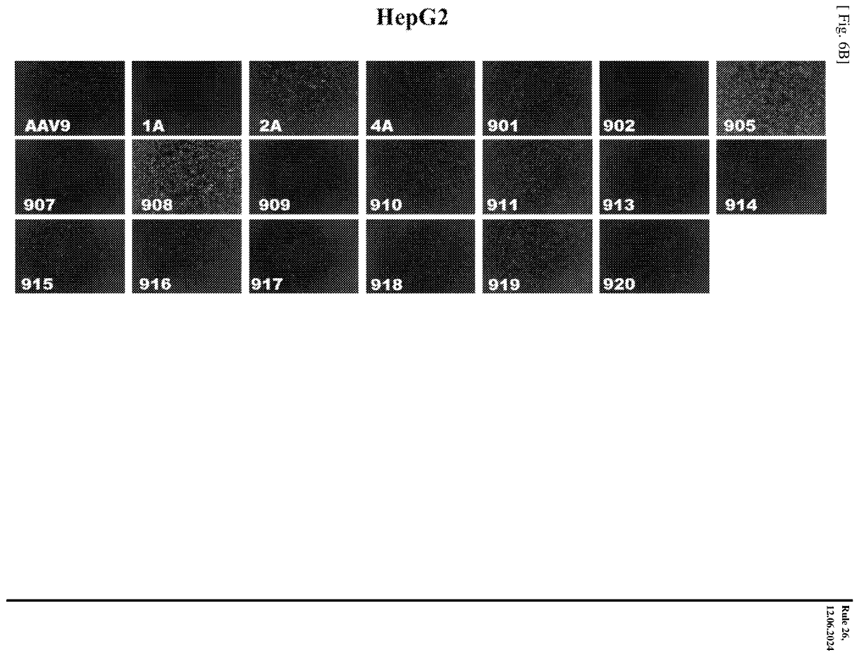

Then the total RNA was isolated from tissues using Trizol and treated with DNase I. After reverse transcription (RT), transgene mRNA was quantified by real-time qPCR. GFP and mouse GAPDH primers/probes were used for RT-qPCR (FIG. 4). The normalized level of AAV9-mediated GFP expression was set as 1.0 for comparison (FIG. 4). Compared to AAV9-mediated transgene level, AVT907-mediate transgene level was 17~37-fold in the skeletal muscles and 10-fold in the heart, AVT905-mediate transgene level was 3~12-fold in the skeletal muscles, 2-fold in the heart, 3-fold in the liver, 3-fold in the spleen and 5-fold in the lung, and AVT901-mediated transgene level was 4~6-fold in the skeletal muscles.

Genomic DNA from triceps, quadriceps, gastrocnemius, tibialis anterior, heart, spleen, kidney and liver were also isolated two weeks post injection (wpi) using DNeasy Blood &Tissue kit (Qiagen). The AAV vector genome copies in those tissues were measured by Droplet Digital PCR (ddPCR) with GFP and mouse TFRC primers/probes (FIG. 5). The AAV9 vector genome level was set as 1.0 (FIG. 5). Compared to AAV9 vector genome copies, AVT907's vector genome copies were 2~7-fold in the skeletal muscles and 10-fold in the heart, AVT905's vector genome copies were 3~11-fold in the skeletal muscles, 5-fold in the heart, 4-fold in the liver, 3-fold in the kidney and 12-fold in the lung, and AVT901's vector genome copies were 3-fold in the triceps and quadriceps.

### Example 3: Novel AAV9 Variants Mediated Transgene Expression In Vitro

16 variants (AVT901, AVT902, AVT905, AVT907, AVT908, AVT909, AVT910, AVT911, AVT913, AVT914, AVT915, AVT916, AVT917, AVT918, AVT919, AVT920) and four controls (AAV9 MyoAAV 1A, MyoAAV 2A, MyoAAV 4A) were individually packaged with a transgene expressing GFP and firefly luciferase (Fluc) linked with 2A and driven by chicken β-actin (CB) promoter. The production and titer analysis were perform as described previously. AVT905 and AVT908 share the same amino acid sequence with different codons. C2C12 myoblast and HepG2 cells were seeded into 96-well plates and incubated (1e6 vg/cell) with indicated AAV vectors containing CB-Fluc-2A-GFP transgene.

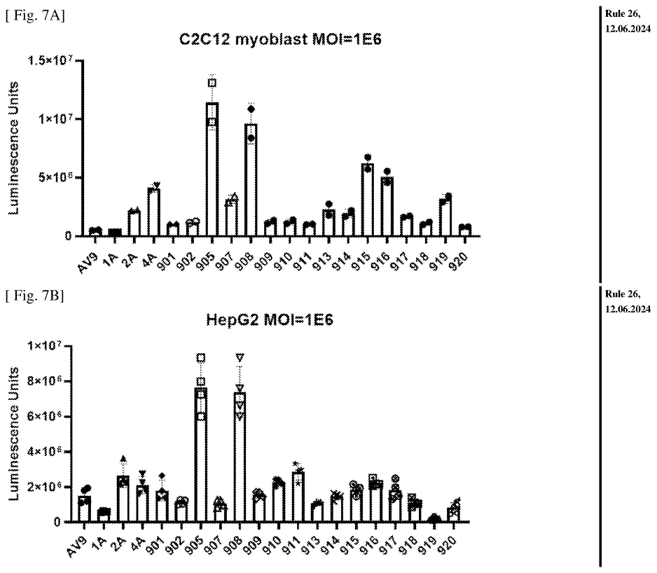

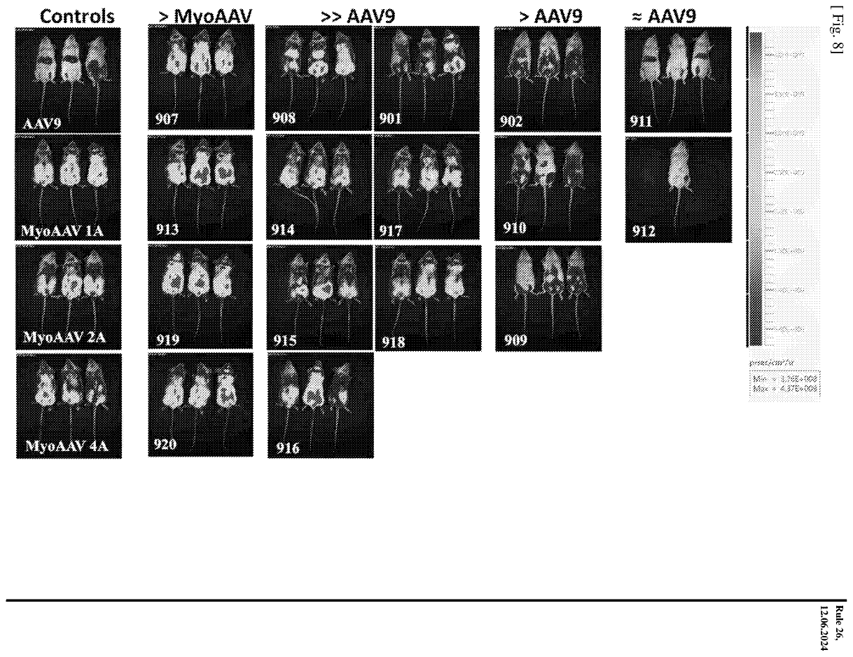

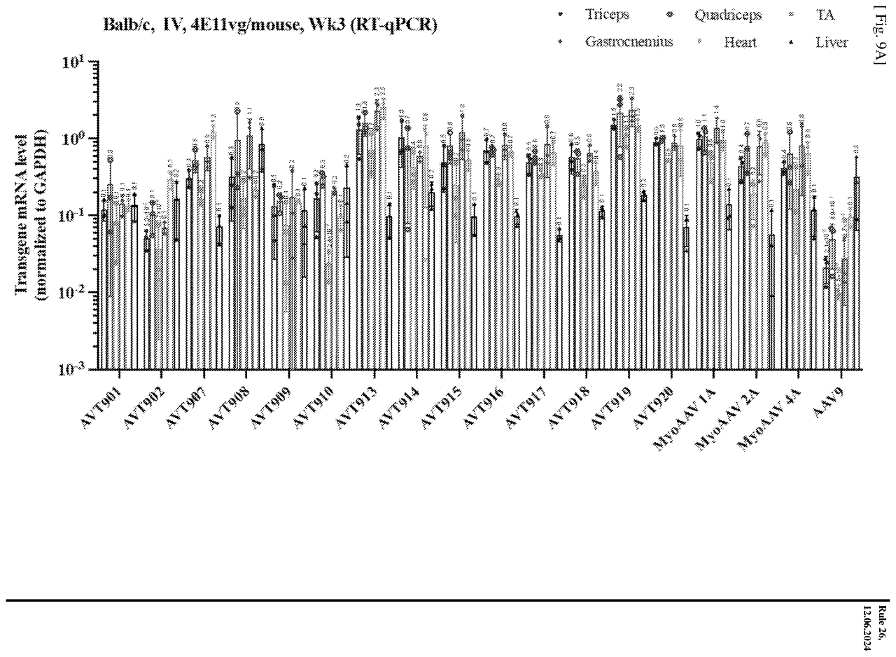

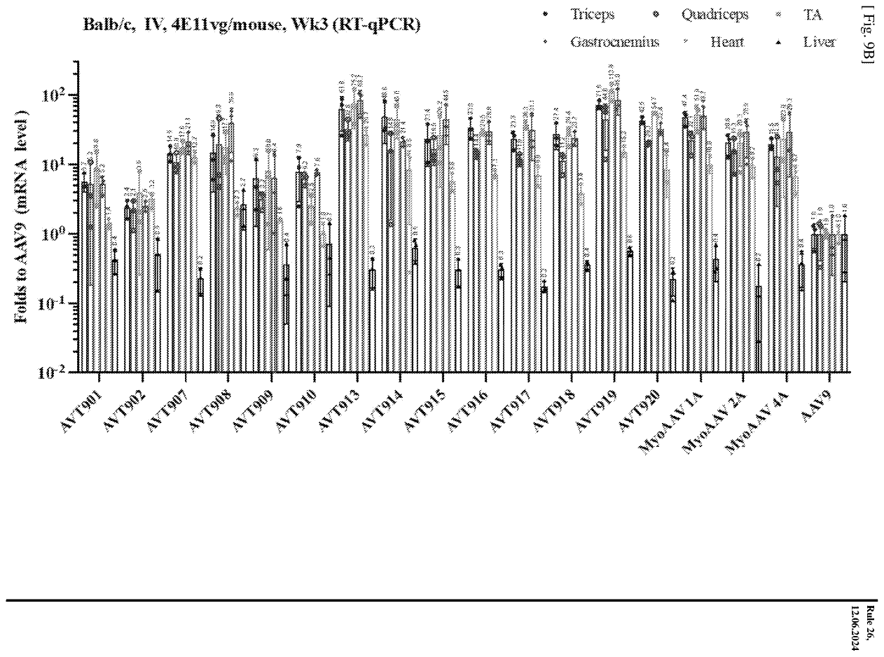

Twenty-four hours after transduction, the native GFP imaging was captured with the same conditions (FIGS. 6A and 6B). AVT905 and AVT908 showed nearly 100% GFP positive in both C2C12 and HepG2 cells. No GFP signals were observed for AAV9, 1A, AVT901, AVT902, AVT909, AVT910 and AVT911 in C2C12 cells. No GFP signals were observed for AAV9, 1A, 4A, AVT902, AVT907, AVT909, AVT910, AVT913, AVT918 and AVT920 in HepG2 cells. Forty-eight hours after transduction, the cells were lysed and Fluc expression was quantified by luciferase assay system (Promega) (FIGS. 7A and 7B). AVT905 and AVT908 showed significantly stronger luminescence signals than other variants did. Compared to AAV9-mediated luciferase expression in C2C12 myoblasts, the luciferase expressions in C2C12 myoblast cells mediated by MyoAAV 1A, MyoAAV 2A, MyoAAV 4A AVT901, AVT902, AVT905, AVT907, AVT908, AVT909, AVT910, AVT911, AVT913, AVT914, AVT915, AVT916, AVT917, AVT918, AVT919, and AVT920 were 0.7-fold, 4.2-fold, 7.8-fold, 1.9-fold, 2.3-fold, 21.9-fold, 6.1-fold, 18.5-fold, 2.3-fold, 2.4-fold, 1.9-fold, 4.4-fold, 3.7-fold, 12-fold, 9.7-fold, 3.2-fold, 2.1-fold, 6.1-fold, and 1.5-fold, respectively. Compared to AAV9-mediated luciferase expression in HepG2 cells, the luciferase expression in HepG2 cells mediated by MyoAAV 1A, MyoAAV 2A, MyoAAV 4A AVT901, AVT902, AVT905, AVT907, AVT908, AVT909, AVT910, AVT911, AVT913, AVT914, AVT915, AVT916, AVT917, AVT918, AVT919, and AVT920 were 0.4-fold, 1.7-fold, 1.4-fold, 1.2-fold, 0.8-fold, 5.0-fold, 0.7-fold, 4.8-fold, 1.0-fold, 1.5-fold, 1.9-fold, 0.7-fold, 1.0-fold, 1.2-fold, 1.5-fold, 1.2-fold, 0.7-fold, 0.1-fold, and 0.6-fold, respectively.

### Example 4: Novel AAV9 Variants Mediated Transgene Expression in Balb/c Mice

16 variants (AVT901, AVT902, AVT907, AVT908, AVT909, AVT910, AVT911, AVT912, AVT913, AVT914, AVT915, AVT916, AVT917, AVT918, AVT919, AVT920) and four controls (AAV9 MyoAAV 1A, MyoAAV 2A, MyoAAV 4A) were individually packaged with a transgene expressing GFP and firefly luciferase (Fluc) linked with 2A and driven by chicken β-actin (CB) promoter.

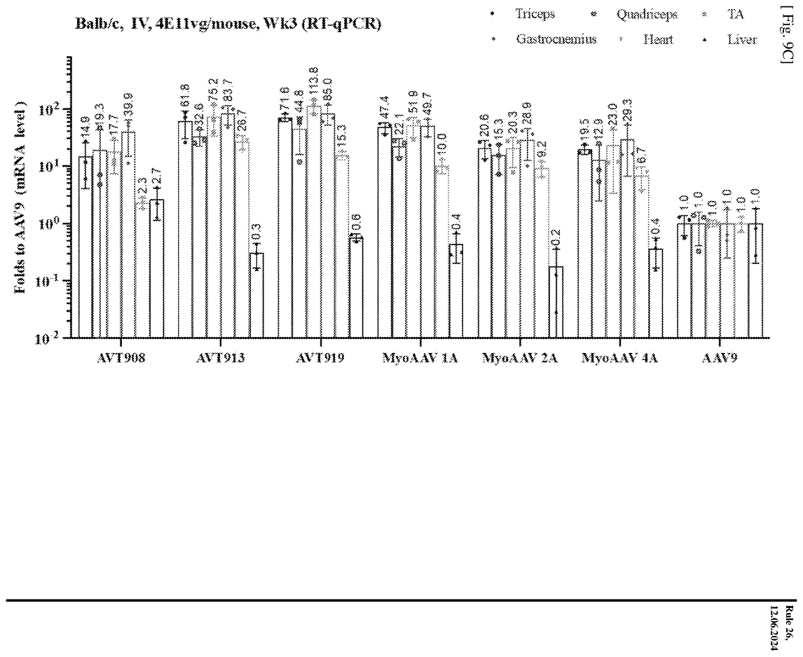

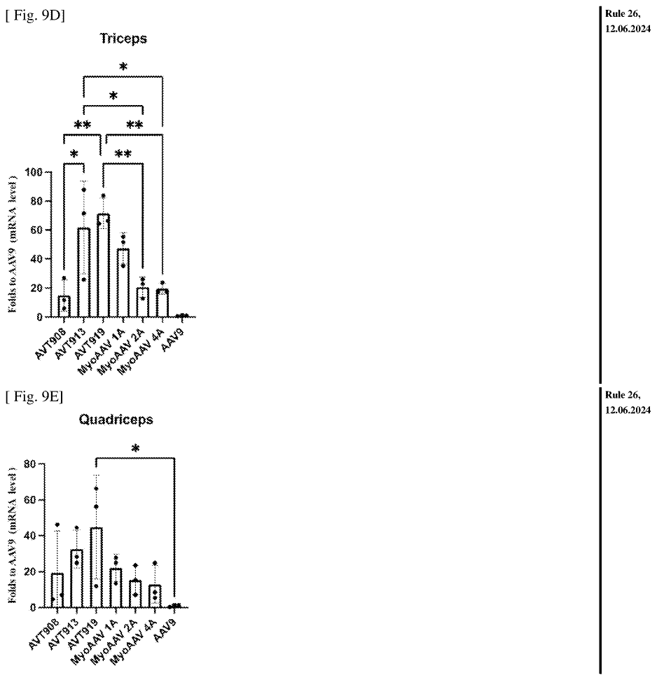

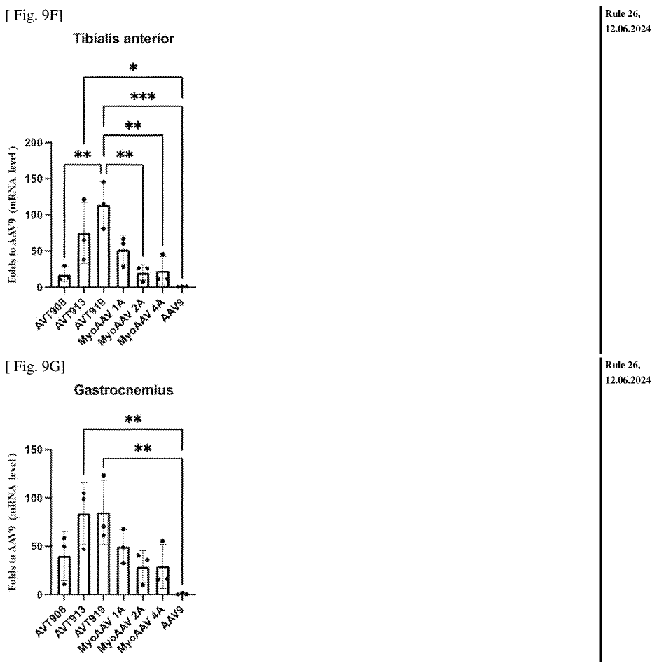

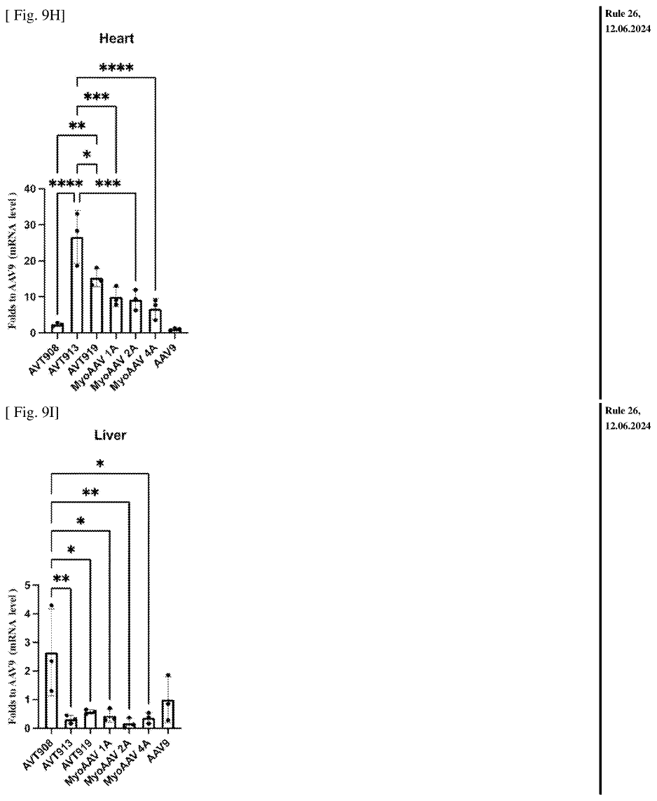

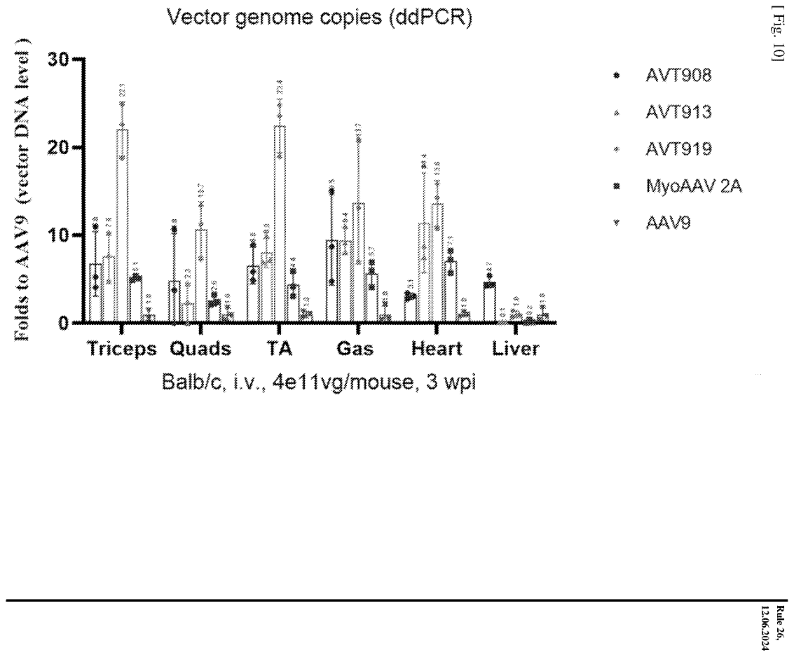

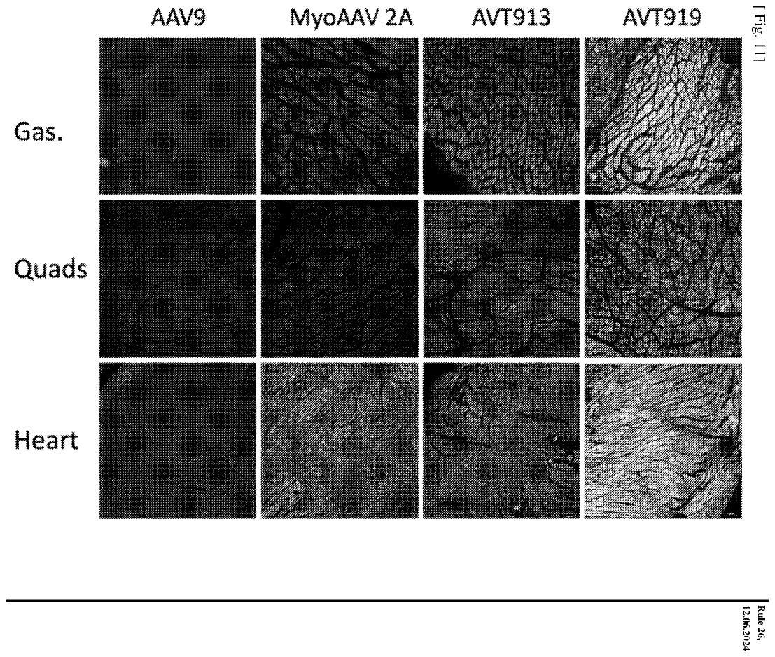

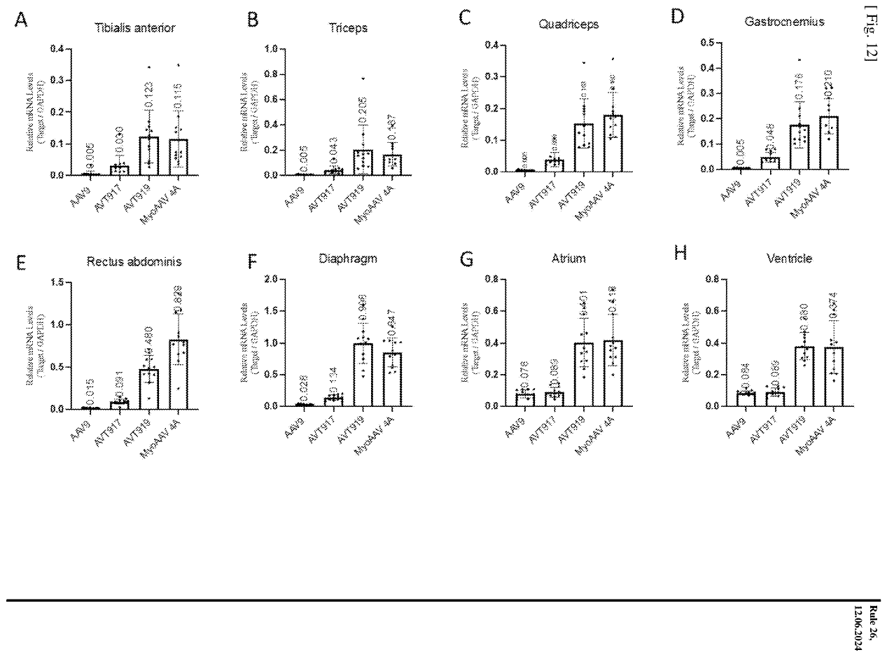

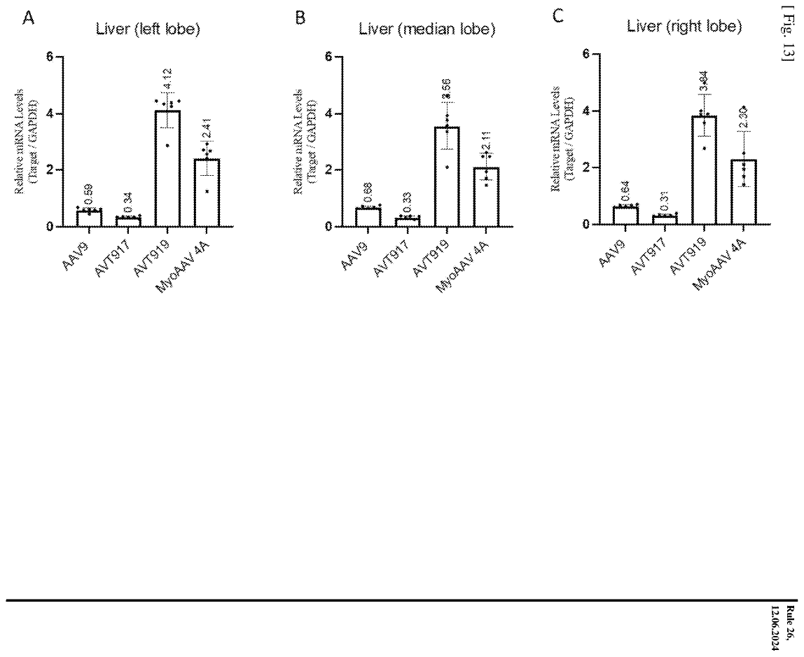

Eight-week-old Balb/c mice were intravenously (tail vein) injected with the 20 AAVs (4e11 vg/mouse). Three weeks post injection (wpi), the mice were intraperitoneal injected with luciferin. Ten minutes later, mice were proceeded to In vivo Imaging System (IVIS) to capture firefly luciferase expression (FIG. 8). Compared to AAV9-mediated luminescence signals in the skeletal muscles, AVT911 and AVT912 showed similar level, AVT902, AVT909 and AVT910 showed stronger luminescence signals, AVT901, AVT908, AVT914, AVT915, AVT916, AVT917 and AVT918 showed significantly stronger luminescence signals in the skeletal muscles. Notably, AVT907, AVT913, AVT919 and AVT920 showed stronger luminescence signals in the skeletal muscles compared to the published MyoAAV2A, a benchmark myotropic AAV capsid. In addition, AAV908 showed significantly stronger luminescence signals in the liver than AAV9 did. The tissues (triceps, quadriceps, gastrocnemius, tibialis anterior, heart and liver) were also harvested three weeks post injection (wpi) for the total RNA and genomic DNA isolation. The AAV-mediated transgene mRNA level was quantified and analyzed by RT-qPCR (FIG. 9A-9I). The mouse GAPDH (^{vIC®} label) was used as reference. Compared to AAV9 wild type, AVT919-mediated transgene expression levels were 72-fold, 45-fold, 113-fold, 85-fold, and 15-fold in triceps, quadriceps, tibialis anterior, gastrocnemius and heart, respectively (FIGS. 9B-9H), AVT913-mediated transgene expression levels were 62-fold, 33-fold, 75-fold, 84-fold, and 27-fold in triceps, quadriceps, tibialis anterior, gastrocnemius and heart, respectively (FIGS. 9B-9H), AVT908-mediated transgene expression levels were 15-fold, 19-fold, 18-fold, 40-fold, and 2-fold in triceps, quadriceps, tibialis anterior, gastrocnemius and heart, respectively (FIGS. 9B-9H). AVT908, AVT913 and AVT919 showed 270%, 30% and 60% of AAV9-mediated transgene expression level in the liver (FIGS. 9B, 9C and 9I). The AAV vector genome copies were analyzed by ddPCR (FIG. 10). The mouse TFRC (^{VIC®} label) was used as reference. The primers/probe (FAM label) against Flue was used for AAV genome. Compared to AAV9, the novel capsids AVT908, AVT913, and AVT919 all showed improvements in triceps, quadriceps, tibialis anterior, gastrocnemius, and heart muscle at genome DNA level, while AVT908 showed improvement in liver at genome DNA level (FIG. 10). Specifically, the genome DNA levels of AVT908, AVT913, and AVT919 in tibialis anterior were 6.8-fold, 7.6-fold, and 22.1-fold of the genome DNA level of AAV9, respectively (FIG. 10). The genome DNA levels of AVT913 and AVT919 in the heart were more than 10-fold of the genome DNA level of AAV9 (FIG. 10).

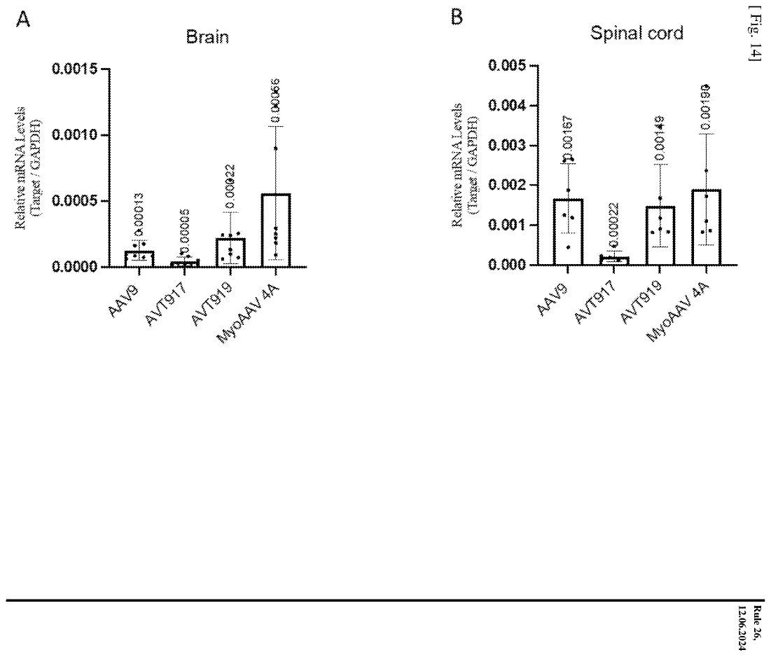

In a repeated mouse study, two variants (AVT913 and AVT919) and two control capsids (AAV9 and MyoAAV 2A) containing the same expression cassette (chicken β-actin promoter driven GFP and firefly luciferase linked with 2A sequence) were intravenously (tail vein) injected to eight-week-old male Balb/c mice. Three weeks after injection, the muscle tissues were harvested and processed for native GFP imaging. No visible or very weak imaging signal were observed in AAV9-treated mouse tissues (FIG. 11). Variant AVT917 showed the strongest GFP intensities in the muscle tissues. Specifically, AVT919>AVT913>MyoAAV 2A>AAV9 in the gastrocnemius and quadriceps, AVT919>AVT913≈MyoAAV 2A>AAV9 in the heart (FIG. 11).

### Example 5: Validation of Pooled Novel Capsids in NHP

The encoding sequences of 19 novel capsids and 5 published controls (Myo1A, Myo2A, Myo3A, Myo4A, and AAV9) were individually cloned to library backbone containing p41 promoter, AAV2 ITRs, a synthetic promoter and bGH polyA. The 24 capsids were individually produced in HEK293 cells by co-transfecting Rep2-AAP and Ad5 helper plasmids. After purification with iodixanol gradient ultracentrifugation method and filtration through 0.22 m, the titer was measured by ddPCR using ITR primers/probe. The purity was analyzed by SDS-PAGE.

An adult female cynomolgus macaque (4-year-old, ~3 kg) was intravenously injected with those pooled 24 capsids. Three weeks post injection (wpi), the tissues were collected for total RNA and genomic DNA isolation. The total RNA was treated with Dnase I to remove AAV genomes. The cap9 specific mRNA was reversely transcribed and amplified by PCR (RT-PCR). The genomic DNA was used for cap9 specific PCR. The pooled (for NHP injection) AAV vector DNA was amplified by PCR with minimal cycles. All the purified PCR products were subjected to NGS (NovaSeq 6000 S4 Reagent Kit v1.5, 300 cycles). The enrichment score (ES) was analyzed by customized pipeline.

The total 24 viral capsid mRNA transgene levels were compared to wild type AAV9 and shown as fold change (FC) (Table 4).

### Example 6: Multiplex Validation of Individual Novel Capsids in NHP

An adult male cynomolgus macaque (4.25 kg) was intravenously injected with four pooled capsids, i.e. two variants (AVT917 and AVT919), two control capsids (AAV9 and MyoAAV 4A) at the dose of 1E13 vg/kg/capsid. Each capsid contained cyno FXN coding sequences with different tags (Flag, AU1, V5, HA) under the control of chicken beta-actin (CB) promoter and CMV enhancer. The animal was orally administrated with methylprednisolone at the dose of 1 mg/kg/day three days before the injection of AAV for total 8 days. Three weeks post injection (wpi) of AAV, the animal was anesthetized and sacrificed. The tissues were collected for total RNA and genomic DNA isolation. For sampling, multiple locations of the same tissues were collected. The mRNA was reversely transcribed using HiScript III 1V cDNA synthesis kit (+gDNA wiper) (Vazyme, R412). The transgene specific transcripts were quantified by qPCR using primers/probe against the tags. The endogenous macaque GAPDH housekeeping gene was used for normalization. The Bio-Rad CFX96 real-time PCR detection system was used to acquire and analyze the data. Relative transgene mRNA levels were calculated using the 2^{-ΔCt} method. To determine the AAV vector genome copies in the tissues, 10~300 ng purified genomic DNA was used in the 20 μL ddPCR reaction mix according to the instruction of ddPCR Supermix for Probes (no dUTP, Cat. No. 1863025, Bio-Rad). The primers/probe (FAM labelled) against the tags were used for vector genome detection and the RNase P primers/probe (VIC labelled, Cat. No. 4403328, ThermoFisher Scientific) were used as macaque genome copy number reference. The QX200 droplet generator and Reader (Bio-Rad) were used for data acquisition and analysis. The AAV9-mediated transgene expression level was set as 1.0. The fold change over AAV9 was calculated to show the improvement of capsid AVT917 and AVT919.

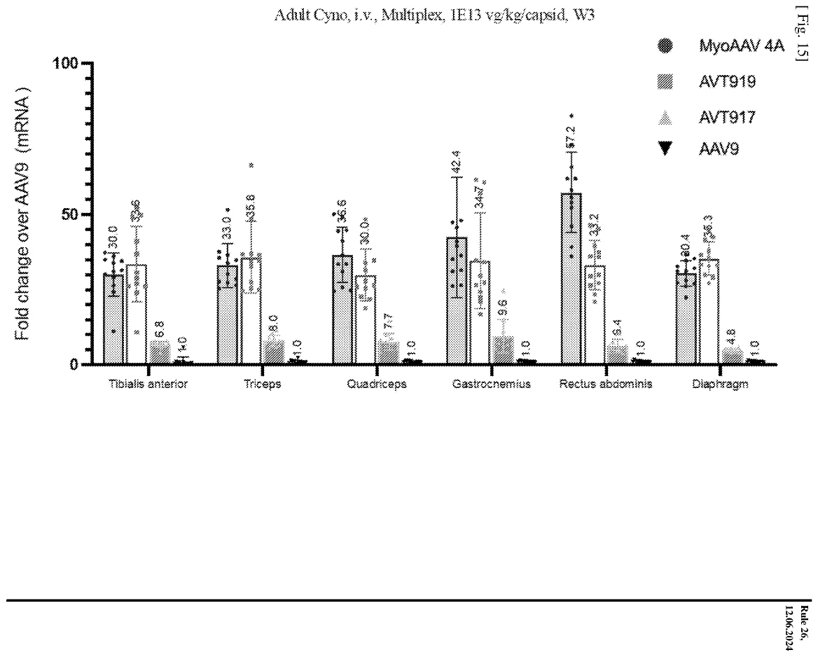

By comparing the transgene expression at mRNA level, AVT919 and control capsid MyoAAV 4A showed significant higher transduction in the tibialis anterior, triceps, quadriceps, gastrocnemius, rectus abdominis, diaphragm, atrium and ventricle of heart than AAV9 did (FIG. 12). AVT917 also showed higher expression in the muscle tissues than AAV9 did (FIG. 12). Briefly, AVT919≈MyoAAV 4A>AVT917>AAV9 in the muscle tissues of NHP.

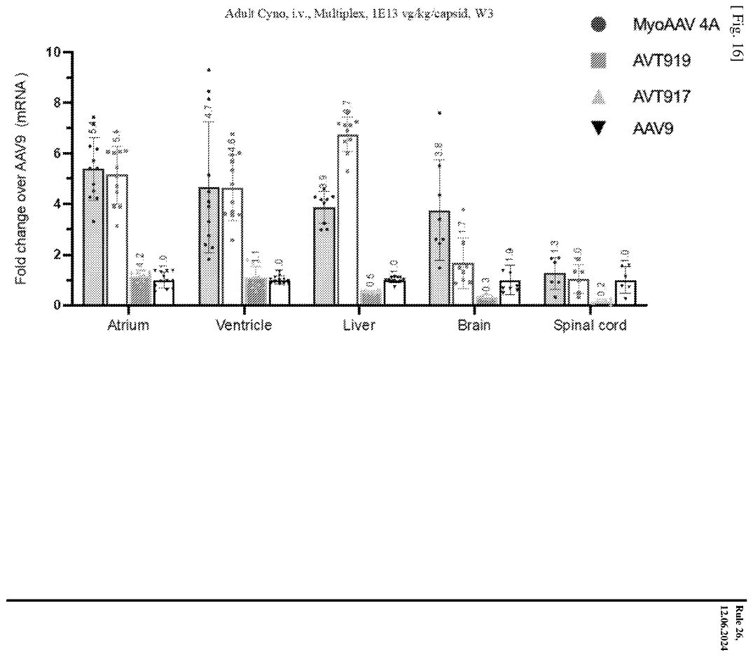

In the liver of NHP, AVT919 showed highest expression level in all the lobes of liver (FIG. 13). Briefly, AVT919>MyoAAV 4A>AAV9>AVT917 in the monkey liver after intravenously administration (FIG. 13).

No dramatic difference was observed in the brain and spinal cord tissues for AVT919, MyoAAV4A and AAV9 (FIG. 14). AVT917 showed relatively lower transgene expression in the brain and spinal cord after intravenously administration (FIG. 14).

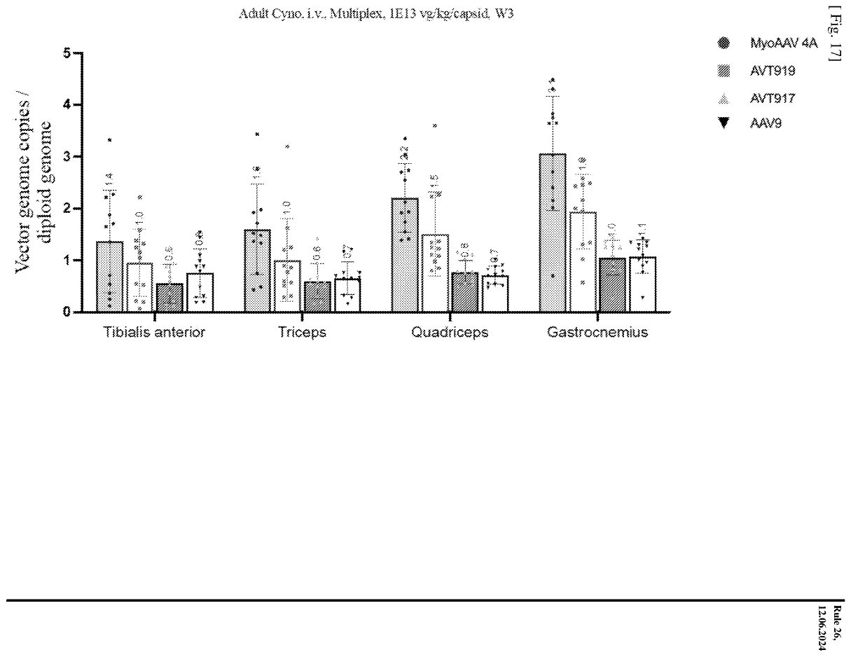

Compared to AAV9 (down triangle in FIG. 15), variant AVT919 (square in FIG. 15) showed 33.6-fold, 35.8-fold, 30-fold, 34.7-fold, 33.2-fold, 35.3-fold improvements at mRNA level in the tibialis anterior, triceps, quadriceps, gastrocnemius, rectus abdominis, diaphragm, respectively (FIG. 15).

Compared to AAV9 (down triangle in FIG. 15), variant AVT917 (up triangle in FIG. 15) showed 6.8-fold, 8.0-fold, 7.7-fold, 9.6-fold, 6.4-fold, 4.8-fold improvements at mRNA level in tibialis anterior, triceps, quadriceps, gastrocnemius, rectus abdominis, diaphragm, respectively (FIG. 15).

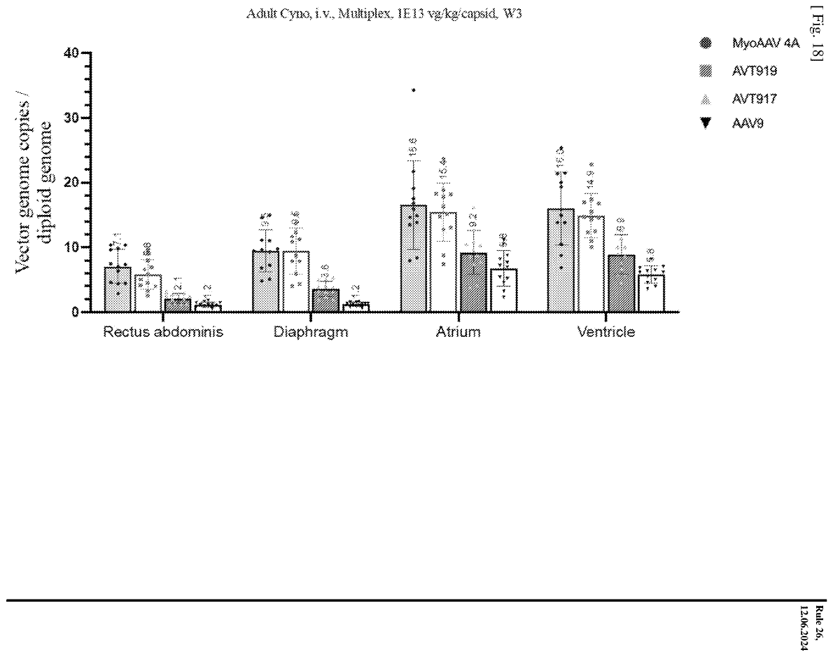

Compared to AAV9 (down triangle in FIG. 16), variant AVT919 (square in FIG. 16) showed 5.1-fold, 4.6-fold, 6.7-fold, 1.7-fold, 1.0-fold, improvements at mRNA level in the atrium, ventricle, liver, brain, spinal cord, respectively (FIG. 16).

Variant AVT917 (up triangle in FIG. 16) showed 1.2-fold, 1.1-fold, 0.5-fold, 0.3-fold, 0.2-fold of AAV9 at mRNA level in the atrium, ventricle, liver, brain, spinal cord, respectively (FIG. 16).

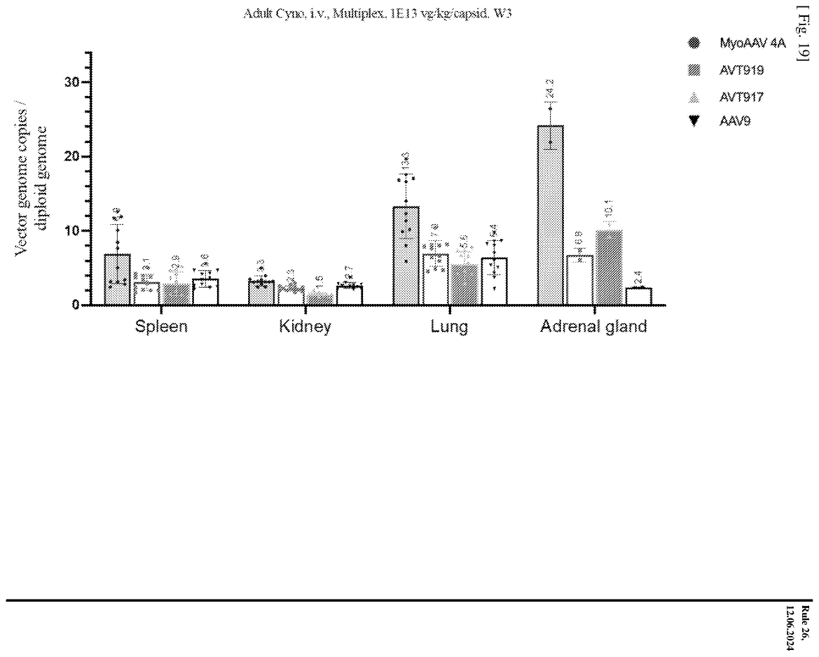

By comparing the vector genome copies, AVT919 (square in FIG. 17) and control capsid MyoAAV 4A (filled circle in FIG. 17) showed significant higher transduction in the tibialis anterior, triceps, quadriceps, gastrocnemius than AAV9 did (FIG. 17). Three weeks after intravenous administration at the dose of 1E13 vg/kg in the NHP, AVT919 showed the vector genome copies per diploid genome of 1.0, 1.0, 1.5, 1.9 in the tibialis anterior, triceps, quadriceps, gastrocnemius, respectively (FIG. 17 square symbol).

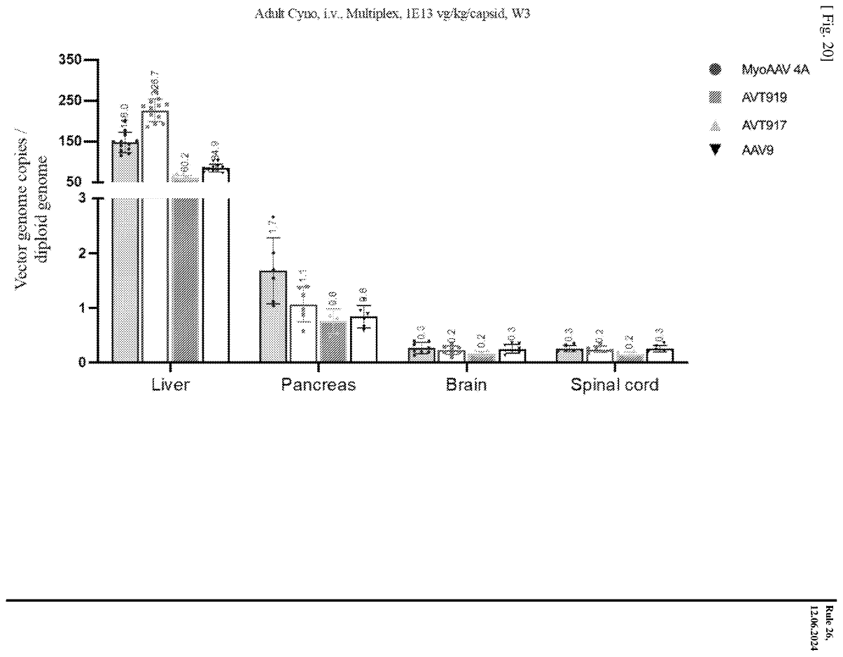

By comparing the vector genome copies, AVT919 and control capsid MyoAAV 4A showed significant higher transduction in the rectus abdominis, diaphragm, atrium and ventricle of heart than AAV9 did (FIG. 18). Three weeks after intravenous administration at the dose of 1E13 vg/kg in the NHP, AVT919 showed the vector genome copies per diploid genome of 5.8, 9.4, 15.8, 14.9 in the rectus abdominis, diaphragm, atrium and ventricle of heart (FIG. 18 square symbol).

AVT917 (up triangle in FIG. 18) showed higher transduction in the rectus abdominis, diaphragm, atrium and ventricle of heart than AAV9 did in the NHP (FIG. 18).

Compared to AAV9, variants AVT917 and AVT919 showed similar transduction at DNA level in the spleen, kidney and lung (FIG. 19).

AVT917, AVT919 and control capsid MyoAAV 4A showed higher transduction at DNA level in the adrenal gland than AAV9 did (FIG. 19). Specifically, three weeks after intravenous administration at the dose of 1E13 vg/kg in the NHP, MyoAAV4A, AVT919, AVT917 and AAV9 showed the vector genome copies per diploid genome of 24.2, 6.8, 10.1, 2.4, respectively, in the adrenal gland (FIG. 19).

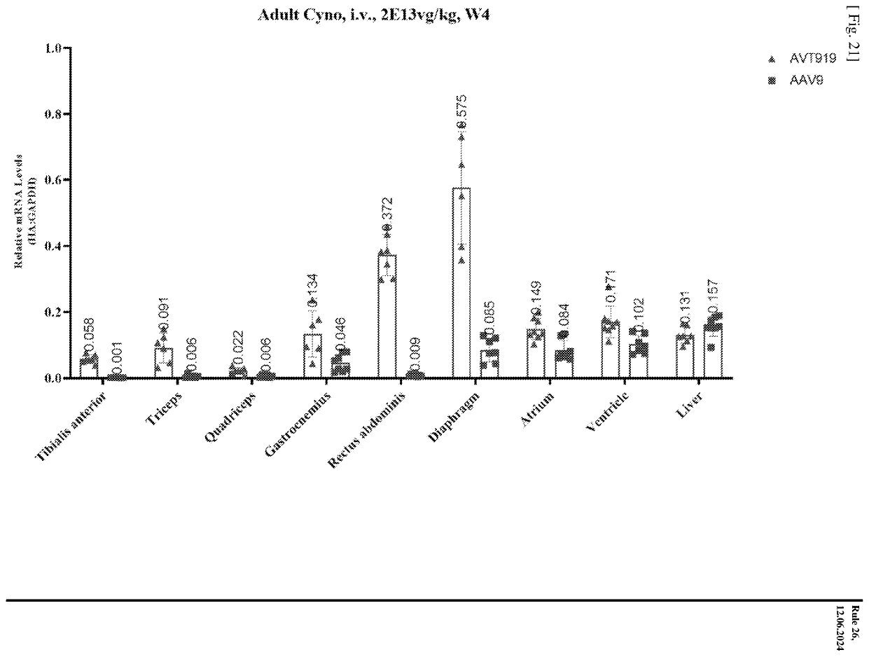

Compared to AAV9, variants AVT917 and AVT919 showed similar transduction at DNA level in the pancreas, brain and spinal cord (FIG. 20).

AVT919 showed dramatically higher vector genome copies in the liver of NHP than control capsids MyoAAV 4A and AAV9 did (FIG. 20). Specifically, three weeks after intravenous administration at the dose of 1E13 vg/kg in the NHP, MyoAAV4A, AVT919, AVT917 and AAV9 showed the vector genome copies per diploid genome of 148.0, 226.7, 60.2, 84.9, respectively, in the liver (FIG. 20).

### Example 7: Singleplex Validation of Individual Novel Capsids in NHPs

One 4-year-old female Cynomolgus monkey (3.35 kg) was intravenously injected with capsid AAV9 at the dose 2E13 vg/kg. One 5-year-old female Cynomolgus monkey (3.0 kg) was intravenously injected with variant AVT919 at the same dose. Both animals had negative pre-existing neutralizing anti-AAV9 capsid antibodies. Both AAV9 and AVT919 capsids contained the same AAV genome sequence which is chicken beta-actin promoter driven Cyno FXN transgene tagged with hemagglutinin (HA). Four weeks after the intravenous injection, the animal was anesthetized and sacrificed. The tissues were collected for total RNA extraction. For sampling, multiple locations of the same tissues were collected. The mRNA was reversely transcribed using HiScript III 1V cDNA synthesis kit (+gDNA wiper) (Vazyme, R412). The transgene specific transcripts were quantified by RT-qPCR using primers/probe against the HA tag. The endogenous macaque GAPDH housekeeping gene was used for normalization. The relative transgene mRNA levels were calculated using the 2^{-ΔCt} method.

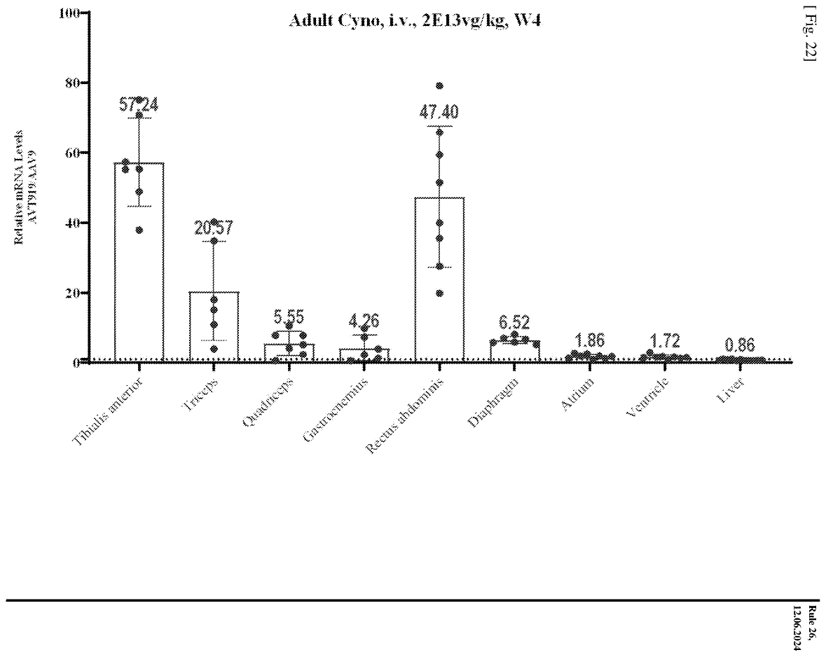

Compared to AAV9 (square in FIG. 21), variant AVT919 showed significantly improved transgene expression at mRNA level in the skeletal muscles, including tibialis anterior, triceps, quadriceps, gastrocnemius, rectus abdominis, diaphragm and heart (FIG. 21).

Specifically, AVT919 showed 57-fold, 21-fold, 6-fold, 4-fold, 47-fold, 7-fold, 2-fold improvement over AAV9 at mRNA level in the tibialis anterior, triceps, quadriceps, gastrocnemius, rectus abdominis, diaphragm and heart, respectively (FIG. 22).

To detect the AAV-mediated transgene protein products, the collected tissues were processed for immunostaining with rabbit anti-HA monoclonal primary antibody (Cat. No. 37245, Cell Signaling Technology). The 1: 5000 diluted peroxidase AffiniPure goat anti-rabbit IgG (H+L) secondary antibody (Jackson, Cat. No. 111-035-003) and DAB substrate were used for staining (brown color).

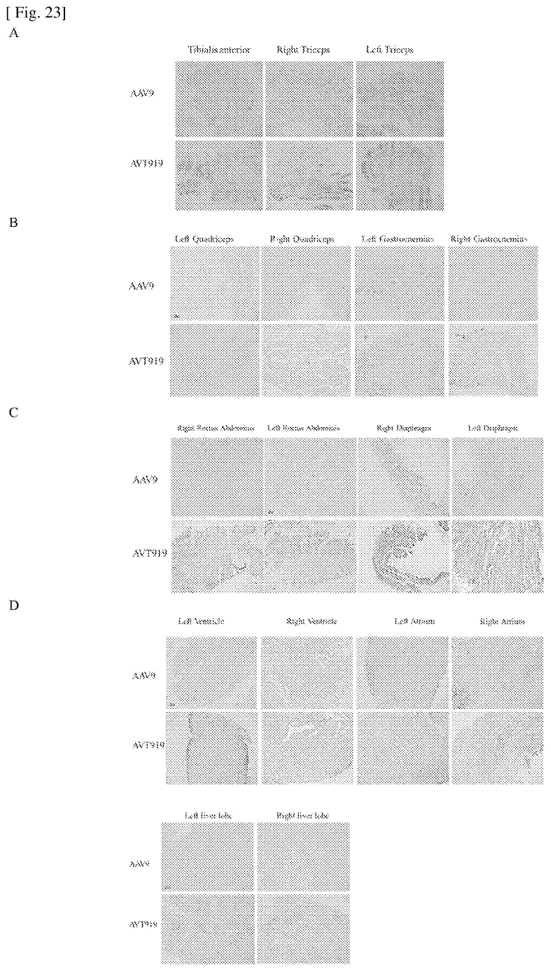

Four weeks after intravenous administration of AAV at the dose of 2E13 vg/kg in the NHPs, AAV9 showed weak or low transgene expression at protein level in the tibialis anterior, triceps, quadriceps, gastrocnemius, rectus abdominis (FIG. 23A-C). In contrast, variant AVT919 showed strong or high transgene expression cross muscle tissues, including tibialis anterior, triceps, quadriceps, gastrocnemius, rectus abdominis, diaphragm and heart (FIG. 23 A-D).

Compared to AAV9, variant AVT919 also showed higher transgene expression at protein level than in the liver of NHP (FIG. 23E).

TABLE 4
Fold change over AAV9 (mRNA level) after validation in NHP.

| | 586-588 of | | | | | | | | pecto- | | | | middle | middle | | |
|---|---|---|---|---|---|---|---|---|---|---|---|---|---|---|---|---|
| Variant | AAV9 VP1 | tibialis | | | | gastroc- | rectus | | ralis | | ven- | | front | rear | front | rear |
| name | reconstituted | anterior | biceps | tricep | quadriceps | nemius | abdominis | diaphragm | major | atrium | tricle | liver | brain | brain | brain | brain |
| AV9ML001 | NWARGDNNYL | 10.37 | 10.81 | 17.14 | 2.91 | 5.19 | 10.26 | 10.65 | 11.76 | 5.79 | 7.68 | 0.78 | 0.49 | 0.72 | 0.49 | 0.80 |
| | (SEQ ID NO: 79) | | | | | | | | | | | | | | | |
| AV9ML002 | NNNNGDIINTL | 10.97 | 10.13 | 18.18 | 5.73 | 5.88 | 13.63 | 12.10 | 13.91 | 6.66 | 11.06 | 1 49 | 1.32 | 1.05 | 4.37 | 0.92 |
| | (SEQ ID NO: 80) | | | | | | | | | | | | | | | |
| AVT914 | NNSRGDANSL | 37.22 | 35.08 | 69:95 | 63.15 | 20.45 | 58.24 | 37.70 | 53.93 | 14.59 | 28.29 | 2.67 | 1.97 | 2.00 | 2.67 | 0.73 |
| | (SEQ ID NO: 65) | | | | | | | | | | | | | | | |
| AV9ML004 | NNERGORYSL | 24.91 | 24.58 | 38:06 | 19.23 | 14.80 | 37.51 | 22.78 | 34.85 | 8.69 | 18.19 | 1.69 | 2.35 | 1.29 | 2.92 | 0.72 |
| | (SEQ ID NO: 81) | | | | | | | | | | | | | | | |
| AV9ML005 | NNSRGDHSTL | 22.45 | 21.25 | 38.34 | 16.33 | 12.36 | 30.49 | 21.79 | 30.33 | 10.73 | 19.94 | 1.52 | 1.55 | 1.23 | 10.49 | 1.43 |
| | (SEQ ID NO: 82) | | | | | | | | | | | | | | | |
| AVT916 | INNLRGDHNSL | 33.70 | 27.43 | 65:44 | 30.70 | 16.24 | 46.02 | 37.68 | 42.81 | 14.54 | 25.15 | 2.56 | 1.34 | 1.77 | 2.18 | 1.86 |
| | (SEQ ID NO: 69) | | | | | | | | | | | | | | | |
| AV9ML007 | ENKRGDHNSL | 18.62 | 17.24 | 35.50 | 10.09 | 10.93 | 27.55 | 19.34 | 24.89 | 8.58 | 17.75 | 1.02 | 0.98 | 0.60 | 1.77 | 0.72 |
| | (SEQ ID NO: 33) | | | | | | | | | | | | | | | |
| AVT917 | NNARGDHNSL | 37.76 | 33.38 | 69.64 | 27.24 | 21.19 | 49.94 | 29.68 | 50.65 | 12.55 | 28.84 | 2.46 | 1.68 | 2.16 | 2.05 | 1.77 |
| | (SEQ ID NO: 71) | | | | | | | | | | | | | | | |
| AVT913 | ENRRGDTNAL | 52.03 | 38.10 | 67.55 | 41.66 | 26.68 | 75.79 | 37.67 | 65.08 | 17.22 | 30.32 | 2.28 | 1.79 | 1.72 | 6.03 | 1.60 |
| | (SEQ ID NO: 63) | | | | | | | | | | | | | | | |
| AV1919 | MNVRGDINKL | 48.67 | 43.73 | 106.58 | 13.44 | 26.16 | 111.15 | 79.04 | 82.14 | 11.27 | 29.49 | 1.45 | 1.23 | 1.53 | 5.51 | 1.31 |
| | (SEQ ID NO: 75) | | | | | | | | | | | | | | | |
| AV1918 | GNIRGDHNTL | 26.44 | 21.33 | 31.80 | 12.78 | 13.42 | 30.66 | 27.42 | 33.12 | 10.74 | 16.48 | 2.42 | 2.00 | 1.65 | 9.42 | 2.52 |
| | (SEQ ID NO: 73) | | | | | | | | | | | | | | | |
| AV9ML012 | INSRGDIINVL | 30.64 | 37.86 | 54.16 | 12.77 | 16.80 | 51.36 | 39.13 | 49.57 | 9.05 | 19.04 | 0.47 | 0.41 | 0.25 | 1.24 | 10.73 |
| | (SEQ TD NO: 84) | | | | | | | | | | | | | | | |
| AVT915 | XNSRGDYXSL | 51.40 | 42.13 | 100.58 | 33.76 | 36.91 | 55.97 | 29.36 | 45.67 | 10.32 | 21.99 | 2.10 | 1.56 | 1.31 | 2.31 | 1.52 |
| | (SEQ ID NO: 67) | | | | | | | | | | | | | | | |
| AV9ML014 | NNNRGDYNSL | 17.80 | 16.24 | 41.51 | 13.83 | 11.62 | 20.16 | 14.20 | 16.49 | 7.08 | 13.44 | 1.23 | 0.73 | 0.95 | 0.86 | 0.56 |
| | (SEQ ID NO: 85) | | | | | | | | | | | | | | | |
| AVT906 | SAQLHKGIDI | 3.19 | 3.38 | 5.55 | 1.99 | 2.56 | 2.96 | 3.67 | 2.40 | 8.35 | 7.82 | 1.17 | 0.93 | 0.96 | 1.90 | 0.95 |
| | (SEQ ID NO: 171) | | | | | | | | | | | | | | | |
| AV9ML016 | SAQRGDKLST | 4.20 | 2.51 | 5.87 | 0.22 | 2.38 | 6.46 | 4.84 | 3.91 | 7.97 | 13.38 | 0.81 | 0.59 | 0.49 | 1.54 | 1.28 |
| | (SEQ ID NO: 176) | | | | | | | | | | | | | | | |
| AVT903 | SAQRGDTNNTNM | 1.41 | 1.00 | 1.46 | 0.75 | 0.63 | 0.90 | 1.47 | 1.03 | 2.54 | 12.69 | 0.15 | 0.14 | 0.24 | 0.05 | 0.19 |
| | (SEQ ID NO: 169) | | | | | | | | | | | | | | | |
| AV9ML018 | SAQLNTNNSFTK | 0.29 | 0.02 | 0.04 | 10.00 | 0.09 | 0.00 | 0.02 | 0.06 | 0.13 | 0.10 | 0.04 | 0.04 | 0.04 | 0.10 | 0.04 |
| | (SEQ ID NO: 177) | | | | | | | | | | | | | | | |
| AVT905 | SAQSRGDRPM | 0.18 | 0.03 | 0.24 | 2.82 | 0.06 | 0.04 | 0.03 | 0.00 | 0.12 | 0.20 | 0.40 | 0.31 | 0.18 | 2.58 | 0.38 |
| | (SEQ ID NO: 170) | | | | | | | | | | | | | | | |
| MyoAAV 1A | SAQRGDLTTP | 6.60 | 6.01 | 6.53 | 6.11 | 2.85 | 8.86 | 6.00 | 10.04 | 14.11 | 4.89 | 1.10 | 1.17 | 1.10 | 2.41 | 0.37 |
| | (SEQ ID NO: 109) | | | | | | | | | | | | | | | |
| MyoAAV 2A | GPGRGDQTTL | 3.43 | 2.37 | 2.88 | 1.61 | 1.17 | 5.18 | 1.26 | 3.70 | 2.03 | 3.06 | 0.41 | 0.27 | 0.22 | 0.66 | 0.36 |
| | (SEQ ID NO: 110) | | | | | | | | | | | | | | | |
| MyoAAV 3A | SAQRGDYVGL | 16.76 | 16.72 | 33.01 | 12.55 | 12.45 | 20.64 | 9.88 | 18.65 | 14.69 | 8.62 | 0.69 | 0.78 | 0.56 | 2.13 | 0.34 |
| | (SEQ ID NO: 113) | | | | | | | | | | | | | | | |
| MyoAAV 4A | SNSRGDYNSL | 34.94 | 30.00 | 68.40 | 17.62 | 25.79 | 36.65 | 20.81 | 29.21 | 8.21 | 16.66 | 1.03 | 0.98 | 0.76 | 3.30 | 10.52 |
| | (SEQ ID NO: 114) | | | | | | | | | | | | | | | |
| AAV9 | SAQ | 1.00 | 1.00 | 1.00 | 1.00 | 1.00 | 1.00 | 1.00 | 1.00 | 1.00 | 1.00 | 1.00 | 1.00 | 1.00 | 1.00 | 1.00 |

All the capsids (except AVT905 and AV9ML018) showed improvements in the muscle compared to AAV9. And AVT919, AVT913, AVT915, AVT914, AVT917, AVT916, AV9ML012 showed improvements at mRNA level in the various muscle tissues compared to MyoAAV 4A (Table 5).

TABLE 5
Ranking of muscle-tropic capsids in NHP
(mRNA level).

| | | Mean fold |
|---|---|---|
| | 586-588 of AAV9 | change |
| Variant name | VP1 reconstituted | in muscles |
| AVT919 | MKVRGDTNKL | 55.17 |
| | (SEQ ID NO: 75) | |
| AVT913 | ENRRGDTNAL | 45.21 |
| | (SEQ ID NO: 63) | |
| AVI915 | NNSRGDYNSL | 42.81 |
| | (SEQ ID NO: 67) | |
| AVT914 | NNSRGDHNSL | 41.86 |
| | (SEQ ID NO: 65) | |
| AVT917 | NNARGDHNSL | 36.00 |
| | (SEQ ID NO: 71) | |
| AVT916 | INNLRGDHINSL | 33.97 |
| | (SEQ ID NO: 69) | |
| AV9ML012 | INSRGDHNVL | 32.04 |
| | (SEQ ID NO: 84) | |
| MyoAAV 4A | SNSRGDYNSL | 28.83 |
| | (SEQ ID NO: 114) | |
| AV9ML004 | NNERGDRNSL | 24.45 |
| | (SEQ ID NO: 81) | |
| AVT918 | GNIRGDHNTL | 22.42 |
| | (SEQ ID NO: 73) | |
| AV9ML005 | NNSRGDHNIL | 22.40 |
| | (SEQ ID NO: 82) | |
| AV9ML007 | ENKRGDHNSL | 19.05 |
| | (SEQ ID NO: 83) | |
| AV9ML014 | NNKRGDYNSL | 17.24 |
| | (SEQ ID NO: 85) | |
| MyoAAV 3A | SAQRGDYVGL. | 15.30 |
| | (SEQ ID NO: 113) | |
| AV9ML002 | NNNRGDHNTL | 10.92 |
| | (SEQ ID NO: 80) | |
| AV9ML001 | NNNRGDNNYL | 9.26 |
| | (SEQ ID NO: 79) | |
| MyoAAV 1A | SAQRADLTTP | 6.20 |
| | (SEQ ID NO: 109) | |
| AV9ML016 | SAQRGDKLSI | 5.18 |
| | (SEQ ID NO: 176) | |
| AVT906 | SAQLIIKGIDI | 4.19 |
| | (SEQ TD NO: 171) | |
| MyoAAV 2A | GPGRGDOTTI | 2.67 |
| | (SEQ ID NO: 110) | |
| AVT903 | SAQRGDINNINM | 1.39 |
| | (SEQ ID NO: 169) | |
| AAV9 | SAQ | 1.00 |
| AVT905 | SAQSRGDRPM | 0.37 |
| | (SEQ ID NO: 170) | |
| AV9ML018 | SAQLNTNNSFTK | 0.08 |
| | (SEQ ID NO: 177) | |

AVT918, AV9ML005, AVT913, AVT919, AV9ML002 and AVT916 showed improvement in the brain of NHP after intravenous administration; their mRNA levels were more than 2-fold the mRNA level of AAV9 (Table 6)

TABLE 6
Ranking of brain-tropic capsids in NHP
(mRNA level).

| | 586-588 of AAV9 | |
|---|---|---|
| Variant name | VP1 reconstituted | Mean FC in brain |
| AVT918 | GNIRGDENTL | 5.97 |
| | (SEQ ID NO: 73) | |
| AV9ML005 | NNSRGDINTL | 5.96 |
| | (SEQ ID NO: 82) | |
| AVT913 | ENRRGDINAL | 3.81 |
| | (SLQ ID NO: 63) | |
| AVT919 | MNVRGDINKL | 3.41 |
| | (SEQ ID NO: 75) | |
| AV9ML002 | NNNRGDENTL | 2.65 |
| | (SEQ ID NO: 80) | |
| AVT916 | NNLRGDHNSL | 2.02 |
| | (SEQ ID NO: 69) | |
| AVT915 | NNSRGDYNSL | 1.92 |
| | (SEQ ID NO: 67) | |
| MyoAAV 4A | SNSRGDYNSL | 1.91 |
| | (SEQ ID NO: 114) | |
| AVT917 | NNARGDHNSL | 1.91 |
| | (SEQ ID NO: 71) | |
| AV9ML004 | NNERGDRNSL | 1.82 |
| | (SEQ ID NO: 81) | |
| AVT914 | NNSRGDIINSL | 1.70 |
| | (SEQ ID NO: 65) | |
| AVT905 | SAQSRGDRPM | 1.48 |
| | (SEQ ID NO: 170) | |
| AVT906 | SAQLHKGIDI | 1.42 |
| | (SEQ ID NO: 171) | |
| AV9ML016 | SAQRGDKLSI | 1.41 |
| | (SEQ ID NO: 176) | |
| MyoAAV 1A | SAQRGDLTTP | 1.39 |
| | (SEQ ID NO: 109) | |
| AV9ML007 | ENKRGDHNSL | 1.25 |
| | (SEQ ID NO: 83) | |
| MyoAAV 3A | SAQRGDYVGL | 1.23 |
| | (SEQ ID NO: 113) | |
| AAV9 | SAQ | 1.00 |
| AV9ML012 | INSRGDHNVL | 0.99 |
| | (SEQ ID NO: 84) | |
| AV9ML014 | NNNRGDYNSL | 0.71 |
| | (SEQ ID NO: 85) | |
| AV9ML001 | NNNRGDNNYL | 0.64 |
| | (SEQ ID NO: 79) | |
| MyoAAV 2A | GPORGDQTTL | 0.51 |
| | (SEQ ID NO: 110) | |
| AVT903 | SAQRGDTNKTNM | 0.12 |
| | (SEQ ID NO: 169) | |
| AV9ML018 | SAQLNTNNSFTK | 0.07 |
| | (SEQ ID NO: 177) | |

AVT914, AVT916, AVT917, AVT918, AVT913 and AVT915 showed improvement in the liver of NHP after intravenous administration; their mRNA levels were more than 2-fold the mRNA level of AAV9 (Table 7).

TABLE 7
Ranking of liver-tropic capsids in NHP
(mRNA level).

| | 586-588 of AAV9 | |
|---|---|---|
| Variant name | VPI reconstituted | Mean FC in liver |
| AVT914 | NNSRGDENSL | 2.67 |
| | (SEQ ID NO: 65) | |
| AVT916 | NNLRGDHNSL | 2.56 |
| | (SEQ ID NO: 69) | |
| AVT917 | NNARGDENSL | 2.46 |
| | (SEQ ID NO: 71) | |
| AVT918 | GNIRGDENTL | 2.42 |
| | (SEQ ID NO: 73) | |
| AVT913 | ENRRGDTNAL | 2.28 |
| | (SEQ ID NO: 63) | |
| AVT915 | NNSRGDYNSL | 2.10 |
| | (SEQ ID NO: 67) | |
| AV9ML004 | NNERGDRNSL | 1.69 |
| | (SEQ ID NO: 81) | |
| AV9ML005 | NNSRGDENTL | 1.52 |
| | (SEQ ID NO: 82) | |
| AV9ML002 | NNNRGDHNTL | 1.49 |
| | (SEQ ID NO: 80) | |
| AVT919 | MNVRGDINKL | 1.45 |
| | (SEQ ID NO: 75) | |
| AV9ML014 | KNNRGDYNSL | 1.23 |
| | (SEQ ID NO: 85) | |
| AVT906 | SAQLHKGIDI | 1.17 |
| | (SEQ ID NO: 171) | |
| MyoAAY 1A | SAQRGDLTIP | 1.10 |
| | (SEQ ID NO: 109) | |
| MyoAAV 4A | SKSRGDYKSL | 1.03 |
| | (SEQ ID NO: 114) | |
| AV9ML007 | ENKRGDHNSL | 1.02 |
| | (SEQ ID NO: 83) | |
| AAV9 | SAQ | 1.00 |
| AV9ML016 | SAQRGDKLSI | 0.81 |
| | (SEQ ID NO: 176) | |
| AV9ML001 | NNNRGDNNYL | 0.78 |
| | (SEQ ID NO: 79) | |
| MyoAAV 3A | SAQRGDYVGL | 0.69 |
| | (SEQ ID NO: 113) | |
| AV9ML012 | INSRGDIINVL | 0.47 |
| | (SEQ ID NO: 84) | |
| MyoAAV 2A | GPGRGDQTTE | 0.41 |
| | (SEQ ID NO: 110) | |
| AVT905 | SAQSRGDRPM | 0.40 |
| | (SEQ ID NO: 170) | |
| AVT903 | SAQRGDTNNTNM | 0.15 |
| | (SEQ ID NO: 169) | |
| AV9ML018 | SAQLNTNNSFTK | 0.04 |
| | (SEQ ID NO: 177) | |

In addition, 27 capsid mutants (AV9ML025-051) were identified to be superior at mRNA level in NHP, compared to benchmark MyoAAV 4A (Table 8).

TABLE 8
Additional 27 capsid mutants generated during the NHP validation study (Fold change over MyoAAV 4A).

| | 586-588 of | | | | | | | | | | | | middle | middle | | |
|---|---|---|---|---|---|---|---|---|---|---|---|---|---|---|---|---|
| Capsid | AAV9 VPI | tibialis | | | quadri- | gastric- | rectus | | pectoralis | | | | front | rear | front | rear |
| name | reconstituted | anterior | biceps | triceps | ceps | nemius | abdominis | diaphragm | major | atrium | ventricle | liver | brain | brain | brain | brain |
| MyoAAV 4A | SKSRGDYNSL | 1.00 | 1.00 | 1.00 | 1.00 | 1.00 | 1.00 | 1:00 | 1.00 | 1.00 | 1.00 | 1.00 | 1.00 | 1.00 | 1.00 | 1.00 |
| | (SEQ ID NO: 114) | | | | | | | | | | | | | | | |
| AAV9 | SAQ | 0.03 | 0.04 | 0.02 | 0.06 | 0.04 | 0.03 | 0.05 | 10.04 | 10.12 | 0.12 | 0.97 | 1.05 | 1.36 | 0.34 | 1.95 |
| AV9ML025 | SNERGDHNTL | 3.08 | 4.63 | 3.88 | 0.85 | 3.49 | 4.51 | 4.19 | 4.67 | 1.91 | 1.95 | 0.72 | 0.39 | 0.38 | 0.19 | 0.22 |
| | (SEQ ID NO: 116) | | | | | | | | | | | | | | | |
| AV9ML026 | SNSRGDHNSL | 9.53 | 14.66 | 10.10 | 9.68 | 10.27 | 14.69 | 13.17 | 14.04 | 8.33 | 9.87 | 1.26 | 0.40 | 0.70 | 10.11 | 0.24 |
| | (SEQ ID NO: 118) | | | | | | | | | | | | | | | |
| AV9ML027 | ENRRGDHNSL | 6.75 | 7.13 | 4.42 | 26.21 | 6.36 | 13.29 | 9.03 | 19:18 | 6.87 | 8.18 | 2:45: | 2.00 | 1.37 | 0.55 | 0.97 |
| | (SIQ ID NO: 120) | | | | | | | | | | | | | | | |
| AV9ML028 | GXSRGDYNSE | 5.20 | 8.41 | 4.61 | 10.62 | 7.52 | 10.34 | 8.05 | 11.27 | 3.23 | 3.05 | 1.86 | 3.22 | 1.71 | 0.31 | 0.59 |
| | (SEQ ID NO: 122) | | | | | | | | | | | | | | | |
| AV9ML029 | KYSRGDRNSL | 4.49 | 3.08 | 2.85 | 0.48 | 2.14 | 3.94 | 3.92 | 5.25 | 4.40 | 4.44 | 2.74 | 1.14 | 1.39 | 0.34 | 0.68 |
| | (SEQ ID NO: 124) | | | | | | | | | | | | | | | |
| AV9ML030 | MNVRGDTNSL | 3.02 | 2.86 | 2.24 | 47.23 | 3.61 | 3.60 | 4.39 | 7.74 | 2.57 | 3.83 | 3.24 | 0.88 | 1.27 | 25.33 | 1.34 |
| | (SEQ ID NO: 126) | | | | | | | | | | | | | | | |
| AV9ML031 | SKARGDHNSL | 18.91 | 14.23 | 10.92 | 9.87 | 12.14 | 11.68 | 7.54 | 21.42 | 5.23 | 4.97 | 2.34 | 0.64 | 1.06 | 0.11 | 0.54 |
| | (SEQ ID NO: 128) | | | | | | | | | | | | | | | |
| AV9ML032 | GNSRGDHNTL | 9.78 | 13.72 | 7.70 | 0.20 | 3.80 | 18.72 | 8.43 | 16.59 | 4.71 | 5.69 | 6.25 | 1.49 | 0.76 | 0.60 | 1.32 |
| | (SEQ ID NO: 130) | | | | | | | | | | | | | | | |
| AV9ML033 | NNNRGDHNAL | 11.84 | 4.76 | 2.46 | 0.53 | 5.89 | 6.93 | 7.26 | 12.37 | 7.02 | 5.50 | 4.09 | 1.38 | 1.44 | 2.09 | 1.71 |
| | (SEQ ID NO: 132) | | | | | | | | | | | | | | | |
| AV9ML034 | NNLRGDRNSL | 0.13 | 7.80 | 6.30 | 0.45 | 6.93 | 12.80 | 10.73 | 19.65 | 9.58 | 6.96 | 3.33 | 0.57 | 0.93 | 0.25 | 0.55 |
| | (SEQ ID NO: 134) | | | | | | | | | | | | | | | |
| AV9ML035 | DARRGDTNAL | 1.55 | 2.53 | 2.56 | 3.08 | 2.22 | 4.25 | 1.69 | 1.94 | 2.64 | 4.17 | 3.33 | 6.14 | 12.00 | 2.37 | 3.46 |
| | (SIQ ID NO: 136) | | | | | | | | | | | | | | | |
| AV9ML036 | ENERGDHNSL | 25.69 | 6.55 | 7.28 | 0.95 | 7.13 | 10.38 | 9.36 | 4.86 | 8.20 | 6.70 | 3.32 | 1.18 | 11.71 | 0.79 | 1.63 |
| | (SEQ ID NO: 138) | | | | | | | | | | | | | | | |
| AV9ML037 | INNARGDTNAL | 2.57 | 4.03 | 5.45 | 3.00 | 3.05 | 8.93 | 3.83 | 16.17 | 5.65 | 8.60 | 4.12 | 30.58 | 5.19 | 0.68 | 4.05 |
| | (SHQ ID NO: 140) | | | | | | | | | | | | | | | |
| AV9ML038 | SKLRGDHNSL | 0.25 | 3.29 | 11.47 | 117.84 | 6.94 | 4.62 | 4.97 | 22.51 | 1.84 | 1.39 | 2.53 | 0.62 | 1.19 | 0.23 | 0.77 |
| | (SEQ ID NO: 142) | | | | | | | | | | | | | | | |
| AV9ML039 | MSVRGDTNKL | 1.80 | 1.79 | 1.65 | 1.04 | 1.37 | 3.41 | 3:95 | 2.99 | 1.32 | 1.78 | 1.43 | 1.21 | 1.58 | 1.62 | 2.56 |
| | (SEQ ID NO: 144) | | | | | | | | | | | | | | | |
| AV9ML040 | MNVRGDTNKP | 3.23 | 2.79 | 1.91 | 1.10 | 2.55 | 4.14 | 4.81 | 3.32 | 1.59 | 2.08 | 2.50 | 1.32 | 1.87 | 2.03 | 2.89 |
| | (SEQ TD NO: 146) | | | | | | | | | | | | | | | |
| AV9ML041 | NNLRGDTNAL | 3.78 | 1.74 | 0.35 | 1.90 | 2.57 | 5.33 | 2.72 | 12.56 | 1.80 | 1.24 | 2:90 | 1.28 | 2.35 | 0.57 | 2.48 |
| | (SEQ ID NO: 148) | | | | | | | | | | | | | | | |
| AV9ML042 | INSRGDYKVL | 9.43 | 2.17 | 0.70 | 10.73 | 0.88 | 1.97 | 2.18 | 2.14 | 1.16 | 1.51 | 4.02 | 0.28 | 0.18 | 0.27 | 0.94 |
| | (SEQ ID NO: 150) | | | | | | | | | | | | | | | |
| AV9ML043 | KARRGDTNAL | 5.32 | 1.55 | 0.55 | 135.06 | 1.03 | 1.53 | 1.41 | 1.57 | 2.69 | 3.91 | 6.51 | 1.23 | 1.53 | 2.37 | 1.06 |
| | (SEQ ID NO: 152) | | | | | | | | | | | | | | | |
| AV9ML044 | ENERGDRNSL | 14.88 | 60.60 | 36:03 | 23.52 | 50.84 | 91.83 | 57.59 | 97.70 | 12.33 | 19.59 | 3.58 | 1.72 | 1.12 | 1.53 | 0.26 |
| | (SEQ ID NO: 154) | | | | | | | | | | | | | | | |
| AV9ML045 | MNARGDTNKL | 91.60 | 102.44 | 74.76 | 99.72 | 48.00 | 179.45 | 156.52 | 208.38 | 29.76 | 54.78 | 2.57 | 1.27 | 1.73 | 1.70 | 3.06 |
| | (SEQ ID NO: 156) | | | | | | | | | | | | | | | |
| AV9ML046 | GKIRGDRNTL | 17.34 | 67.90 | 23.06 | 0.72 | 31.13 | 68.20 | 54.70 | 121.98 | 25.24 | 28.63 | 11.31 | 2.00 | 2.12 | 3.30 | 5.37 |
| | (SEQ ID NO: 158) | | | | | | | | | | | | | | | |
| AV9ML047 | GNIRGDHNAL | 110.70 | 41.49 | 20.28 | 0.90 | 26.48 | 75.85 | 83.17 | 126.33 | 27.91 | 35.97 | 6.37 | 1.66 | 1.85 | 3.99 | 3.89 |
| | (SEQ ID NO: 160) | | | | | | | | | | | | | | | |
| AV9ML048 | NNRRGDHNSL | 0.98 | 2.10 | 1.15 | 1.61 | 1.49 | 2.71 | 1.87 | 3.88 | 1.39 | 1.11 | 3.25 | 11.37 | 1.60 | 0.57 | 2.15 |
| | (SEQ ID NO: 162) | | | | | | | | | | | | | | | |
| AV9ML049 | TNIRGDYNSL | 0.95 | 1.35 | 1.65 | 1.64 | 1.22 | 1.33 | 1.20 | 1.16 | 1.51 | 1.50 | 2.96 | 2.32 | 2.27 | 1.60 | 3.15 |
| | (SEQ ID NO: 164) | | | | | | | | | | | | | | | |
| AV9ML050 | RNLRGDHNTL | 1.40 | 1.90 | 0.94 | 1.25 | 0.98 | 2.46 | 3.15 | 3.97 | 1.93 | 1.88 | 3.98 | 2.48 | 3.49 | 6.18 | 7.87 |
| | (SEQ ID NO: 166) | | | | | | | | | | | | | | | |
| AV9ML051 | CNSRGDHNTL | 1.16 | 1.97 | 1.31 | 0.73 | 1.44 | 1.75 | 2.07: | 2:25 | 1.27 | 1.19 | 1.64 | 0.83 | 1.22 | 1.98 | 3.08 |
| | (SEQ ID NO: 168) | | | | | | | | | | | | | | | |

### Example 8: Engineered AAV9 Variants for Efficient Muscle Transduction

Systematic administration of Adeno-associated virus (AAV) vectors expressing functional genes represents a promising treatment for muscular dystrophy. However, very high dose of AAV is required due to its low transduction efficiency in the muscle after intravenous delivery. This study aimed to engineer novel AAV capsids with enhanced transduction efficiency in muscles using an AI-aided AAV capsid evolution discovery technology. Based on AAV9 capsid, random peptide insertion AAV capsid libraries were generated and screened in both mice and non-human primates (NHP). The screening process relied on muscle-specific viral mRNA recovery by using an in-house developed potent synthetic muscle promoter SCC45. After two rounds of screening in 3 mouse strains (C57BL/6, BALB/c and DBA/2j-mdx) and one round in NHP, the top capsid candidates were selected for individual validation. By using GFP and firefly luciferase reporter genes, several variants showed dramatic improvements (up to 113 folds) in the mouse skeletal muscles compared to AAV9. One variant AVT913 showed 26-fold enhanced transduction at mRNA level in the heart.

Of note, variant AVT919 showed significant improvements in triceps and tibialis anterior over MyoAAV2A, a benchmark myotropic AAV capsid. Although the majority of top variants showed improved muscle transduction and reduced liver targeting, one particular variant AVT908 demonstrated enhanced efficiency in both muscle and liver.

To confirm if similar tissue-tropic patterns could be recapitulated in NHP, those variants together with additional candidates were individually packaged and intravenously injected to adult cynomolgus macaques. The characteristic results revealed the cross-species translatability. Those engineered myotropic AAV capsids would provide efficient delivery platforms for muscle-targeting gene therapy.

### INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A variant adeno-associated virus serotype 9 (AAV9) capsid protein comprising the amino acid sequence of SEQ ID NO:75, SEQ ID NO:71, SEQ ID NO:63, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:65, SEQ ID NO:67, SEQ ID NO:69, SEQ ID NO:73, SEQ ID NO:77, SEQ ID NO:79, SEQ ID NO:80, SEQ ID NO:81, SEQ ID NO:82 SEQ ID NO:83, SEQ ID NO:84, SEQ ID NO:85, SEQ ID NO:86, SEQ ID NO:87, SEQ ID NO:116, SEQ ID NO:118, SEQ ID NO:120, SEQ ID NO:122, SEQ ID NO:124, SEQ ID NO: 126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO: 150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, or SEQ ID NO:168.

2. The variant AAV9 capsid protein of claim 1, which comprises the amino acid sequence of SEQ ID NO:6-33, SEQ ID NO:115, SEQ ID NO:117, SEQ ID NO: 119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, or SEQ ID NO:167.

3. The variant AAV9 capsid protein of claim 2, wherein the variant AAV9 capsid protein comprising SEQ ID NO: 43, SEQ ID NO: 9, SEQ ID NO: 51, SEQ ID NO: 12, SEQ ID NO: 63, SEQ ID NO: 17, SEQ ID NO: 65, SEQ ID NO: 18, SEQ ID NO: 67, SEQ ID NO: 19, SEQ ID NO: 69, SEQ ID NO: 20, SEQ ID NO: 71, SEQ ID NO: 21, SEQ ID NO: 73 or SEQ ID NO: 22 is associated with an increased tropism for liver relative to a wild-type AAV9 capsid protein comprising the amino acid sequence of SEQ ID NO: 2.

4. The variant AAV9 capsid protein of claim 2, SEQ ID NO: 63, SEQ ID NO: 17, SEQ ID NO: 69, SEQ ID NO: 20, SEQ ID NO: 73, SEQ ID NO: 22, SEQ ID NO: 75, SEQ ID NO: 23, SEQ ID NO: 80, or SEQ ID NO: 26 is associated with an increased tropism for brain relative to a wild-type AAV9 capsid protein comprising the amino acid sequence of SEQ ID NO: 2.

5. The variant AAV9 capsid protein of claim 4, which is associated with an increased tropism for skeletal muscle and/or heart muscle relative to the wild-type AAV9 capsid protein.

6. The variant AAV9 capsid protein of claim 4, which is associated with an increased tropism for biceps, triceps, quadriceps, tibialis anterior, gastrocnemius, rectus abdominis, diaphragm, pectoralis major, heart atrium muscle, and/or heart ventricle muscle relative to the wild-type AAV9 capsid protein.

7. A recombinant AAV9 particle comprising the variant AAV9 capsid protein of claim 1.

8. A pharmaceutical composition comprising the recombinant AAV9 particle of claim 7 and a pharmaceutically acceptable carrier.

9. A polynucleotide encoding the variant AAV9 capsid protein of claim 1.

10. A vector comprising the polynucleotide of claim 9.

11. A host cell comprising the polynucleotide of claim 9.

12. A population of host cells stably transduced by the recombinant AAV9 particle of claim 7.

13. A pharmaceutical composition comprising the population of host cells of claim 12 and a pharmaceutically acceptable carrier.

14. A method of delivering a biologic molecule to one or more ex vivo or in vitro target cells, comprising transducing the one or more target cells with the recombinant AAV9 particle of claim 7.

15. A method of delivering a biologic molecule to one or more in vivo target cells in a subject, comprising administering to the subject the recombinant AAV9 particle of claim 7.

16. The method of claim 14, wherein the one or more target cells are one or more muscle cells, one or more liver cells, and/or one or more brain cells.

17. A method of treating a disease or disorder in a subject in need thereof, comprising administering to the subject the recombinant AAV9 particle of claim 7.

18. The method of claim 15, wherein the subject is a human.

19. A method of producing a recombinant AAV9 particle, comprising culturing the host cell of claim 11.

20. The method of claim 15, wherein the one or more target cells are one or more muscle cells, one or more liver cells, and/or one or more brain cells.
